(19) United States
(12) Patent Application Publication
(10) Pub. No.: US 2026/0273075 A1
(43) Pub. Date: Sep. 17, 2026

(54) CARBOHYDRATE-POLYMER CONJUGATES FOR TUNABLE PROTEIN BINDING AND METHODS OF USE

(71) Applicant: Board of Regents, The University of Texas System, Austin, TX (US)

(72) Inventors: Cassandra Callmann, Austin, TX (US); Cole Williams, Austin, TX (US); Zamira Harris-Ryden, Austin, TX (US); Muhammad Waqas Ishaq, Austin, TX (US); Daniel Stone, Austin, TX (US)

(21) Appl. No.: 19/469,819

(22) PCT Filed: Feb. 16, 2024
(86) PCT No.: PCT/US2024/016211

§ 371 Date: Sep. 26, 2025

Related U.S. Application Data
(60) Provisional application No. 63/492,682, filed on Mar. 28, 2023.

(51) Int. Cl. A61K 47/58; A61K 31/7004; A61P 35/00

(52) U.S. Cl. CPC A61K 47/58; A61K 31/7004; A61P 35/00

(57) ABSTRACT

The present invention relates to a carbohydrate-polymer conjugate comprising a repeat unit, a divalent linker, and a carbohydrate and compositions thereof, wherein the repeat unit comprises, in part, a linker and a carbohydrate, as well as a method of synthesizing a carbohydrate-polymer conjugate using ring-opening metathesis polymerization (ROMP) and a method comprising the step of administering a composition comprising the carbohydrate-polymer conjugate, wherein the composition can bind to a lectin.

### CROSS-REFERENCE TO RELATED APPLICATIONS

This application claims priority to International Application No. PCT/US2024/016211, filed Feb. 16, 2024, which claims priority to U.S. Provisional Patent Application No. 63/492,682, filed Mar. 28, 2023, the contents of each of which are incorporated by reference herein in their entireties.

### BACKGROUND OF THE INVENTION

Nature has evolved carbohydrates as effectors of key biological processes, including molecular recognition, intercellular communication, cellular adhesion, and signal transduction (Godula, 2012, J. Am. Chem. Soc., 134, 15732; Cobb, 2005, Eur. J. Immunol., 35, 352). Given its importance, there is broad interest in the fields of chemistry, materials science, and chemical biology in understanding the molecular basis for this complex communication (Hendrikse et al., 2019, J. Am. Chem. Soc., 141, 13877; Bertozzi, 2001, Science, 291, 2357). Moreover, synthetic glycomaterials that can probe and manipulate biological processes hold significant importance in both biotechnological (Richards, 2021, JACS Au, 1, 2089; Lutz et al., 2022, Materials Today Bio, 13, 100203; Singh et al., 2023, J. Am. Chem. Soc., 145, 1668; Isono et al., 2020, Macromolecules, 53, 5408; Echeverri, 2022, Molecules, 27, 8533; Mancini et al., 2012, J. Am. Chem. Soc., 134, 8474) and biomedical (Stenzel, 2022, Macromolecules, 55, 4867; Clauss et al., 2021, Nat. Comm., 12, 6472; Gim et al., 2019, Wires Nanomed. Nanobiol., 11, e1558; Zhao et al., 2022, Biomacromolecules, 23, 543; Mastrotto et al., 2022, J. Am. Chem. Soc., 144, 23134; O'Leary et al., 2022, Nat. Chem. Biol., 18, 634; Restruccia et al., 2016, J. Mat. Chem. B, 4, 1568; Thodikayil et al., 2021, ACS Appl. Bio. Mat., 4, 2907) contexts.

Strikingly, the entire mammalian glycome is composed of only ~10 different monosaccharides (Purcell, 2019, Interface Focus, 9, 20180080). As such, the complex, multifunctional role of carbohydrates relies not only on their chemical composition, but also on their macromolecular presentation on scaffold proteins and lipids, coupled with their supramolecular assembly and structural ensemble with other glycoconjugates (Dam, 2010, Glycobiology, 20, 270; Miura et al., 2016, Chem. Rev., 116, 1673). In the body, carbohydrates are primarily presented as dense sidechains of oligosaccharides on polypeptide backbones, forming natural “bottlebrush” polymers on proteins and cell surfaces (Miura et al., 2016, Chem. Rev., 116, 1673; Ng et al., 2003, J. Struct. Biol., 143, 242). This multimeric display leads to high avidity with their protein (lectin) binding partners (K_{d}~10-6), despite weak individual carbohydrate-protein binding affinities (K_{d}~10-3)—a well-documented phenomenon termed the “glycoside cluster effect”. In this way, Nature leverages the power of polymers to generate highly selective and specific molecular recognition motifs that are physiologically dynamic.

A fundamental understanding of the macromolecular basis of carbohydrate recognition is essential for deepening understanding of the natural world (Hartweg et al., 2021, JACS Au, 1, 1621; Kiessling, 2013, Chem. Soc. Rev., 42, 4476; Huang, 2016, Glycobiology, 26, 797), as well as to enable the development of new probes and therapeutics. Over the past ~40 years, a significant body of research has been built up on understanding how carbohydrate multivalency influences function (Gordon et al., 1998, Bioorg. Med. Chem., 6, 1293; Lees et al., 1998, J. Med. Chem., 37, 3419; Abdouni et al., 2020, Biomacromolecules, 21, 3756). However, many analyses focus on plant lectins (Kanai et al., 1997, J. Am. Chem. Soc., 119, 9931; Cairo et al., 2002, J. Am. Chem. Soc., 124, 1615; Mortell et al., 1996, J. Am. Chem. Soc., 118, 2297), which, although significant, lack relevance to human health. Moreover, the majority of glycopolymers comprise statistical copolymers, utilize graft-to techniques (Manning et al., 1997, J. Am. Chem. Soc., 119, 3161; Strong, 1999, J. Am. Chem. Soc., 121, 6193; Kamitakahara et al., 1998, Angew. Chem. Int. Ed., 37, 1524), and/or employ protecting groups that need to be removed post-polymerization (Manning et al., 1997, J. Am. Chem. Soc., 119, 3161; Strong, 1999, J. Am. Chem. Soc., 121, 6193; Kamitakahara et al., 1998, Angew. Chem. Int. Ed., 37, 1524; Sheng et al., 2013, J. Am. Chem. Soc., 135, 10898; Oh et al., 2013, Angew. Chem. Int. Ed., 52, 11796). This results in materials with stochastic local compositions, which restricts batch-to-batch reproducibility and complicates systematic analyses. Indeed, recent developments in discrete glycomacromolecules with absolute sequence control underscore the necessity for well-defined glycomaterials to analyze these complex interactions in a systematic manner.

Thus, there is a need in the art for compositions and methods for making and use of well-defined glycomaterials and carbohydrate-polymer conjugates. The present invention satisfies this unmet need.

### SUMMARY OF THE INVENTION

The present invention is drawn to, in part, a carbohydrate-polymer conjugate comprising a structure represented by Formula (I):

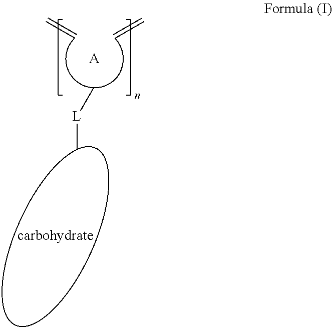

wherein: A represents a repeat unit selected from the group consisting of C₅-C₇ alkyl, C₅-C₇ heteroalkyl, C₅-C₇ aryl, C₅-C₇ heteroaryl, or C₅-C₇ cycloalkyl; n represents an integer from 10-500; L represents a divalent organic linker; and wherein L forms a direct bond to an anomeric position of the carbohydrate.

In one embodiment, the carbohydrate-polymer conjugate of claim 1, wherein ring A is represented by one of the following structures:

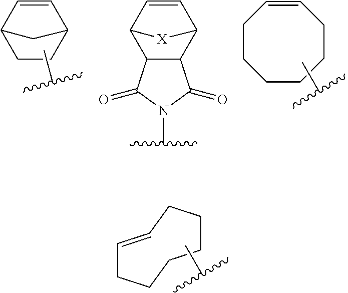

wherein the wave line represents a bond to L; X represents O, S, NRI, or CR²R³; R¹, R² and R³ each independently represent a substituent selected from the group consisting of hydrogen, halide, hydroxy, C₁-C₃₀ alkyl, C₁-C₃₀ alkyl halide, C₁-C₃₀ alkoxy, and any combination thereof, and each structure is independently optionally further substituted. In one embodiment, the polymer backbone is unsaturated.

In one embodiment, L is represented by one of the following structures:

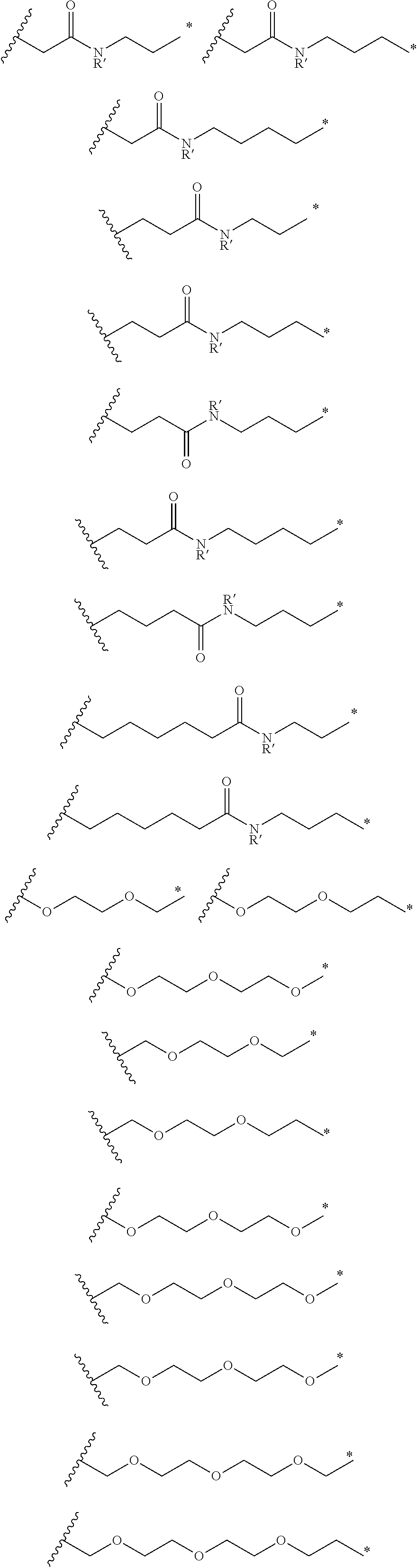

wherein the wavy line represents a bond to ring A; * represents a bond to the carbohydrate; and R′ represents a substituent selected from the group consisting of hydrogen, halide, hydroxy, C₁-C₃₀ alkyl, C₁-C₃₀ alkyl halide, C₁-C₃₀ alkoxy, and any combination thereof. In one embodiment, L comprises a C₁-C₃₀ alkyl which is optionally further substituted.

In one embodiment, the carbohydrate is selected from the group consisting of a monosaccharide, a disaccharide, an oligosaccharide, a polysaccharide, any stereochemical isomer thereof, and any combination thereof. In one embodiment, the carbohydrate is selected from the group consisting of galactose, glucose, mannose, allose, altrose, gulose, idose, talose, psicose, fructose, sorbose, tagatose, and any stereochemical isomer thereof. In one embodiment, the carbohydrate-polymer conjugate is a copolymer comprising a repeat unit of at least two monomers, wherein at least one monomer comprises galactose. In one embodiment, the carbohydrate is acetylated.

The present invention is further drawn to, in part, a composition comprising the carbohydrate-polymer conjugate, and a method of synthesizing a carbohydrate-polymer conjugate using ring-opening metathesis polymerization (ROMP), comprising the steps of: providing a monomer; polymerizing the monomer using a ROMP catalyst; and isolating the carbohydrate-polymer conjugate; wherein the monomer is represented by Formula (I):

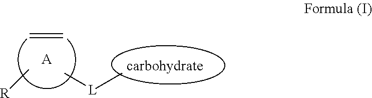

wherein: R represents a substituent on ring A selected from the group consisting of hydrogen, halide, hydroxy, C₁-C₃₀ alkyl, C₁-C₃₀ alkyl halide, C₁-C₃₀ alkoxy, and any combination thereof, and L represents a divalent organic linker.

In one embodiment, the degree of polymerization is about 10 to about 500. In one embodiment, the polymerization is performed at about 60° C.

The present invention is further drawn to, in part, a method comprising the step of administering to the subject a composition comprising a carbohydrate-polymer conjugate comprising a structure represented by Formula (I):

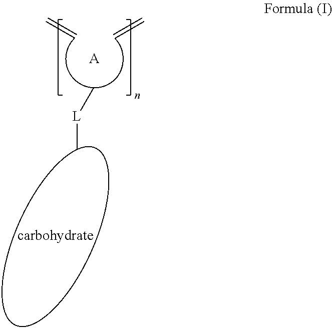

wherein: A represents a repeat unit selected from the group consisting of C₅-C₇ alkyl, C₅-C₇ heteroalkyl, C₅-C₇ aryl, C₅-C₇ heteroaryl, or C₅-C₇ cycloalkyl; n represents an integer from 10-500; L represents a divalent organic linker; and wherein L forms a direct bond to an anomeric position of the carbohydrate.

In one embodiment, the subject has cancer. In one embodiment, the composition suppresses proliferation of a cancer cell. In one embodiment, the composition inhibits tumor growth. In one embodiment, the composition inhibits at least one cancer cell from metastasizing. In one embodiment, the composition binds to a lectin. In one embodiment, the composition binds to a target protein selected from Gal-3, MGL, ASGPR, MBL, DC-SIGN, Langerin, GLUT-1, GLUT-2, GLUT-3, variants thereof, and combinations thereof.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1, comprising FIG. 1A through FIG. 1C, depicts the design and synthesis of carbohydrate-polymer conjugates (CPCs). FIG. 1A depicts the structure of galactose-based monomers (left) and lactose-based monomers (right). FIG. 1B depicts a general ring opening polymerization scheme of glycomonomers to afford CPCs in a single step. FIG. 1C depicts ¹H-NMR analysis of the polymerization of mono-Gal₇. Disappearance of the monomer alkene peak (orange) and appearance of two nonequivalent polymer alkene peaks (blue) indicate complete conversion FIG. 2, comprising FIG. 2A through FIG. 2C, depicts nuclear magnetic resonance (NMR) spectra of kinetics studies. FIG. 2A depicts representative kinetics ¹H NMR of mono-OH at various intervals. The blue box highlights the monomer alkene peak, the purple box highlights the polymer alkene peak, and the green box highlights the DMF-d₇ peak. FIG. 2B depicts representative kinetics ¹H NMR of mono-Gal₇ at various intervals. The blue box highlights the monomer alkene peak, the purple box highlights the polymer alkene peak, and the green box highlights the DMF-d₇ peak. FIG. 2C depicts representative kinetics ¹H NMR of mono-Lac₇ at various intervals. The blue box highlights the monomer alkene peak, the purple box highlights the polymer alkene peak, and the green box highlights the DMF-d₇ peak.

FIG. 3, comprising FIG. 3A through FIG. 3D, depicts size exclusion chromatography-multiangle light scattering (SEC-MALS) data. FIG. 3A depicts SEC-MALS data for polymerization kinetics experiments. FIG. 3B depicts SEC-MALS data for polymers used in microscale thermophoresis (MST) experiments. FIG. 3C depicts SEC-MALS data for polymers used to confirm computational analysis. FIG. 3D depicts SEC-MALS data for polymers used in biological experiments.

FIG. 4 depicts a general synthetic pathway to access glycomonomers. (i) TEA, Toluene, Reflux, 12 h. (ii) TBS-Cl, TEA, DCM, rt, 12 h. (iii) DCC, DMAP, 0° C. to rt, 12 h, 71%-77%. (iv) AcCl, dry MeOH, rt, 12 h, 67%-91%. (v) BF3·Et2O, dry DCM, 0° C. to rt, 12 h. (vi) NaOMe, MeOH, rt, 4 h, 31%-40%.

FIG. 5, comprising FIG. 5A and FIG. 5B, depicts the effect of glycan density on Galactin-3 (Gal-3) binding. FIG. 5A depicts a general chemical structure of copolymers with decreased galactose density. FIG. 5B depicts calculated K_{d} values for the Gal-3 binding of Gal-CPCs at DP=50 (light blue), 100 (medium blue), and 250 (dark blue), as well as Co-CPC-50% (light yellow) and Co-CPC-20% (dark yellow).

FIG. 6, comprising FIG. 6A through FIG. 6C, depicts Coarse-Grained Molecular Dynamics analyses. FIG. 6A depicts a representative schematic for the collapse of CPCs during the initial 50 ns of the simulation trajectory. The hydrophobic norbornene backbone is presented in red, whereas the carbohydrate functional groups are presented in cyan. FIG. 6B depicts a radial distribution function (RDF) between centers-of-mass (COMs) of attached monosaccharide groups on the CPCs, calculated for the last 150 ns of the simulation run. FIG. 6C depicts the evolution of the nonbonded interaction energy between monosaccharide groups and water throughout the simulation run.

FIG. 7, comprising FIG. 7A through FIG. 7C, depicts representative data over simulations. FIG. 7A depicts evolution of R_{g} of the entire CPC over the entire simulation run. FIG. 7B depicts R_{g} distribution for the entire CPC. FIG. 7C depicts R_{g} distribution for the backbone calculated for the last 150 ns of the simulation. All data is averaged over three simulations. Mean equilibrium values are included in the plots.

FIG. 8, comprising FIG. 8A through FIG. 8C, depicts radial distribution functions (RDFs). FIG. 8A depicts RDFs between water and COM of carbohydrate groups. FIG. 8B depicts RDFs between water and CPC backbone beads. FIG. 8C depicts RDFs between water and RDF between carbohydrate groups. Legends included in the plots.

FIG. 9, comprising FIG. 9A through FIG. 9C, depicts solvent accessible surface areas (SASA) for CPCs. FIG. 9A depicts evolution of SASA of the entire CPC over the entire simulation run. FIG. 9B depicts a SASA distribution for the CPC backbone. FIG. 9C depicts a SASA distribution for the CPC sidechains calculated for the last 150 ns of the simulation run. All plotted data is averaged over three simulation runs.

FIG. 10, comprising FIG. 10A through FIG. 10C, depicts evolution of nonbonded energy. FIG. 10A depicts evolution of nonbonded energy between CPC backbone and water. FIG. 10B depicts evolution of nonbonded energy between monosaccharide groups and water. FIG. 10C depicts evolution of nonbonded energy between monosaccharide groups, for the entire simulation run.

FIG. 11 depicts representative dynamic light scattering (DLS) traces of polymers disclosed in FIG. 3C and FIG. 3D.

FIG. 12 depicts a Galectin-3 western blot of 4T1 and LNCaP cells.

FIG. 13, comprising FIG. 13A through FIG. 13D, depicts interactions of CPCs with cancer cells. FIG. 13A depicts live-cell confocal images showing cell internalization of Gal-CPCs by 4T1 cells, following 4 h incubation. FIG. 13B depicts a plot of polymer cytotoxicity in Gal-3-negative LnCAP cells following 4 h incubation. FIG. 13C depicts a plot of polymer cytotoxicity in Gal-3-positive 4T1 cells following 4 h incubation. FIG. 13D depicts the effect of treatment on cancer cell proliferation, following 24 h incubation. Gal-CPCs effectively suppress cancer cell proliferation in gal-3-positive 4T1 cells. Statistical analysis was performed using an ordinary one-way ANOVA, where “**” represents a P value of <0.01, “***” represents a P value of <0.001, and “****” represents a P value of <0.0001.

FIG. 14, comprising FIG. 14A through FIG. 14C, depicts cell uptake and proliferation data. FIG. 14A depicts polymer cell uptake at 20× magnification. Top.) Fluorescence images of 4T1 cells incubated with Gal-7-100-Cy5 (λex=640 nm) and stained with CellBrite™ Steady 488 membrane stain (λex=488 nm). Bottom.) Fluorescence images of 4T1 cells treated only with membrane stain (λex=488 nm) and PBS. Scale bar set to 50 μm in the first and second columns and 100 μm in the overlayed column. FIG. 14B depicts large scale cell uptake via a 5×5 grid of 20× images. Top.) Fluorescence images of 4T1 cells incubated with Gal-7-100-Cy5 (λex=640 nm) and stained with CellBrite™ Steady 488 membrane stain (λex=488 nm). Bottom.) Fluorescence images of 4T1 cells treated only with membrane stain (λex=488 nm) and PBS. Scale bar set to 500 μm in the overlayed column. FIG. 14C depicts cell proliferation data using glucose polymer and the effect of treatment on LNCaP cell proliferation, following 24 h incubation. Statistical analysis was performed using an unpaired, two-tailed, t-test, where “****” represents a P value of <0.0001.

FIG. 15, comprising FIG. 15A through FIG. 15E, depicts antimigration activities of CPCs. FIG. 15A depicts representative images of scratched 4T1 cells before treatment (left) and after 24 h incubation with various constructs (right). Cells were stained with crystal violet to enhance contrast. FIG. 15B depicts all CPCs show dose-dependent inhibition of 4T1 cell migration. FIG. 15C depicts a comparison of antimigration efficacy of CPCs at 10 μM. At this dose, all constructs are able to suppress the migration of 4T1 cells; however, Gal-CPCs significantly outperform all other treatment groups. FIG. 15D depicts a comparison of antimigration efficacy of CPCs at 5 μM. FIG. 15E depicts a comparison of antimigration efficacy of CPCs at 2.5 μM.

FIG. 16 depicts chemical structures of galactose, mannose, and glucose monomers.

FIG. 17 depicts a general polymerization scheme.

FIG. 18 depicts polymerization kinetics monitored by nuclear magnetic resonance spectroscopy (NMR). All exo monomers were rapidly and completely polymerized while endo monomers converted slowly.

FIG. 19 depicts rate constant measurements for polymerizations.

FIG. 20 depicts polymerization kinetics by gel permeation chromatography (GPC) at 60° C. polymerization conditions. Mannose polymerization occurs significantly faster than glucose and galactose.

FIG. 21 depicts representative GPC analysis of polymers.

FIG. 22 depicts additional GPC analysis of polymers at lower degrees of polymerization (DPs).

FIG. 23 depicts circular dichroism (CD) spectra of all polymers in distilled water.

FIG. 24 depicts CD of Exo-PNP-Gal as a function of temperature.

FIG. 25 depicts a full CD sweep of Exo-PNP-Gal.

FIG. 26 depicts dynamic light scattering (DLS) data and CD (secondary structure) data of Exo-PNP-Gal and Exo-PNP-Glc.

FIG. 27 depicts dynamic light scattering (DLS) data and CD (secondary structure) data of Exo-PNP-Mannose.

FIG. 28 depicts Endo-Gal (left) and Endo-Glc (right) both exhibit the same behavior upon dialysis.

FIG. 29 depicts data which indicates that Endo-Man fails to form higher order assemblies in solution.

FIG. 30 depicts DLS data and transmission electron microscopy (TEM) images of Exo-Gal-NAs (nanoassemblies).

FIG. 31 depicts DLS data of Exo-Gal-NAs as a function of temperature.

FIG. 32 depicts representative TEM images of all nanoassemblies.

FIG. 33 depicts infrared (IR) spectra of Exo-Gal and Exo-Glc polymers.

FIG. 34 depicts DLS data of Exo-Gal and Endo-Gal, Exo-Glc and Endo-Glc, and Exo-Man and Endo-Man as a function of solvent.

FIG. 35 depicts Nile Red incorporation into the polymers.

FIG. 36 depicts DLS data and representative visual images of exo and endo polymers combined with Nile Red.

FIG. 37 depicts lectin (PNA) binding analysis of Gal polymers by DLS.

FIG. 38 depicts lectin (RCA120) binding analysis of Gal polymers by DLS

FIG. 39 depicts lectin (ConA) binding analysis of Glc polymers by DLS.

FIG. 40 depicts lectin (ConA) binding analysis of Gal polymers by DLS.

FIG. 41 depicts representative data of polymer-lectin binding as measured by microscale thermophoresis.

FIG. 42 depicts representative data of cell uptake of polymers as measured by flow cytometry.

FIG. 43 depicts a representative summary of the present invention.

FIG. 44 depicts the upregulation of Gal-3 in cancer and other inflammatory diseases.

FIG. 45 depicts the role of Gal-3 in anti-cancer immunity.

FIG. 46 depicts a schematic describing the goal to develop CPCs that bind Gal-3 with high avidity and specificity with the hypothesis that the macromolecular structure and presentation of carbohydrates dictates the avidity of galectin-3 binding.

FIG. 47 depicts representative monomers.

FIG. 48 depicts a representative scheme for ROMP.

FIG. 49 depicts polymerization results of galactose containing glycomonomers.

FIG. 50 depicts polymerization results of lactose containing glycomonomers.

FIG. 51 depicts representative data which shows the acetylation of lactose enables higher DP polymerizations.

FIG. 52 depicts representative data which shows how Galactose-CPCs bind Gal-3 with micromolar affinity. The number of atoms between galactose and the polymer backbone significantly influences binding to Gal-3.

FIG. 53 depicts representative derivatives of monomers.

FIG. 54 depicts representative data displaying the difference in behavior between Endo-CPCs and Exo-CPCs.

FIG. 55 depicts a general synthetic pathway to access glycomonomer. (i) Benzaldehyde dimethyl acetal, CSA, ACN, rt, 12 h, 88% (ii) BzCl, pyridine, rt, 12 h, 91% (iii) TEA, Toluene, Reflux, 12 h, 87%. (iv) NIS, TMSOTf, dry DCM, −40° C., 2 h, 67%. (v) DTT, CSA, dry DCM, rt, 7 h. (vi) NaOMe, MeOH, rt, 2 h, 62% (2 steps).

FIG. 56, comprising FIG. 56A through FIG. 56C, depicts course-grain (CG) mapping schemes. FIG. 56A depicts a CG mapping scheme for Co-CPC-50%. FIG. 56B depicts a CG mapping scheme for Gal-CPC. FIG. 56C depicts a CG mapping scheme for Lac-CPC.

FIG. 57, comprising FIG. 57A through FIG. 57A, depicts representative initial configurations. FIG. 57A depicts a representative initial configuration for Co-CPC-50%. FIG. 57B depicts a representative initial configuration for Gal-CPC. FIG. 57C depicts a representative initial configuration for Lac-CPC.

FIG. 58 depicts distributions of bond lengths obtained from CG MD simulations of 3-galactose and β-glucose, and their corresponding mapped AA trajectories

FIG. 59 depicts distributions of angles obtained from CG MD simulations of β-galactose and β-glucose, and their corresponding AA trajectories.

FIG. 60, comprising FIG. 60A and FIG. 60B, depicts additional modeling for CPC systems. FIG. 60A depicts representative final configurations for Co-CPC-50%, Gal-CPC, and Lac-CPC. FIG. 60B depicts dihedral angle autocorrelation functions calculated for two major CPC backbone dihedrals. Legends included in the plots.

### DETAILED DESCRIPTION

The present invention provides compositions and methods for the synthesis of a carbohydrate-polymer conjugate. In one embodiment, the invention provides a carbohydrate-polymer conjugate comprising a ring comprising an olefin, a divalent linking group, and a carbohydrate. In one embodiment, the invention provides a method of synthesizing the carbohydrate-polymer conjugate. In one embodiment, the invention provides a method of use of the carbohydrate-polymer conjugate.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

As used herein, the term “organic” includes polymeric materials as well as small molecule organic materials. “Small molecule” refers to any organic material that is not a polymer, and “small molecules” may actually be quite large. Small molecules may include repeat units in some circumstances. For example, using a long chain alkyl group as a substituent does not remove a molecule from the “small molecule” class. Small molecules may also be incorporated into polymers, for example as a pendent group on a polymer backbone or as a part of the backbone. Small molecules may also serve as the core moiety of a dendrimer, which consists of a series of chemical shells built on the core moiety.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and including straight, branched chain, or cyclic substituent groups. Examples include methyl, ethyl, propyl, isopropyl, butyl, isobutyl, tert-butyl, pentyl, neopentyl, hexyl, and cyclopropylmethyl.

As used herein, the term “substituted alkyl” means alkyl as defined above, substituted by one, two or three substituents selected from the group consisting of halogen, —OH, alkoxy, —NH₂, amino, azido, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂. Examples of substituted alkyls include, but are not limited to, 2,2-difluoropropyl, 2-carboxycyclopentyl and 3-chloropropyl.

As used herein, the term “olefin” encompasses compounds having a C═C bond.

The term “olefin-based polymer,” as used herein, refers to a polymer that contains at least a majority weight percent, based on the weight of the polymer, polymerized olefin (for example, ethylene or propylene), and, optionally, one or more additional comonomers.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers.

As used herein, the term “halo” or “halogen” alone or as part of another substituent means, unless otherwise stated, a fluorine, chlorine, bromine, or iodine atom.

As used herein, the term “cycloalkyl” refers to a mono cyclic or polycyclic non-aromatic radical, wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following moieties:

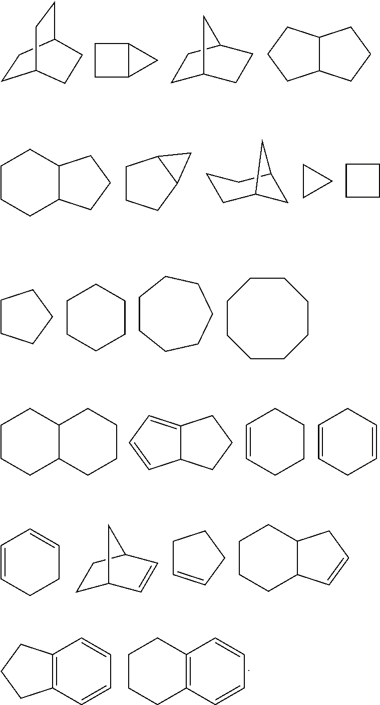

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon double bond or one carbon triple bond.

As used herein, the term “heterocycloalkyl” or “heterocyclyl” or “heterocyclic” refers to a cyclic group containing one to four ring heteroatoms each selected from O, S, and N. In one embodiment, each heterocycloalkyl group has from 4 to 10 atoms in its ring system, with the proviso that the ring of said group does not contain two adjacent O atoms. In another embodiment, the heterocycloalkyl group is fused with an aromatic ring. In one embodiment, the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine.

Examples of non-aromatic heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl.

As used herein, the term “aryl-(C₁-C₃)alkyl” means a functional group wherein a one- to three-carbon alkylene chain is attached to an aryl group, e.g., —CH₂CH₂— phenyl, —CH₂-phenyl (benzyl), aryl-CH₂— and aryl-CH(CH₃)—. The term “substituted aryl-(C₁-C₃)alkyl” means an aryl-(C₁-C₃)alkyl functional group in which the aryl group is substituted. Similarly, the term “heteroaryl-(C₁-C₃)alkyl” means a functional group wherein a one to three carbon alkylene chain is attached to a heteroaryl group, e.g., —CH₂CH₂-pyridyl. The term “substituted heteroaryl-(C₁-C₃)alkyl” means a heteroaryl-(C₁-C₃)alkyl functional group in which the heteroaryl group is substituted.

As used herein, the term “heteroaryl” or “heteroaromatic” refers to aryl groups which contain at least one heteroatom selected from N, O, Si, P, and S; wherein the nitrogen and sulfur atoms may be optionally oxidized, and the nitrogen atom(s) may be optionally quaternized. Heteroaryl groups may be substituted or unsubstituted. A heteroaryl group may be attached to the remainder of the molecule through a heteroatom. A polycyclic heteroaryl may include one or more rings that are partially saturated. Examples include tetrahydroquinoline, 2,3-dihydrobenzofuryl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups also include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles and heteroaryls include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, S(═O)₂N[H, alkyl, or aryl], —C(═O)NH[substituted or unsubstituted alkyl, or substituted or unsubstituted phenyl], —C(═O)N[H or substituted or unsubstituted alkyl or aryl]2, —OC(═O)N[substituted or unsubstituted alkyl]₂, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]₂, and —C(NH₂)[substituted or unsubstituted alkyl]₂. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, —ON(O)₂, and —C(═O)OH. In yet one embodiment, the substituents are independently selected from the group consisting of C₁₋₆ alkyl, —OH, C₁₋₆ alkoxy, halo, amino, acetamido, oxo and nitro. As used herein, where a substituent is an alkyl or alkoxy group, the carbon chain may be branched, straight or cyclic.

As used herein, the term “protected,” as used herein, refers to the presence of a “protecting group” or moiety that prevents reaction of the chemically reactive functional group under certain reaction conditions. The protecting group will vary depending on the type of chemically reactive group being protected. By way of example only, (i) if the chemically reactive group is an amine or a hydrazide, the protecting group may be selected from tert-butyloxycarbonyl (t-Boc) and 9-fluorenylmethoxycarbonyl (Fmoc); (ii) if the chemically reactive group is a thiol, the protecting group may be orthopyridyldisulfide; and (iii) if the chemically reactive group is a carboxylic acid, such as butanoic or propionic acid, or a hydroxyl group, the protecting group may be benzyl or an alkyl group such as methyl, ethyl, or tert-butyl. Additionally, protecting groups include, but are not limited to, photolabile groups, such as Nvoc and MeNvoc, and other protecting groups known in the art. Other protecting groups are described in Greene and Wuts, Protective Groups in Organic Synthesis, 3rd Ed., John Wiley & Sons, New York, N.Y., 1999.

The term “derivative” refers to a small molecule that differs in structure from the reference molecule but retains the essential properties of the reference molecule. A derivative may change its interaction with certain other molecules relative to the reference molecule. A derivative molecule may also include a salt, an adduct, tautomer, isomer, or other variant of the reference molecule.

The term “tautomers” are constitutional isomers of organic compounds that readily interconvert by a chemical process (tautomerization).

The term “isomers” or “stereoisomers” refer to compounds, which have identical chemical constitution, but differ with regard to the arrangement of the atoms or groups in space.

As used herein, the term “polymer” refers to a molecule composed of repeating structural units typically connected by covalent chemical bonds. The term “polymer” is also meant to include the terms copolymer and oligomers.

As used herein, the term “polymerization” refers to at least one reaction that consumes at least one functional group in a monomeric molecule (or monomer), oligomeric molecule (or oligomer) or polymeric molecule (or polymer), to create at least one chemical linkage between at least two distinct molecules (e.g., intermolecular bond), at least one chemical linkage within the same molecule (e.g., intramolecular bond), or any combination thereof. A polymerization reaction may consume between about 0% and about 100% of the at least one functional group available in the system. In one embodiment, polymerization of at least one functional group results in about 100% consumption of the at least one functional group. In another embodiment, polymerization of at least one functional group results in less than about 100% consumption of the at least one functional group.

As used herein, the term “polymer segment” means and includes a grouping of multiple monomer units of a single type (i.e., a homopolymer segment) or multiple types (i.e., a copolymer segment) of constitutional units into a continuous region of a polymer block that are of a length that is insufficient for microphase separation to inherently occur with other segments in the same block type.

As used herein, the term “block copolymer” means and includes a polymer composed of chains where each chain contains two or more polymer blocks as defined above and at least two of the blocks are of sufficient segregation strength (e.g., χN>10) for those blocks to phase separate. A wide variety of block polymers are contemplated herein including diblock copolymers (i.e., polymers including two polymer blocks), triblock copolymers (i.e., polymers including three polymer blocks), multiblock copolymers (i.e., polymers including more than three polymer blocks), and combinations thereof.

As used herein, a “disease” is a state of health of an animal wherein the animal cannot maintain homeostasis, and wherein if the disease is not ameliorated then the animal's health continues to deteriorate.

As used herein, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein with respect to the compositions of the invention, “biologically active” means that the compositions elicit a biological response in a mammal that can be monitored and characterized in comparison with an untreated mammal.

As used herein, the term “treating” means ameliorating the effects of, or delaying, halting or reversing the progress of a disease or disorder. The word encompasses reducing the severity of a symptom of a disease or disorder and/or the frequency of a symptom of a disease or disorder.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease. Disease and disorder are used interchangeably herein.

As used herein, the term “medical intervention” means a set of one or more medical procedures or treatments that are required for ameliorating the effects of, delaying, halting or reversing a disease or disorder of a subject. A medical intervention may involve surgical procedures or not, depending on the disease or disorder in question. A medical intervention may be wholly or partially performed by a medical specialist, or may be wholly or partially performed by the subject himself or herself, if capable, under the supervision of a medical specialist or according to literature or protocols provided by the medical specialist.

As used herein, the terms “effective amount” or “therapeutically effective amount” or “pharmaceutically effective amount” of a composition are used interchangeably to refer to the amount of the composition that is sufficient to provide a beneficial effect to the subject to which the composition is administered. The term to “treat,” as used herein, means reducing the frequency with which symptoms are experienced by a patient or subject or administering a composition to reduce the severity with which symptoms are experienced. An appropriate therapeutic amount in any individual case may be determined by one of ordinary skill in the art using routine experimentation.

By the term “specifically bind” or “specifically binds,” as used herein, is meant that a first molecule (e.g., an antibody) preferentially binds to a second molecule (e.g., a particular antigenic epitope), but does not necessarily bind only to that second molecule.

As used herein, a “prophylactic” or “preventive” treatment is a treatment administered to a subject who does not exhibit signs of a disease or disorder or exhibits only early signs of the disease or disorder for the purpose of decreasing the risk of developing pathology associated with the disease or disorder.

As used herein, a “therapeutic” treatment is a treatment administered to a subject who exhibits signs of pathology of a disease or disorder for the purpose of diminishing or eliminating those signs.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, a “pharmaceutically acceptable carrier” means a pharmaceutically acceptable material, composition or carrier, such as a liquid or solid filler, diluent, excipient, solvent or encapsulating material, involved in carrying or transporting a compound(s) of the present invention within or to the subject such that it can perform its intended function. Typically, such compounds are carried or transported from one organ, or portion of the body, to another organ, or portion of the body. Each carrier must be “acceptable” in the sense of being compatible with the other ingredients of the formulation, and not injurious to the patient. Some examples of materials that can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. As used herein “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds can also be incorporated into the compositions.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

As used herein, the term “subject” refers to a human or another mammal (e.g., primate, dog, cat, goat, horse, pig, mouse, rat, rabbit, and the like) that can have a disease, disorder, or condition; or be at risk for developing a disease, disorder, or condition; but may or may not have a disease, disorder, or condition or be at risk for developing a disease, disorder, or condition. In many embodiments of the present invention, the subject is a human being. In such embodiments, the subject is often referred to as an “individual” or a “patient.” The terms “individual” and “patient” do not denote a particular age.

As used here, “biocompatible” refers to any material, which, when implanted in a mammal, does not provoke an adverse response in the mammal. A biocompatible material, when introduced into an individual, is not toxic or injurious to that individual, nor does it induce immunological rejection of the material in the mammal.

The term “biodegradable” includes polymers, compositions and formulations, such as those described herein, that are intended to degrade during use. Biodegradable polymers typically differ from non-biodegradable polymers in that the former may be degraded during use. In one embodiment, such use involves in vivo use, such as in vivo therapy. In another embodiment, such use involves in vitro use. In general, biodegradation involves the degradation of a biodegradable polymer into its component subunits, or digestion, e.g., by a biochemical process, of the polymer into smaller, non-polymeric subunits. Two types of biodegradation may generally be identified. For example, biodegradation may involve cleavage of bonds (whether covalent or otherwise) in the polymer backbone. In such biodegradation, monomers and oligomers typically result, and even more typically, such biodegradation occurs by cleavage of a bond connecting one or more of subunits of a polymer. Further, biodegradation may involve cleavage of a bond (whether covalent or otherwise) internal to side chain or that connects a side chain to the polymer backbone. For example, a therapeutic agent or other chemical moiety attached as a side chain to the polymer backbone may be released by biodegradation. In one embodiment, at least one type of biodegradation may occur during use of a polymer. As used herein, the term “biodegradation” encompasses all known types of biodegradation.

As used herein, the terms “biocompatible polymer” and “biocompatibility” when used in relation to polymers are recognized in the art. For example, biocompatible polymers include polymers that are generally neither toxic to the host, nor degrade (if the polymer degrades) at a rate that produces monomeric or oligomeric subunits or other byproducts at toxic concentrations in the host. In one embodiment, biodegradation generally involves degradation of the polymer in a host, e.g., into its monomeric subunits, which may be known to be effectively non-toxic. Intermediate oligomeric products resulting from such degradation may have different toxicological properties, however, or biodegradation may involve oxidation or other biochemical reactions that generate molecules other than monomeric subunits of the polymer. Consequently, in one embodiment, toxicology of a biodegradable polymer intended for in vivo use, such as implantation or injection into a patient, may be determined after one or more toxicity analyses. It is not necessary that any subject composition have a purity of 100% to be deemed biocompatible; indeed, it is only necessary that the subject compositions be biocompatible as set forth above. Hence, a subject composition may comprise polymers comprising 99%, 98%, 97%, 96%, 95%, 90%, 85%, 80%, 75% or even less of biocompatible polymers, e.g., including polymers and other materials and excipients described herein, and still be biocompatible.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, head and neck cancers, lymphoma, leukemia, lung cancer and the like.

As used herein, the phrase “a tumor site” refers to any site or region within a subject which a tumor has formed, may be expected to form, or was previously located. In certain embodiments, the tumor site is in need of anti-tumor activity.

Several references to integers and R, R¹, R², R³, R⁴, R⁵, R⁶, etc. are made in chemical structures and moieties disclosed and described herein. Any description of integers and R, R¹, R², R³, R⁴, R⁵, R⁶, etc. in the specification is applicable to any structure or moiety reciting integers and R, R¹, R², R³, R⁴, R⁵, R⁶, etc. respectively.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compositions

The present invention is drawn to, in part, a carbohydrate-polymer conjugate comprising a structure represented by Formula (I):

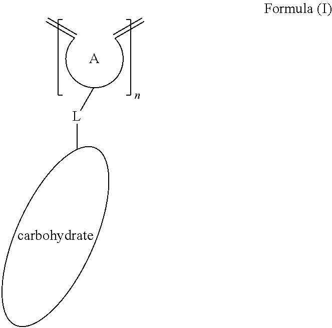

wherein:
A represents a repeat unit selected from the group consisting of C₅-C₇ alkyl, C₅-C₇ heteroalkyl, C₅-C₇ aryl, C₅-C₇ heteroaryl, or C₅-C₇ cycloalkyl; n represents an integer from 10-500; L represents a divalent organic linker; and wherein L forms a direct bond to an anomeric position of the carbohydrate.

In one embodiment, ring A is represented by one of the following structures:

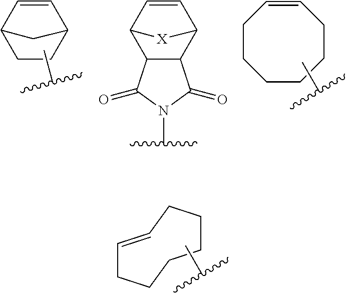

wherein the wave line represents a bond to L; X represents O, S, NR¹, or CR²R³; wherein R¹, R², and R³ each independently represent a substituent selected from the group consisting of hydrogen, halide, hydroxy, C₁-C₃₀ alkyl, C₁-C₃₀ alkyl halide, C₁-C₃₀ alkoxy, and any combination thereof, and wherein each structure is independently optionally further substituted. In one embodiment, ring A comprises a cycloalkyl. In one embodiment, ring A comprises a heterocycloalkyl. In one embodiment, ring A comprises an aryl. In one embodiment, ring A comprises at least one olefin. In one embodiment, ring A is further substituted. In one embodiment, the carbohydrate-polymer conjugate is represented by Formula (I).

In one embodiment, ring A comprises a norbornene. In one embodiment, ring A comprises an olefin that is more reactive than norbornene. In one embodiment, ring A is in an endo-configuration. In one embodiment, ring A is in an exo-configuration. In one embodiment, ring A is a racemate.

As used herein, the terms “endo-” and “exo-” define isomerism of organic compounds comprising a substituent in a bridged ring system. The prefix “endo” or a compound or moiety in an “endo-configuration” is defined to have the highest priority substituent closest, or “syn” to the longest bridge, whereas “exo” or a compound or moiety in an “exo-configuration” is defined to have the highest priority substituent close, or “anti” to the longest bridge.

In one embodiment, the polymer backbone is unsaturated. In one embodiment, the polymer backbone is rigid. In one embodiment, the polymer backbone is partially saturated. In one embodiment, the polymer backbone is saturated. In one embodiment, the polymer backbone is flexible. In one embodiment, the polymer backbone comprises one or more pendant hydroxyl groups. In one embodiment, the polymer backbone is functionalizable. In one embodiment, the polymer backbone has an end group comprising a compound selected from the group consisting of a fluorophore, a terminal alkene, a benzyl group, an amino group, a therapeutic small molecule, and combinations thereof.

In one embodiment, the linking group L is represented by one of the following structures:

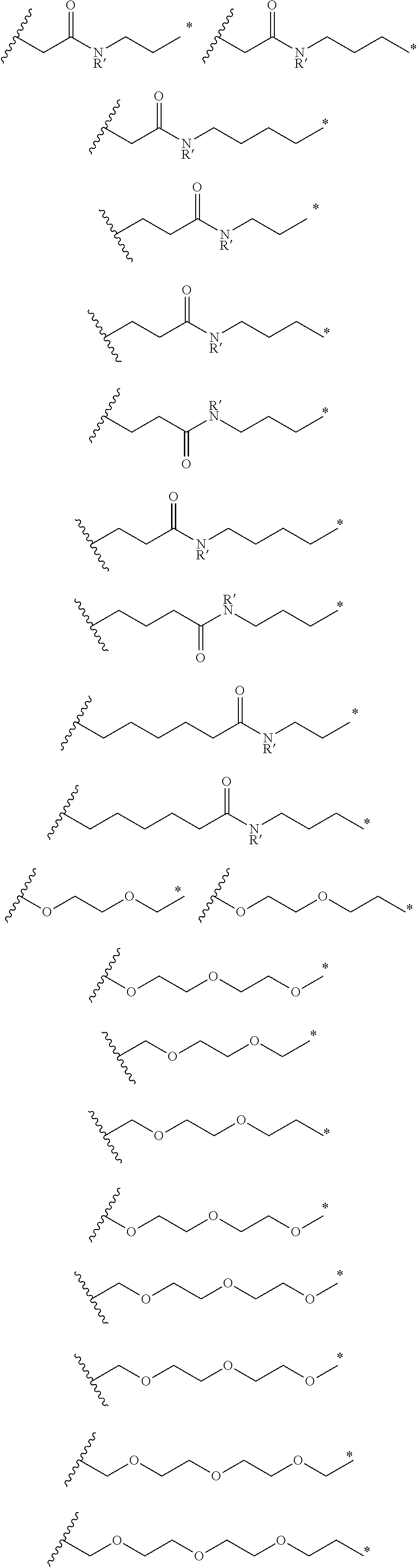

wherein the wavy line represents a bond to ring A; * represents a bond to the carbohydrate; and R′ represents a substituent selected from the group consisting of hydrogen, halide, hydroxy, C₁-C₃₀ alkyl, C₁-C₃₀ alkyl halide, C₁-C₃₀ alkoxy, and any combination thereof. In one embodiment, L comprises a C₁-C₃₀ alkyl which is optionally further substituted.

In one embodiment, the linker comprises an amino acid. In one embodiment, the linker comprises a glycol. In one embodiment, the linker is capable of hydrogen-bonding. In one embodiment, R′ is hydrogen. In one embodiment, R′ is an alkoxy group. In one embodiment, R′ participates in hydrogen bonding. In one embodiment, R′ participates in intramolecular hydrogen bonding. In one embodiment, R′ participates in intermolecular hydrogen bonding. In one embodiment, the linker comprises a C₁-C₃₀ alkyl.

In one embodiment, the carbohydrate is selected from the group consisting of a monosaccharide, a disaccharide, an oligosaccharide, a polysaccharide, any stereochemical isomer thereof, and any combination thereof. Exemplary carbohydrates include, but are not limited to, monosaccharides including trioses (such as: ketotriose (dihydroxyacetone); aldotriose (glyceraldehyde)); tetroses which include: ketotetrose (such as: erythrulose) and aldotetroses (such as: erythrose, threose); pentoses which include: ketopentose (such as: ribulose, xylulose) aldopentose (such as: ribose, arabinose, xylose, lyxose), deoxy sugar (such as: deoxyribose); hexoses which include: ketohexose (such as: psicose, fructose, sorbose, tagatose), aldohexose (such as: allose, altrose, glucose, mannose, gulose, idose, galactose, talose), deoxy sugar (such as: fucose, fuculose, rhamnose); heptose (such as: sedoheptulose); octose; nonose (such as: neuraminic acid); disaccharides which include: sucrose; lactose; maltose; trehalose; turanose; cellobiose; kojiboise; nigerose; isomaltose; and palatinose; trisaccharides which include: melezitose; and maltotriose; oligosaccharides that include: corn syrups and maltodextrin; and polysaccharides that include: glucan (such as dextrin, dextran, beta-glucan), glycogen, mannan, galactan, and starch (such as those from corn, wheat, tapioca, rice, and potato, including amylose and amylopectin. The starches can be natural or modified or gelatinized); or combinations thereof. Carbohydrates also include source of sweeteners such as honey, maple syrup, glucose (dextrose), corn syrup, corn syrup solids, high fructose corn syrups, crystalline fructose, juice concentrates, dextrose polymers, malt syrup, rice syrup solids, sorghum syrup, refiner syrup, crystalline fructose, brown or invert sugars, molasses, or other grain/nut syrups consisting of rice syrup, agave syrup, palm syrup, and crystalline juice.

In one embodiment, the carbohydrate is selected from the group consisting of galactose, glucose, mannose, allose, altrose, gulose, idose, talose, psicose, fructose, sorbose, tagatose, and any stereochemical isomer thereof. In one embodiment, the carbohydrate comprises galactose.

In one embodiment, the carbohydrate comprises glucose. In one embodiment, the carbohydrate comprises mannose. In one embodiment, the carbohydrate is stereochemically pure. In one embodiment, the carbohydrate is a racemate. In one embodiment, the carbohydrate is partially stereochemically pure. In one embodiment, the carbohydrate is derived from commercially available sources. In one embodiment, the carbohydrate is extracted from food.

In one embodiment, the carbohydrate-polymer conjugate is a copolymer comprising a repeat unit of at least two monomers, wherein at least one monomer comprises galactose. In one embodiment, the carbohydrate-polymer conjugate is a block copolymer. Other exemplary copolymers which may describe the present invention include, but are not limited to, random copolymers, statistical copolymers, alternating copolymers, stereoblock copolymers, gradient copolymers, graft copolymers, star copolymers, and combinations thereof. In one embodiment, the carbohydrate-polymer conjugate comprises a monomer which does not comprise a carbohydrate. In one embodiment, the carbohydrate-polymer conjugate comprises a monomer which comprises a hydroxyl capped linker. In one embodiment, the carbohydrate-polymer conjugate comprises a monomer which comprises a linker bonded to a protecting group. In one embodiment, the carbohydrate is acetylated.

In one embodiment, the carbohydrate-polymer conjugate forms higher order assemblies. In one embodiment, the carbohydrate-polymer conjugate forms secondary structures. In one embodiment, the carbohydrate-polymer conjugate comprises exposed hydrophobic pockets. In one embodiment, the carbohydrate-polymer conjugate forms nanoassemblies. In one embodiment, the carbohydrate-polymer conjugate forms higher order assemblies after dialysis. In one embodiment, the carbohydrate-polymer conjugate forms higher order assemblies after dialysis with DMSO. In one embodiment, the carbohydrate-polymer conjugate participates in intramolecular hydrogen bonding. In one embodiment, the carbohydrate-polymer conjugate participates in intermolecular hydrogen bonding.

In one embodiment, the carbohydrate-polymer conjugate readily binds a protein target. In one embodiment, the carbohydrate-polymer conjugate readily forms micelles. In one embodiment, the carbohydrate-polymer conjugate readily forms micelles with hydrophobic dyes. In one embodiment, the carbohydrate-polymer conjugate is biocompatible. In one embodiment, the carbohydrate-polymer conjugate binds a target which is overexpressed in cancer. In one embodiment, the carbohydrate-polymer conjugate is a biomimetic.

In one embodiment, the carbohydrate-polymer conjugate has a grafting density of about 5% to about 100%. In one embodiment, the carbohydrate-polymer conjugate has a grafting density of about 10% to about 100%. In one embodiment, the carbohydrate-polymer conjugate has a grafting density of about 15% to about 100%. In one embodiment, the carbohydrate-polymer conjugate has a graft density of about 20% to about 100%. In one embodiment, the carbohydrate-polymer conjugate has a grafting density of about 30% to about 100%. In one embodiment, the carbohydrate-polymer conjugate has a graft density of about 40% to about 100%. In one embodiment, the carbohydrate-polymer conjugate has a grafting density of about 50% to about 100%. In one embodiment, the carbohydrate-polymer conjugate has a graft density of about 60% to about 100%. In one embodiment, the carbohydrate-polymer conjugate has a grafting density of about 70% to about 100%. In one embodiment, the carbohydrate-polymer conjugate has a graft density of about 80% to about 100%. In one embodiment, the carbohydrate-polymer conjugate has a grafting density of about 90% to about 100%. In one embodiment, the carbohydrate-polymer conjugate has a grafting density of about 10% to about 90%. In one embodiment, the carbohydrate-polymer conjugate has a graft density of about 20% to about 90%. In one embodiment, the carbohydrate-polymer conjugate has a grafting density of about 30% to about 90%. In one embodiment, the carbohydrate-polymer conjugate has a graft density of about 40% to about 90%. In one embodiment, the carbohydrate-polymer conjugate has a grafting density of about 50% to about 90%. In one embodiment, the carbohydrate-polymer conjugate has a graft density of about 60% to about 90%. In one embodiment, the carbohydrate-polymer conjugate has a grafting density of about 70% to about 90%. In one embodiment, the carbohydrate-polymer conjugate has a graft density of about 80% to about 90%.

In one embodiment, the carbohydrate-polymer conjugate is readily dissolved in a biorelevant medium. In one embodiment, the carbohydrate-polymer conjugate readily forms a suspension in a biorelevant medium.

The present invention is further drawn to, in part, compositions and formulations comprising the carbohydrate-polymer conjugate. In one embodiment, the composition is a biodegradable composition. In one embodiment, the composition is a medical biodegradable composition.

In various aspects, the composition comprises: one or more polymers of the present invention and one or more stabilizers. In other aspects, the composition comprises: one or more nanoparticles of the present invention and one or more stabilizers. In various embodiments, the stabilizer to nanoparticle weight ratio is less than 50%. In one embodiment, the stabilizer comprises a biocompatible polymer. Examples of stabilizers include, but are not limited to, biocompatible polymer, a biodegradable polymer, a multifunctional linker, starch, modified starch, and starch derivatives, gums, including but not limited to polymers, polypeptides, albumin, amino acids, thiols, amines, carboxylic acid and combinations or derivatives thereof, citric acid, xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum, polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(imides), poly(benzimidazoles), collodion, regenerated proteins, semi-solid cross-linked poly(vinylpyrrolidone), monomeric, dimeric, oligomeric or long-chain, copolymers, block polymers, block co-polymers, polymers, PEG, dextran, modified dextran, polyvinylalcohol, and polyvinylpyrollidone.

The compositions are formulated in a pharmaceutically acceptable excipient, such as wetting agents, buffers, disintegrants, binders, fillers, flavoring agents and liquid carrier media such as sterile water, water/ethanol etc. The compositions should be suitable for administration either by topical administration or injection or inhalation or catheterization or instillation or transdermal introduction into any of the various body cavities including the alimentary canal, the vagina, the rectum, the bladder, the ureter, the urethra, the mouth, etc. For oral administration, the pH of the composition is preferably in the acid range (e.g., 2 to 7) and buffers or pH adjusting agents may be used. The contrast media may be formulated in conventional pharmaceutical administration forms, such as tablets, capsules, powders, solutions, dispersion, syrups, suppositories etc.

The compounds, nanoparticles, or compositions of the invention can be formulated and administered to a subject, as now described. The invention encompasses the preparation and use of pharmaceutical compositions comprising the compound, nanoparticle, and/or compositions of the invention useful for the delivery of a therapeutic agent to a cell. The invention also encompasses the preparation and use of pharmaceutical compositions comprising the compound, nanoparticle, and/or compositions of the invention useful for the treatment of a disease or disorder.

Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

The pharmaceutical compositions useful for practicing the invention may be administered to deliver a dose of between about 0.01 ng/kg/day and 500 mg/kg/day.

In various embodiments, the pharmaceutical compositions useful in the methods of the invention may be administered, by way of example, systemically, parenterally, or topically, such as, in oral formulations, inhaled formulations, including solid or aerosol, and by topical or other similar formulations. In addition to the appropriate therapeutic composition, such pharmaceutical compositions may contain pharmaceutically acceptable carriers and other ingredients known to enhance and facilitate drug administration. Other possible formulations, such as nanoparticles, liposomes, resealed erythrocytes, and immunologically based systems may also be used to administer an appropriate modulator thereof, according to the methods of the invention.

Although the descriptions of pharmaceutical compositions provided herein are principally directed to pharmaceutical compositions which are suitable for ethical administration to humans, it will be understood by the skilled artisan that such compositions are generally suitable for administration to animals, patients, and subjects of all sorts. Modification of pharmaceutical compositions suitable for administration to humans in order to render the compositions suitable for administration to various animals and patients is well understood, and the ordinarily skilled veterinary pharmacologist can design and perform such modification with merely ordinary, if any, experimentation.

Pharmaceutical compositions that are useful in the methods of the invention may be prepared, packaged, or sold in formulations suitable for oral, rectal, vaginal, parenteral, topical, pulmonary, intranasal, buccal, intravenous, ophthalmic, intrathecal and other known routes of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture.

Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent.

Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, cutaneous, subcutaneous, intraperitoneal, intravenous, intramuscular, intracisternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers.

Such a formulation is administered in the manner in which snuff is taken i.e. by rapid inhalation through the nasal passage from a container of the powder held close to the nares. Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, contain 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 nanometers to about 2000 micrometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa.

Typically dosages of the compound of the invention which may be administered to an animal or patient, preferably a human, range in amount from about 0.01 mg to about 100 g per kilogram of body weight of the animal or patient. While the precise dosage administered will vary depending upon any number of factors, including, but not limited to, the type of animal and type of disease state being treated, the age of the animal or patient and the route of administration. Preferably, the dosage of the compound will vary from about 0.01 mg to about 500 mg per kilogram of body weight of the animal or patient. The compound can be administered to an animal or patient as frequently as several times daily, or it can be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, patient, etc.

Administration of the compounds of the present invention or the compositions thereof may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the mammal, and whether prevention or treatment is to be achieved. Such factors can be readily determined by the clinician employing animal models or other test systems which are well known to the art.

One or more suitable unit dosage forms having the therapeutic agent(s) of the invention, which, as discussed below, may optionally be formulated for sustained release, can be administered by a variety of routes including parenteral, including by intravenous and intramuscular routes, as well as by direct injection into the diseased tissue. For example, the therapeutic agent may be directly injected into the muscle. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium Ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The active ingredients of the invention may be formulated to be suspended in a pharmaceutically acceptable composition suitable for use in mammals and in particular, in humans. Such formulations include the use of adjuvants such as muramyl dipeptide derivatives (MDP) or analogs that are described in U.S. Pat. Nos. 4,082,735; 4,082,736; 4,101,536; 4,185,089; 4,235,771; and 4,406,890. Other adjuvants, which are useful, include alum (Pierce Chemical Co.), lipid A, trehalose dimycolate and dimethyldioctadecylammonium bromide (DDA), Freund's adjuvant, and IL-12. Other components may include a polyoxypropylene-polyoxyethylene block polymer (Pluronic®), a non-ionic surfactant, and a metabolizable oil such as squalene (U.S. Pat. No. 4,606,918).

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly(lactic acid) or ethylenevinylacetate copolymers. In addition to being incorporated, these agents can also be used to trap the compound in microcapsules.

Accordingly, the composition of the present invention may be delivered via various routes and to various sites in a mammal body to achieve a particular effect (see, e.g., Rosenfeld et al., 1991; Rosenfeld et al., 1991a; Jaffe et al., supra; Berkner, supra). One skilled in the art will recognize that although more than one route can be used for administration, a particular route can provide a more immediate and more effective reaction than another route. In one embodiment, the composition described above is administered to the subject by subretinal injection. In other embodiments, the composition is administered by intravitreal injection. Other forms of administration that may be useful in the methods described herein include, but are not limited to, direct delivery to a desired organ (e.g., the eye), oral, inhalation, intranasal, intratracheal, intravenous, intramuscular, subcutaneous, intradermal, and other parental routes of administration. Additionally, routes of administration may be combined, if desired. In another embodiments, route of administration is subretinal injection or intravitreal injection.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the composition in the particular host.

These methods described herein are by no means all-inclusive, and further methods to suit the specific application will be apparent to the ordinary skilled artisan. Moreover, the effective amount of the compositions can be further approximated through analogy to compounds known to exert the desired effect.

It will be understood by those of skill in the art that numerous and various modifications can be made without departing from the spirit of the present disclosure. Therefore, it should be clearly understood that the forms disclosed herein are illustrative only and are not intended to limit the scope of the present disclosure.

### Methods of Making

The present invention is further drawn to, in part, a method of synthesizing a carbohydrate-polymer conjugate using ring-opening metathesis polymerization (ROMP), comprising the steps of: providing a monomer; polymerizing the monomer using a ROMP catalyst; and isolating the carbohydrate-polymer conjugate; wherein the monomer is represented

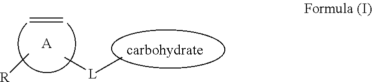

wherein: R represents a substituent on ring A selected from the group consisting of hydrogen, halide, hydroxy, C₁-C₃₀ alkyl, C₁-C₃₀ alkyl halide, C₁-C₃₀ alkoxy, and any combination thereof, and L represents a divalent organic linker.

In one embodiment, polymerization is performed with a target degree of polymerization. In one embodiment, the degree of polymerization is about 10 to about 500. In one embodiment, the degree of polymerization is about 20 to about 500. In one embodiment, the degree of polymerization is about 30 to about 500. In one embodiment, the degree of polymerization is about 40 to about 500. In one embodiment, the degree of polymerization is about 50 to about 500. In one embodiment, the degree of polymerization is about 60 to about 500. In one embodiment, the degree of polymerization is about 70 to about 500. In one embodiment, the degree of polymerization is about 80 to about 500. In one embodiment, the degree of polymerization is about 90 to about 500. In one embodiment, the degree of polymerization is about 100 to about 500. In one embodiment, the degree of polymerization is about 10 to about 400. In one embodiment, the degree of polymerization is about 20 to about 400. In one embodiment, the degree of polymerization is about 30 to about 400. In one embodiment, the degree of polymerization is about 40 to about 400. In one embodiment, the degree of polymerization is about 50 to about 400. In one embodiment, the degree of polymerization is about 60 to about 400. In one embodiment, the degree of polymerization is about 70 to about 400. In one embodiment, the degree of polymerization is about 80 to about 400. In one embodiment, the degree of polymerization is about 90 to about 400. In one embodiment, the degree of polymerization is about 100 to about 400. In one embodiment, the degree of polymerization is about 10 to about 300. In one embodiment, the degree of polymerization is about 20 to about 300. In one embodiment, the degree of polymerization is about 30 to about 300. In one embodiment, the degree of polymerization is about 40 to about 300. In one embodiment, the degree of polymerization is about 50 to about 300. In one embodiment, the degree of polymerization is about 60 to about 300. In one embodiment, the degree of polymerization is about 70 to about 300. In one embodiment, the degree of polymerization is about 80 to about 300. In one embodiment, the degree of polymerization is about 90 to about 300. In one embodiment, the degree of polymerization is about 100 to about 300. In one embodiment, the degree of polymerization is about 50 to about 250. In one embodiment, the degree of polymerization is about 60 to about 250. In one embodiment, the degree of polymerization is about 70 to about 250. In one embodiment, the degree of polymerization is about 80 to about 250. In one embodiment, the degree of polymerization is about 90 to about 250. In one embodiment, the degree of polymerization is about 100 to about 250.

In one embodiment, the polymerization is performed at about 25° C. to about 100° C. In one embodiment, the polymerization is performed at about 25° C. to about 90° C. In one embodiment, the polymerization is performed at about 25° C. to about 80° C. In one embodiment, the polymerization is performed at about 35° C. to about 80° C. In one embodiment, the polymerization is performed at about 45° C. to about 80° C. In one embodiment, the polymerization is performed at about 55° C. to about 80° C. In one embodiment, the polymerization is performed at about 60° C. to about 80° C. In one embodiment, the polymerization is performed at about 60° C. to about 90° C. In one embodiment, the polymerization is performed at about 60° C. to about 100° C.

In one embodiment, the ROMP catalyst comprises a transition metal. In one embodiment, the ROMP catalyst comprises a metal selected from the group consisting of titanium, molybedenum, tungsten, tantalum, rhenium, ruthenium, any oxidation state thereof, and any combination thereof. In one embodiment, the ROMP catalyst comprises a carbene. In one embodiment, the ROMP catalyst comprises a halide. In one embodiment, the ROMP catalyst comprises a phosphine ligand. In one embodiment, the ROMP catalyst is selected from the group consisting of first-generation Grubbs catalyst (Grubbs I), second-generation Grubbs catalyst (Grubbs II), and third-generation Grubbs catalyst (Grubbs III). In one embodiment, the ROMP catalyst is air stable. In one embodiment, the ROMP catalyst is stable to moisture.

In one embodiment, the step of polymerizing the monomer using a ROMP catalyst is performed in a glove box. In one embodiment, the step of polymerizing the monomer is done in an air-free environment. In one embodiment, the step of polymerizing the monomer is done open to air. In one embodiment, the step of polymerizing the monomer further comprises the step of adding the monomer and ROMP catalyst with at least one solvent to a solution. In one embodiment, the solvent is organic. Exemplary organic solvents include, but are not limited to, acetic acid, acetone, acetonitrile, alkanes (e.g., hexanes, heptane), amyl acetate, butanol, butyl acetate, chlorobenzene, chloroform, cumene, cyclohexane, 1,2-dichloroethene, dichloromethane, diethyl ether, dimethoxyethane, dimethylacetamide, dimethylformamide, dimethyl sulfoxide, 1,4-dioxane, ethanol, 2-ethoxyethanol, ethyl acetate, ethyl nitrate, ethyleneglycol, formic acid, hydrazine, isopropanol, methanol, methyl acetate, 2-methyl-1-butanol, 2-methyl-1-propanol, methylbutyl ketone, methylcyclohexane, methylethyl ketone, methylpyrrolidone, methyl tert-butyl ether, nitromethane, propanol, propyl acetate, sulfolane, sarcosine, tetrahydrofuran, tetralin, toluene, 1,1,2-tricholoroethane, triethylamine, urea, xylene, and any combination thereof. In one embodiment, the step of polymerizing the monomer is automated. In one embodiment, the step of polymerizing the monomer is performed on a stir plate. In one embodiment, the step of polymerizing the monomer is performed on a hot plate. In one embodiment, the step of polymerizing the monomer is performed using a heating block.

In one embodiment, the polymerization step is monitored by percent conversion. In one embodiment, “percent conversion” is defined as the percentage of monomer converted to polymer. In one embodiment, the polymerization reaches full conversion of monomer to polymer after 1 minute. In one embodiment, the polymerization reaches full conversion after about 1 minute to about 5 minutes. In one embodiment, the polymerization reaches full conversion after about 5 minutes to about 10 minutes. In one embodiment, the polymerization reaches full conversion after about 10 minutes to about 30 minutes. In one embodiment, the polymerization reaches full conversion after about 30 minutes to about 60 minutes. In one embodiment, the polymerization reaches full conversion after about 60 minutes.

In one embodiment, the polymerization is performed using two or more monomers comprising different carbohydrates. In one embodiment, the polymerization step is performed using a monomer which does not comprise a carbohydrate. In one embodiment, the polymerization step is performed on two or more monomers with a ROMP catalyst to produce a random copolymer. In one embodiment, the polymerization step is performed to produce a statistical copolymer. In one embodiment, the polymerization step is performed on two or more monomers to produce a block copolymer. In one embodiment, the polymerization step is performed on two or more monomers to produce an alternating copolymer. In one embodiment, the polymerization step is performed to produce a stereoblock copolymer. In one embodiment, the polymerization step is performed to produce a gradient copolymer.

In one embodiment, a method of polymerization alternative to ROMP is used to produce a copolymer of the carbohydrate-polymer conjugate. Exemplary methods of polymerization include, but are not limited to, atom transfer free radical polymerization (ATRP), reversible addition fragmentation chain transfer (RAFT) polymerization, anionic polymerization, cationic polymerization, living polymerization, chain shuttling polymerization, free radical polymerization, and nitroxide mediated radical polymerization (NMP).

In one embodiment, the carbohydrate-polymer conjugate is isolated by evaporating solvent. In one embodiment, the carbohydrate-polymer conjugate is isolated by filtration. In one embodiment, the carbohydrate-polymer conjugate is isolated by precipitation. In one embodiment, the carbohydrate-polymer conjugate is isolated using drying. In one embodiment, the carbohydrate-polymer conjugate is isolated using dialysis.

### Methods of Use

The present invention is further drawn to, in part, polymers and/or compositions which can be used to modulate the function of a cell (e.g., immune cell). Thus, in one embodiment, the present invention provides a method to induce an immune response. In one embodiment, the present invention provides a method to induce suppression of a cancer cell. In one embodiment, the present invention provides a method to induce cell death. In one embodiment, the present invention provides a method to reduce proliferation of a cancer cell. In one embodiment, the present invention provides a method to reduce cancer cell migration.

In one embodiment, the present invention provides a method comprising the step of administering to the subject a compound or composition comprising a carbohydrate-polymer conjugate comprising a structure represented by Formula (I):

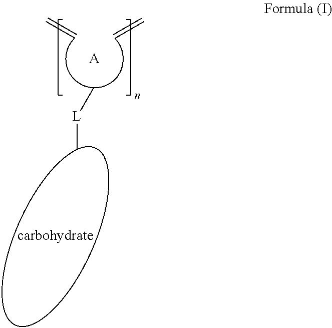

wherein: A represents a repeat unit selected from the group consisting of C₅-C₇ alkyl, C₅-C₇ heteroalkyl, C₅-C₇ aryl, C₅-C₇ heteroaryl, or C₅-C₇ cycloalkyl; n represents an integer from 10-500; L represents a divalent organic linker; and wherein L forms a direct bond to an anomeric position of the carbohydrate.

Thus, in various embodiments, the present invention provides a method of treating or preventing a disease or disorder associated with immune cell function, immune response, activation of a cell, activation of an immune cell, or any combination thereof in a subject in need thereof. In one embodiment, the present invention provides a method of treating or preventing a disease or disorder in a subject in need thereof. In one embodiment, the disease or disorder is a disease or disorder associated with abnormal immune cell function in a subject in need thereof.

In one embodiment, the present invention provides a method of treating or preventing a cancer in a subject in need thereof.

In one embodiment, the compound or composition binds to a protein which participates in the progression of a disease. In one embodiment, the compound or composition binds to a protein which binds carbohydrates. In one embodiment, the compound or composition binds to a protein which binds to β-galactosides. In one embodiment, the compound or composition binds to a protein which binds to glucosides. In one embodiment, the compound or composition binds to a protein which binds to mannosides. In one embodiment, the composition binds to a target which is involved in the progression of cancer. In one embodiment, the composition binds to a lectin. The term “lectin” as used herein refers to a carbohydrate-binding protein.

In one embodiment, the composition binds to a target protein selected from Gal-3, MGL, ASGPR, MBL, DC-SIGN, Langerin, GLUT-1, GLUT-2, GLUT-3, variants thereof, and combinations thereof. Examples of various cancers include but are not limited to, breast cancer, prostate cancer, ovarian cancer, cervical cancer, skin cancer, pancreatic cancer, colorectal cancer, renal cancer, liver cancer, brain cancer, head and neck cancers, lymphoma, leukemia, lung cancer and the like.

In one embodiment, the compound or composition inhibits proliferation of cancer cells. In one embodiment, the compound or composition inhibits migration of cancer cells. In one embodiment, the inhibition of proliferation and/or migration of cancer cells is dependent on the concentration of the compound or composition. In one embodiment, the compound or composition inhibits tumor growth. In one embodiment, the compound or composition inhibits at least one cancer cell from metastasizing.

Any therapeutic agent or any combination of therapeutic agents disclosed herein may be administered to a subject to treat a disease or disorder. The therapeutic agents herein can be formulated in any number of ways, often according to various known formulations in the art or as disclosed or referenced herein.

In certain embodiments, the method of treating a disease or disorder comprises a “triggered” functionality. In other words, the system may remain inert in the body until specifically triggered. In some embodiments, the polymer is used advantageously in therapeutic applications such as to first target the polymer to a specified location, and then trigger them into an activated state. Sometimes referred to as a “dual targeted delivery system,” this feature may minimize the side effects of systemic therapeutic agents. For example, in some embodiments, upon delivering the polymer to a specific cell, a reagent, such as water, proton, acid, or protonated water, may be applied to the cell thereby causing the release of a therapeutic agent from the polymer. In some embodiments, this may provide a clinician the ability to control and visualize drug therapy noninvasively.

In some embodiments, the size (e.g., average diameter of a polymer assembly) of the compound or composition of the present invention allows for passive diffusion into cells. In some embodiments, where the compound or composition is on a smaller scale, the small size (e.g., average diameter of a polymer assembly) allows the compound or compositions to travel almost anywhere in the body where therapy may need to be performed. For example, in some embodiments, the method comprises compounds that act as a hydrolysis triggered therapeutic agent delivery and therapeutic agent release systems.

In one embodiment, the compound or composition undergoes uptake into cells. In one embodiment, the compound or composition undergoes uptake into cancer cells. In one embodiment, the compound or composition undergoes uptake into breast cancer cells. In one embodiment, the compound or composition undergoes uptake into liver cancer cells. In one embodiment, the compound or composition undergoes uptake into macrophage cells. In one embodiment, the compound or composition undergoes uptake into dendritic cells. For example, in one embodiment, the compound or composition can be coated with dextran to target the macrophage cells, since macrophages have dextran receptors. In various embodiments, the method further comprises allowing the compound or composition to accumulate in a region of the biological tissue, wherein the targeting domain facilitated accumulation of the compound, nanoparticle, or composition in the region. In one embodiment, the compound or composition localizes around the exterior of the nucleus.

In various aspects, the compound or composition of the present invention can be used alone or in combination with a therapeutic agent to deliver a therapeutic agent payload to a target cell. Often, the therapeutic agent may be released based on the degradation of, e.g., a controlled release biodegradable matrix and/or polymer.

The preferred dosage of the compound or nanoparticle will vary according to a number of factors, such as the administration route, the age, weight and species of the subject, but in general containing in the order of from 1 μmol/kg to 1 mmol/kg bodyweight of the compound or nanoparticle.

Administration may be topical, parenteral (e.g., intravenously, intraarterially, intramuscularly, interstitially, subcutaneously, transdermally, or intrasternally), or into an externally voiding body cavity (e.g., the gastrointestinal tract, rectum, bladder, uterus, vagina, nose, ears or lungs), peritoneally, orally, intradermal, ocular, in an animate human or non-human (e.g., mammalian, reptilian or avian) body.

In certain embodiments, the compound or composition herein is used in conjunction with an anti-cancer agent known in the art. Exemplary anti-cancer agents include, but are not limited to, immunotherapy agents, immunomodulatory agents, antineoplastic agents, chemotherapeutic agents, radioimmunotherapy agents, and monoclonal antibodies.

In some embodiments, the anti-cancer agent may be a prodrug form of an anti-cancer agent. As used herein, the term “prodrug form” and its derivatives is used to refer to a drug that has been chemically modified to add and/or remove one or more substituents in such a manner that, upon introduction of the prodrug form into a subject, such a modification may be reversed by naturally occurring processes, thus reproducing the drug. The use of a prodrug form of an anti-cancer agent in the compositions, among other things, may increase the concentration of the anti-cancer agent in the compositions of the present disclosure. In certain embodiments, an anti-cancer agent may be chemically modified with an alkyl or acyl group or some form of lipid. The selection of such a chemical modification, including the substituent(s) to add and/or remove to create the prodrug, may depend upon a number of factors including, but not limited to, the particular drug and the desired properties of the prodrug. One of ordinary skill in the art, with the benefit of this disclosure, will recognize suitable chemical modifications.

### Embodiments

1. A carbohydrate-polymer conjugate comprising a structure represented by Formula (I):

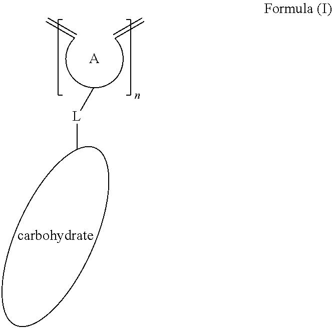

wherein:
A represents a repeat unit selected from the group consisting of C₅-C₇ alkyl, C₅-C₇ heteroalkyl, C₅-C₇ aryl, C₅-C₇ heteroaryl, or C₅-C₇ cycloalkyl;
n represents an integer from 10-500;
L represents a divalent organic linker; and
wherein L forms a direct bond to an anomeric position of the carbohydrate.
2. The carbohydrate-polymer conjugate of embodiment 1, wherein ring A is represented by one of the following structures:

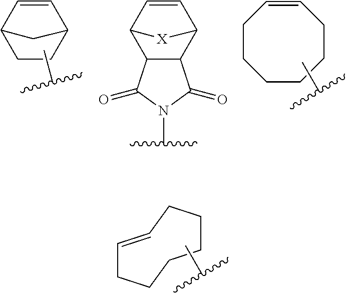

wherein the wave line represents a bond to L;
X represents O, S, NR¹, or CR²R³;
R¹, R², and R³ each independently represent a substituent selected from the group consisting of hydrogen, halide, hydroxy, C₁-C₃₀ alkyl, C₁-C₃₀ alkyl halide, C₁-C₃₀ alkoxy, and any combination thereof, and
each structure is independently optionally further substituted.
3. The carbohydrate-polymer conjugate of embodiment 1 or 2, wherein the polymer backbone is unsaturated.
4. The carbohydrate-polymer conjugate of any one of embodiments 1-3, wherein L is represented by one of the following structures:

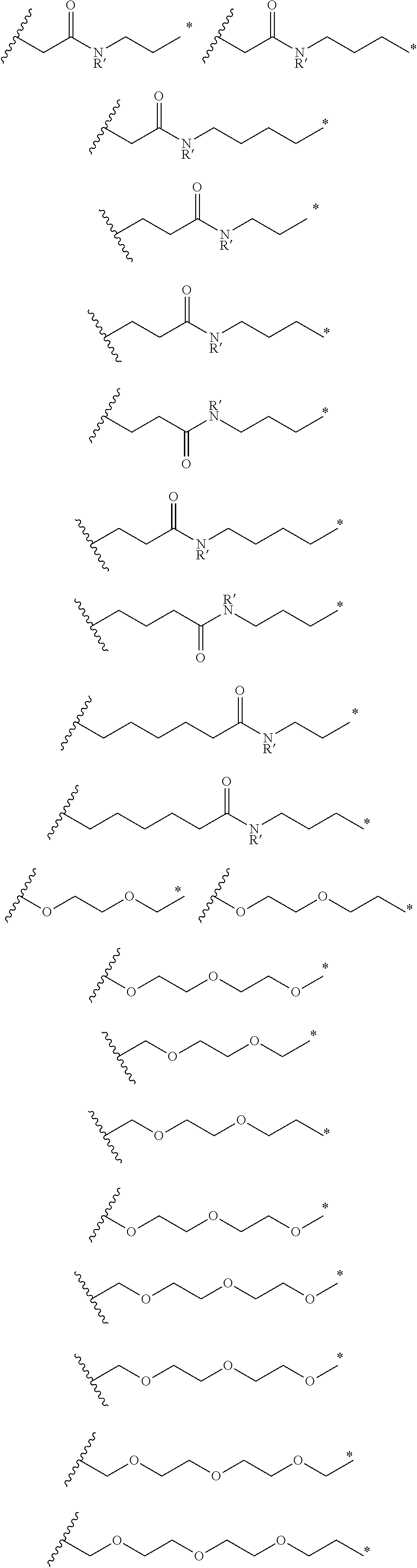

wherein the wavy line represents a bond to ring A;
* represents a bond to the carbohydrate; and
R′ represents a substituent selected from the group consisting of hydrogen, halide, hydroxy, C₁-C₃₀ alkyl, C₁-C₃₀ alkyl halide, C₁-C₃₀ alkoxy, and any combination thereof.
5. The carbohydrate-polymer conjugate of anyone of embodiments 1-4, wherein L comprises a C₁-C₃₀ alkyl which is optionally further substituted.
6. The carbohydrate-polymer conjugate of anyone of embodiments 1-5, wherein the carbohydrate is selected from the group consisting of a monosaccharide, a disaccharide, an oligosaccharide, a polysaccharide, any stereochemical isomer thereof, and any combination thereof.
7. The carbohydrate-polymer conjugate of anyone of embodiments 1-6, wherein the carbohydrate is selected from the group consisting of galactose, glucose, mannose, allose, altrose, gulose, idose, talose, psicose, fructose, sorbose, tagatose, and any stereochemical isomer thereof.
8. The carbohydrate-polymer conjugate of anyone of embodiments 1-7, wherein the carbohydrate-polymer conjugate is a copolymer comprising a repeat unit of at least two monomers, wherein at least one monomer comprises galactose.
9. The carbohydrate-polymer conjugate of anyone of embodiments 1-8, wherein the carbohydrate is acetylated.
10. A composition comprising the carbohydrate-polymer conjugate of anyone of embodiments 1-9.
11. A method of synthesizing a carbohydrate-polymer conjugate using ring-opening metathesis polymerization (ROMP), comprising the steps of:
   providing a monomer;
   polymerizing the monomer using a ROMP catalyst; and
   isolating the carbohydrate-polymer conjugate;
   wherein the monomer is represented by Formula (I):

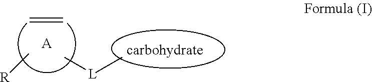

wherein:
R represents a substituent on ring A selected from the group consisting of hydrogen, halide, hydroxy, C₁-C₃₀ alkyl, C₁-C₃₀ alkyl halide, C₁-C₃₀ alkoxy, and any combination thereof, and
L represents a divalent organic linker.
12. The method of embodiment 11, wherein the degree of polymerization is about 10 to about 500.
13. The method of embodiment 11 or 12, wherein the polymerization is performed at about 60° C.
14. A method comprising the step of administering to the subject a composition comprising a carbohydrate-polymer conjugate comprising a structure represented by Formula (I):

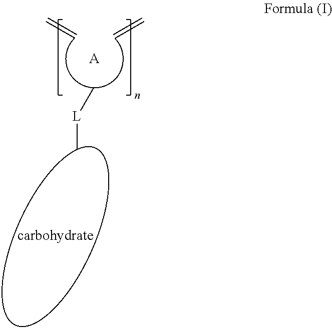

wherein:
A represents a repeat unit selected from the group consisting of C₅-C₇ alkyl, C₅-C₇ heteroalkyl, C₅-C₇ aryl, C₅-C₇ heteroaryl, or C₅-C₇ cycloalkyl;
n represents an integer from 10-500;
L represents a divalent organic linker; and
wherein L forms a direct bond to an anomeric position of the carbohydrate.
15. The method of embodiment 14, wherein the subject has cancer.
16. The method of embodiment 15, wherein the composition suppresses proliferation of a cancer cell.
17. The method of embodiment 15 or 16, wherein the composition inhibits tumor growth.
18. The method of any one of embodiments 15-17, wherein the composition inhibits at least one cancer cell from metastasizing.
19. The method of any one of embodiments 14-18, wherein the composition binds to a lectin.
20. The method of any one of embodiments 14-19, wherein the composition binds to a target protein selected from Gal-3, MGL, ASGPR, MBL, DC-SIGN, Langerin, GLUT-1, GLUT-2, GLUT-3, variants thereof, and combinations thereof.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Elucidating the Relationship Between Glycan Macromolecular Structure, Recognition, and Anticancer Function

The present invention is drawn to, in part, carbohydrate-polymer conjugates (CPCs) with unambiguous local chemical composition and well-defined global structure for use as tools to understand the nature of multivalent carbohydrate recognition. The mechanisms governing how the hierarchical arrangement of glycans potentiates their recognition by human lectins remain incomplete. Initial investigations were performed on the binding of β-galactosides to Galectin-3 (Gal-3), a protein with vital roles in myriad diseases, including cancer. β-galactose and lactose were derivatized with norbornenyl moieties to afford protecting-group-free glycomonomers. Using direct, graft-through ring opening metathesis polymerization (ROMP), a suite of CPCs was generated and systematically evaluated their ability to bind to Gal-3. Significantly, galactose-based CPCs (Gal-CPCs) showed a ca. 3600-fold improvement in Gal-3 binding over the monosaccharide ligand. Moreover, complete grafting density was required for maximal binding to Gal-3, with Gal-CPCs displaying more than a 350% enhancement in binding to Gal-3 over copolymers at 50% galactose density. Coarse-grain models suggest that this arises from reduced carbohydrate-carbohydrate interactions and enhanced solvent accessibility in fully grafted CPCs. This translates to significant differences in the function of these materials as Gal-3 inhibitors in in vitro models of 4T1 triple-negative breast cancer. Gal-CPCs inhibited the migration of 4T1 cells by greater than 60% relative to vehicle controls and significantly outperformed all other treatment groups. Together, these findings shed light on how the macromolecular structure of carbohydrates dictates their ability to bind human lectins and paves the way to the development of polymeric cancer therapeutics comprised of simple, inexpensive β-galactosides.

To elucidate the mechanisms underlying carbohydrate recognition by mammalian lectins, glycopolymer probes were developed with precise local chemical composition and well-defined global macromolecular structure. It was reasoned that the graft-through ring opening metathesis polymerization (ROMP) of unprotected carbohydrates would afford well-defined glycopolymers with maximal grafting density and unambiguous local chemical structure (i.e., each sidechain is functionalized, and no post-polymerization manipulations are required). ROMP is ideal in this regard, as it is a controlled polymerization method (Bandlow et al., 2017, J. Am. Chem. Soc., 139, 16389) that allows for the generation of polymers with low dispersity and high reproducibility. Its initiators (Xiong et al., 2013, Nature, 497, 392; display excellent functional group tolerance, which permits the incorporation of a diverse array of functionalized monomers, such as the dense concentration of hydroxyl moieties on carbohydrates (Kanai et al., 1997, J. Am. Chem. Soc., 119, 9931; Mortell et al., 1994, J. Am. Chem. Soc., 116, 12053; Tang et al., 2016, ACS Macro Lett., 5, 413; Fraser et al., 1995, Macromolecules, 28, 7248). Moreover, the rigid, sp²-hybridized backbones of poly(norbornene) are akin to the polypeptide backbones on which oligosaccharides are natively displayed.

As a model system, the binding of β-galactosides by galectin-3 (Gal-3), a protein involved in myriad physiological processes (Joeh et al., 2020, Proc. Natl. Acad. Sci., 117, 27329) and in the progression of multiple diseases (Califice et al., 2004, Int. J. Oncol., 25, 983; Feilchenfeldt et al., 2003, Modern Pathology, 16, 1117; Gao et al., 2013, Respiratory Research, 14, 136), including cancer, was studied. As such, understanding the nature of its carbohydrate recognition would have wide-ranging biomedical implications (Farhadi et al., 2021, Proc. Natl. Acad. Sci. USA, 118; Pradmudya, 2019, Biomaterials Science, 7, 4848). Like other galectin family members, Gal-3 recognizes and binds β-galactosides in a multivalent fashion (Restuccia et al., 2018, ACS Biomat. Sci. Eng., 4, 3451; Laaf et al., 2017, Bioconjugate Chemistry, 28, 2832)). Gal-3 is overexpressed in many cancers, where it inhibits immune cell action in the tumor microenvironment (Guo et al., 2020, Oncol. Rep., 44, 1799), contributes to cell proliferation (Paron et al., 2003, Biochem. Biophys. Res. Commun., 302, 545; Preillo et al., 1998, J. Mol. Med., 76, 402), and promotes cancer cell migration (Liu, 2005, Nat. Rev. Cancer, 5, 29). Previous research on targeting Gal-3 has focused on synthetically laborious small molecule inhibitors (Giguere et al., 2006, Chem. Comm., 22, 2379; Zetterberg et al., 2022, J. Med. Chem., 65, 12626). Of note is TD139, a small molecule thiodigalactoside against Gal-3 is currently undergoing clinical trials for idiopathic pulmonary fibrosis (Nikhil et al., 2021, Eur. Resp. J., 57, 2002559). While there are two reports on polymers designed to bind to Gal-3, neither have achieved more than a ~16% grafting density (Filipova et al., 2020, Biomacromolecules, 21, 3122; Vrbata et al., 2022, J. Med. Chem., 65, 3866) and utilize post-polymerization modification methods. Moreover, these reports focus on therapeutic development, with little insight on a fundamental understanding of the interactions between Gal-3 and multivalent ligands.

Given these facts, it was reasoned that understanding the macromolecular nature of Gal-3 binding to β-galactosides could both offer fundamental insights into carbohydrate recognition and provide a potential pathway to the development of new polymeric materials for cancer therapy. Herein, carbohydrate-polymer conjugates (CPCs) have been synthesized via the direct, graft-through polymerization of two β-galactoside ligands for Gal-3. These materials were used to evaluate how carbohydrate multivalency and density influences lectin recognition and therapeutic function. It was found that the multivalent display of carbohydrates overrides differences in both individual ligand affinities and overall degree of polymerization. Moreover, complete grafting density is required for maximal binding to Gal-3. Coarse-grain analyses offer mechanistic insight into the nature of these interactions, indicating that fully grafted CPCs outperformed others due to their highest solvent-accessible surface area and reduced carbohydrate-carbohydrate interactions. Significantly, these differences translate into distinct actions of CPCs as galectin-3 inhibitors in in vitro models of triple-negative breast cancer. Fully grafted CPCs inhibit the migration of 4T1 cells by over 60% compared to vehicle controls. Together, these findings provide insight on how the macromolecular density of carbohydrates modulates their recognition by proteins and can guide the design of multivalent galectin-targeting materials for applications in cancer and beyond.

### Glycomonomer Design

To probe the effect of multivalency on the binding of β-galactosides to Gal-3, β-galactose and lactose were chosen as model ligands as they are the simplest naturally occurring glycans recognized by Gal-3 (Nio-Kobayashi, 2021, Front. Neuroanat., 15) and have vastly different affinities for the lectin (~150 mM for galactose, 200 μM for lactose) (Saraboji et al., 2012, Biochemistry, 51, 296). Moreover, previous reports have found that longer linkers on polymer sidechains increase self-aggregation from intramolecular hydrophobic and hydrogen bonding (Mosquera-Giraldo et al., 2016, Biomacromol., 17, 3659). Therefore, it was hypothesized that β-galactosides bound by a shorter linker would exhibit improved binding potency over those with longer linkers. Thus, both galactose and lactose were derivatized with norbornenyl handles with variations in the spacing between carbohydrate ligand and polymerizable moiety.

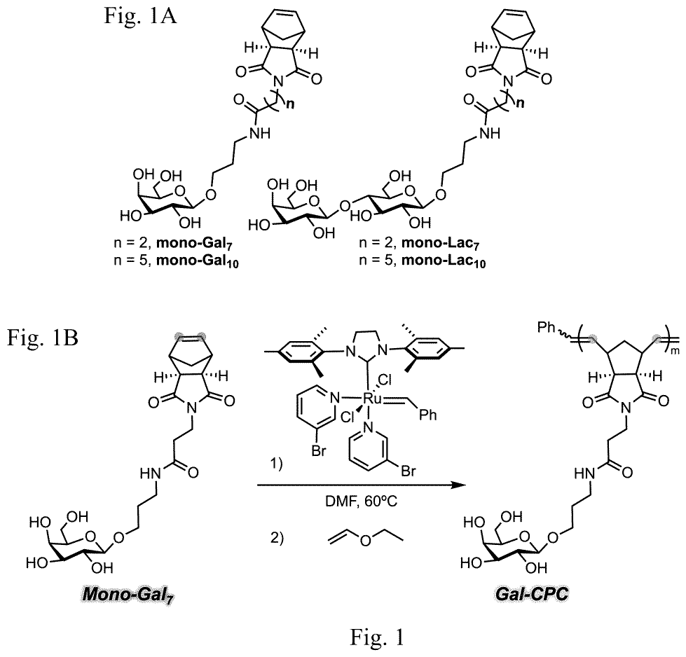

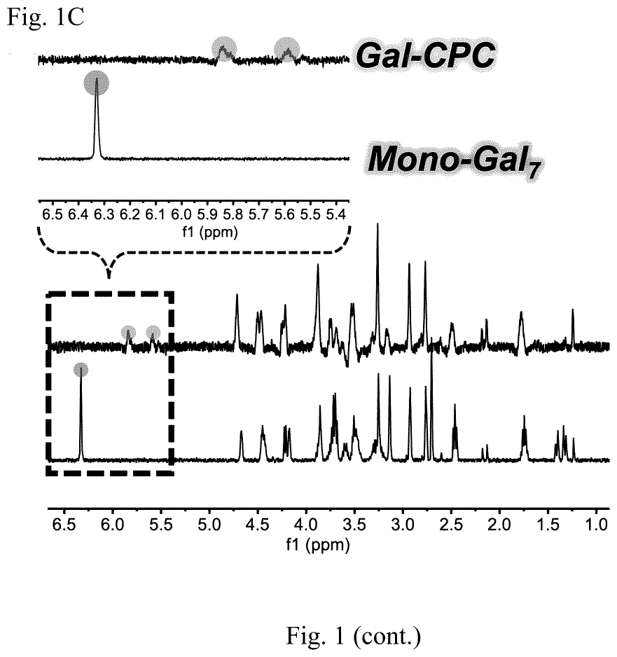

With these design considerations in mind, two glycomonomers were synthesized from galactose (mono-Gal₇ and mono-Gal₁₀) and two glycomonomers were synthesized from lactose (mono-Lac₇ and mono-Lac₁₀) with 7 and 10 atoms between glycan epitope and norbornenyl handle, respectively (FIG. 1A). Briefly, a norbornenyl moiety was appended with an amino acid, followed by addition of an amino alcohol using standard amidation conditions. The linker was formed using two components so that an amide could be incorporated, as it was hypothesized that a purely hydrocarbon-based linker may overwhelm the hydrophilicity of the carbohydrate and reduce solubility. Moreover, an amide linker provides hydrogen-bonding capability and a way to rapidly change the linker length. Lastly, the norbornenyl-linker conjugate was conjugated with a commercially available acetylated sugar to generate a protected glycomonomer that, following deprotection, yields the final glycomonomer with a native glycosidic bond, which was hypothesized to play a role in the recognition of the carbohydrate by Gal-3.

### Carbohydrate-Polymer Conjugate (CPC) Synthesis and Analysis.

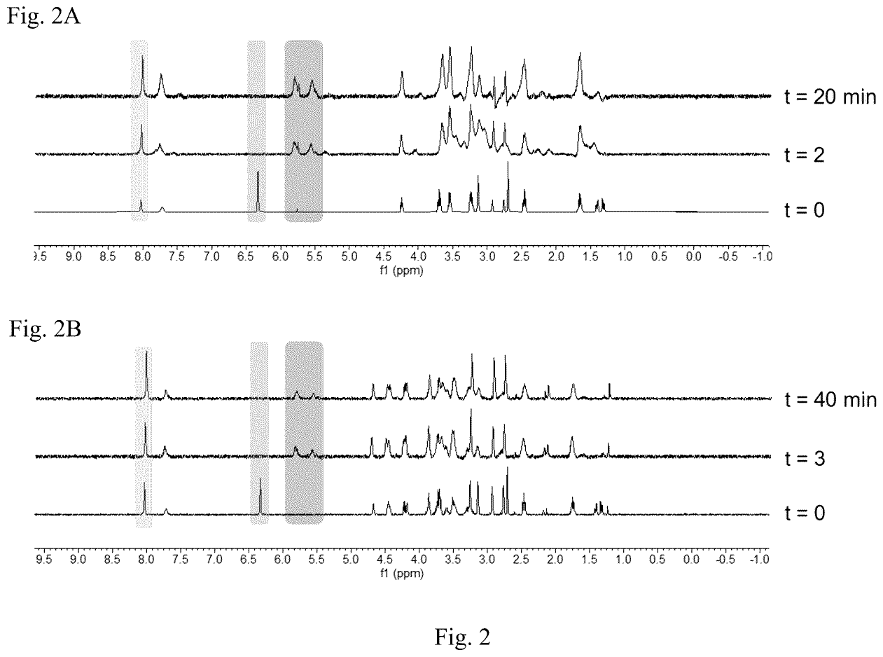

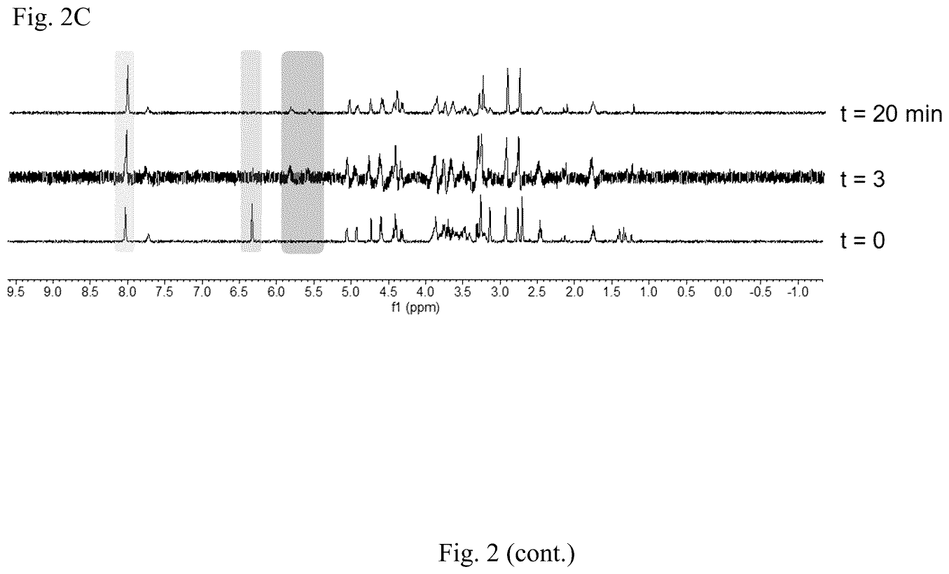

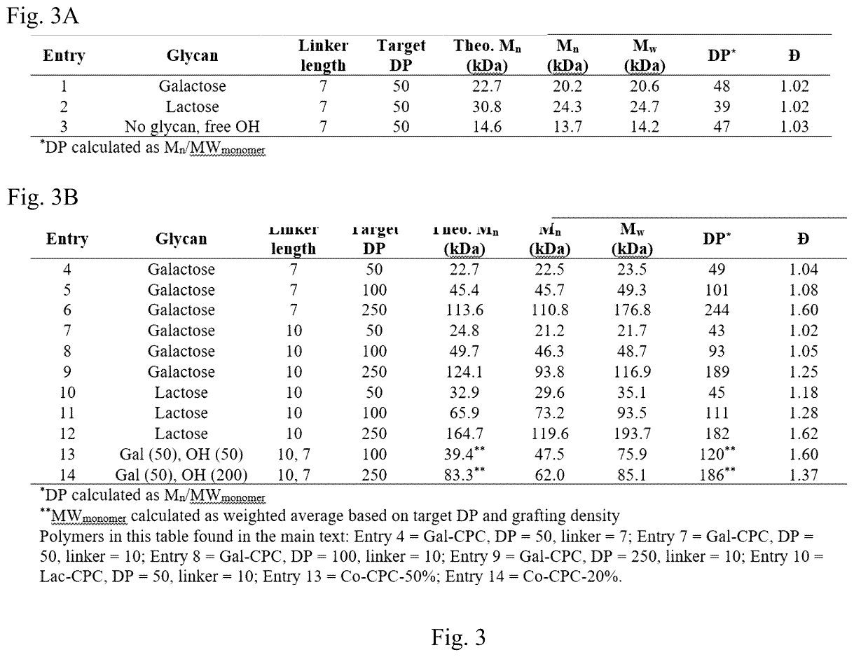

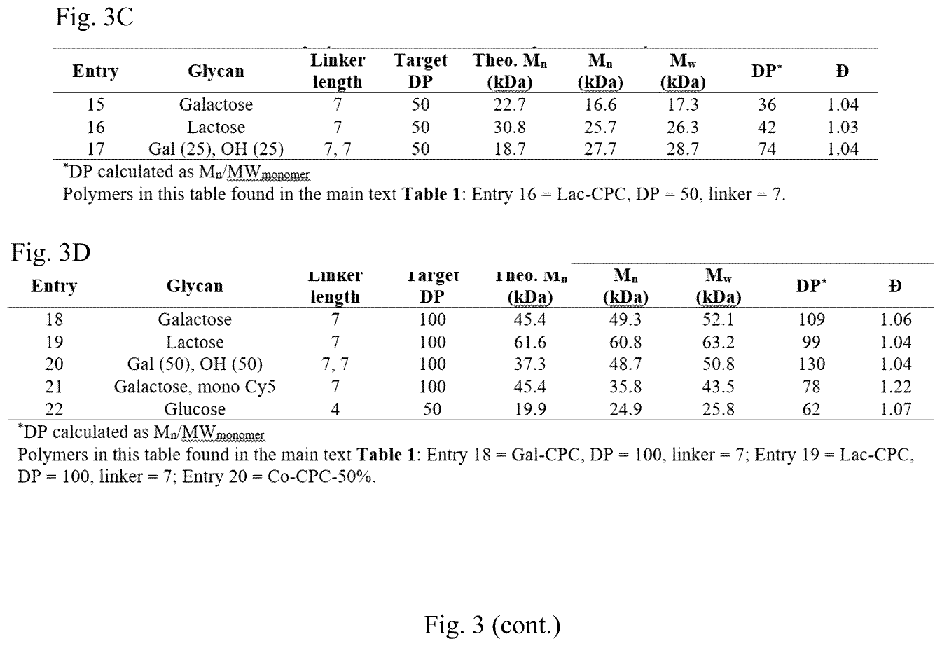

With glycomonomers in hand, each was subjected to ROMP using Grubb's III generation catalyst in dry DMF at 60° C. to afford CPCs (FIG. 1). Altogether, a suite of 12 total CPCs from galactose (Gal-CPC) and lactose (Lac-CPC) with variations in linker length (7 or 10 atoms) and target degree of polymerization (50, 100, or 250) was synthesized. An attempt was made to monitor the polymerization progress of mono-Gal₇ by ¹H-NMR, but complete conversion occurred in less than 5 minutes (FIG. 1C). Likewise, all other glycomonomers were also efficiently converted to CPCs within the same timeframe (FIG. 2). Following polymerization, all CPCs were characterized using size exclusion chromatography with multi-angle light scattering (SEC-MALS) to determine the degree of polymerization (DP) and dispersity (Ð) (Table 1, FIG. 3).

TABLE 1
Polymer Characterization by SEC-MALS.

| | | Linker | Target | Theo. | Mₙ | M_{w} | | |
|---|---|---|---|---|---|---|---|---|
| Entry | Polymer | Length | DP | Mₙ (kDa) | (kDa) | (kDa) | DP^{*} | Ð |
| 1 | Gal-CPC, DP = 50 | 7 | 50 | 22.7 | 22.5 | 23.5 | 49 | 1.043 |
| 2 | Gal-CPC, DP = 100 | 7 | 100 | 45.4 | 48.1 | 50.3 | 106 | 1.045 |
| 3 | Gal-CPC, DP = 250 | 7 | 250 | 113.6 | 94.1 | 107.9 | 207 | 1.147 |
| 4 | Gal-CPC, DP = 50 | 10 | 50 | 24.8 | 21.2 | 21.7 | 43 | 1.023 |
| S | Gal-CPC, DP =100 | 10 | 100 | 49.7 | 46.3 | 48.7 | 93 | 1.053 |
| 6 | Gal-CPC, DP = 250 | 10 | 250 | 124.1 | 93.8 | 116.9 | 189 | 1.247 |
| 7 | Lac-CPC, DP = 50 | 7 | 50 | 30.8 | 26.9 | 27.5 | 44 | 1.018 |
| 8 | Lac-CPC, DP = 100 | 7 | 100 | 61.7 | 60.8 | 63.2 | 99 | 1.04 |
| 9 | Lac-CPC, DP = 250 | 7 | 250 | 154.2 | 129.9 | 138.7 | 211 | 1.068 |
| 10 | Lac-CPC, DP = 50 | 10 | 50 | 32.9 | 29.6 | 35.1 | 45 | 1.184 |
| | Lac-CPC, DP =100 | 10 | 100 | 65.9 | 57.4 | 59.5 | 87 | 1.038 |
| 12 | Lac-CPC, DP = 250 | 10 | 250 | 164.7 | 156.8 | 174 | 238 | 1.110 |

^{*}DP calculated as Mₙ/MWₘₒₙₒₘₑᵣ

The measured degree of polymerization (DP) closely matched the theoretical DP calculated from the initial monomer-to-initiator ratio ([M]₀/[I]₀) for each polymer, and all D were below 1.25. This suggests a controlled polymerization process with narrow molecular weight distributions and afforded well-defined CPCs for use in future analyses.

### Galectin-3 Binding as a Function of Valency, Linker, and Glycan Identity.

The ability of all CPCs to be recognized by Galectin-3 were investigated in vitro. For these analyses, microscale thermophoresis (MST) was utilized, as it is a fully solution-based technique that measures the motion of fluorescent molecules in response to a microscopic temperature gradient (Bandlow et al., 2017, J. Am. Chem. Soc., 139, 16389; Xiong et al., 2013, Nature, 497, 392). It was posited that this technique would more accurately recapitulate the natural interactions between the lectin and glycopolymer than would be obtainable by immobilization-based techniques (e.g., surface plasmon resonance). With this in mind, the fluorophore Atto 488 was conjugated to Gal-3 at its C-terminal domain so that the movement of the protein could be tracked without interfering with its β-galactoside binding site (Sorme et al., 2005, J. Am. Chem. Soc., 127, 1737). The suite of CPCs was then incubated with the tagged protein at increasing polymer concentrations and analyzed their interactions to determine their dissociation constants (K_{d}) as a function of β-galactoside identity, linker length, and overall degree of polymerization (Table 2).

TABLE 2
MST Analysis of Galectin-3 Binding by CPCs.

| Polymer | Linker Length | DP | k_{d} (μM)* |
|---|---|---|---|
| Gal-CPC | 7 | 50 | 81.1 ± 7.8 |
| | 7 | 100 | 42.1 ± 2.6 |
| | 7 | 250 | 44.7 ± 2.7 |
| | 10 | 50 | 88.4 ± 1.3 |
| | 10 | 100 | 45.6 ± 4.2 |
| | 10 | 250 | 55.8 ± 9.3 |
| Lac-CPC | 10 | 50 | 36.3 ± 2.8 |
| | 10 | 100 | 33.2 ± 4.5 |
| | 10 | 250 | 21.2 ± 1.7 |

*Error calculated as ±1 standard deviation (SD)

Through these analyses, it was discovered that displaying β-galactose on a polymer scaffold (i.e., Gal-CPCs) provides multivalent materials capable of binding Gal-3 with an apparent K_{d} in the mid-micromolar range. The most effective CPC exhibited approximately a 3600-fold improvement compared to monomeric β-galactose. This finding is consistent with observations in other glycomacromolecule systems (Cervin et al., 2020, ACS Infect. Dis., 6, 1192; Gonnot et al., 2023, Biomacromolecules, 24, 3689), where multivalency enhances potency in a non-linear manner, a phenomenon termed “avidity” (Zumbro et al., 2019, Biophys. J., 117, 892). Interestingly, there is a diminishing return on avidity as the degree of polymerization (DP) is increased. Gal-CPCs at DP=100 show approximately double the binding avidity over Gal-CPCs at DP=50; however, Gal-CPCs at DP=250 show no appreciable increase in avidity relative to DP=100. Moreover, the number of atoms linking the glycan epitope to the polymer backbone (i.e., 7 vs. 10 atoms) showed no effect on K_{d}.

When comparing Gal-CPCs to Lac-CPCs, unintuitive binding behavior was observed. Lac-CPCs were able to bind Gal-3 with higher avidity than Gal-CPCs at all DPs evaluated; however, the increase was marginal. This was unexpected, considering the vast differences in individual ligand affinities between galactose (~150 mM) and lactose (200 μM). Thus, it was concluded that the recognition of β-galactosides in this system is predominantly influenced by multivalency, rather than the specific identity of individual ligands. This observation may elucidate why Gal-3 exhibits the ability to bind various multivalent galactosides found in nature and why the most specific natural Gal-3 ligand remains unknown.

### Galectin-3 Binding as a Function of Glycan Epitope Density.

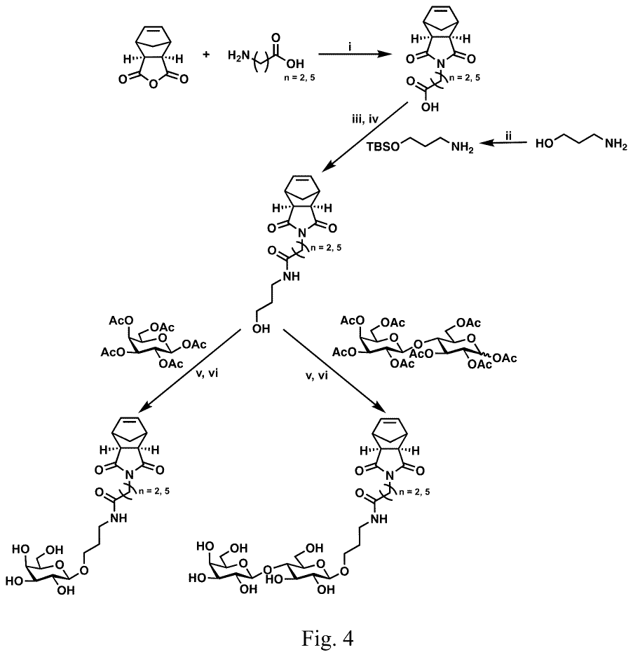

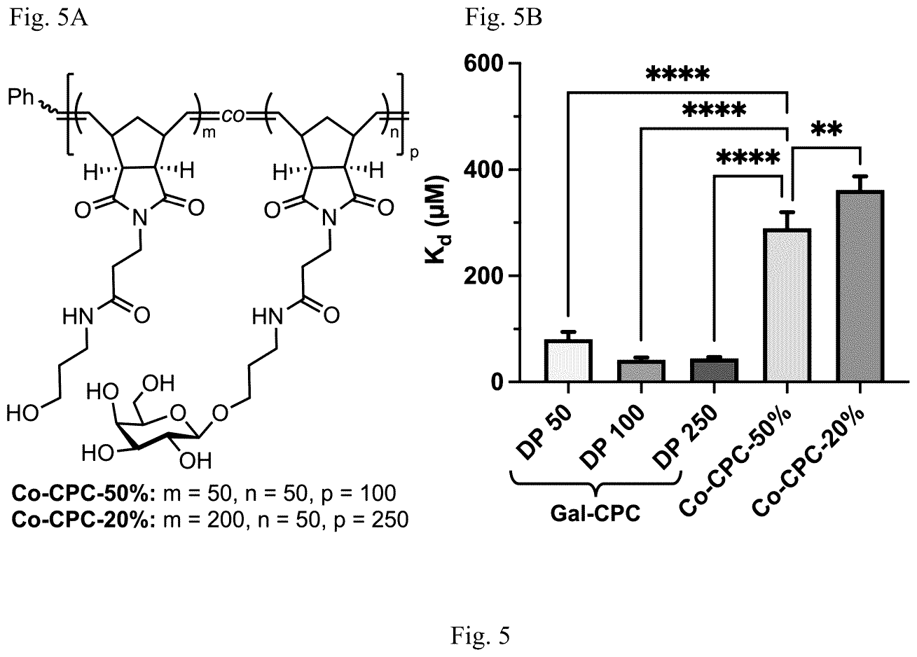

Motivated by the findings, the effect of multivalency on glycan recognition by Gal-3 was further investigated. Specifically, the impact of sugar epitope density on binding avidity was studied, considering previous findings indicating the significance of both density and valency in lectin binding (Qin et al., 2016, Biomaterials, 101, 189). Towards this end, a “diluent” monomer was synthesized, which contained a single hydroxyl moiety (mono-OH₇) in place of the glycan epitope (FIG. 4). Copolymers were synthesized together with mono-Gal₇ using ROMP at 1:1 and 5:1 ratios of diluent:galactose, respectively, to mimic a graft-to polymer at 50% and 20% grafting density (FIG. 5A, Table 3, Table 4).

TABLE 3
SEC-MALS data for polymers used in MST experiments.

| | | | | Theo. | | | | |
|---|---|---|---|---|---|---|---|---|
| | | Linker | Target | Mₙ | Mₙ | M_{w} | | |
| Entry | Glycan | length | DP | (kDa) | (kDa) | (kDa) | DP^{*} | Ð |
| 4 | Galactose | 7 | 50 | 22.7 | 22.5 | 23.5 | 49 | 1.04 |
| 5 | Galactose | 7 | 100 | 45.4 | 45.7 | 49.3 | 101 | 1.08 |
| 6 | Galactose | 7 | 250 | 113.6 | 110.8 | 176.8 | 244 | 1.60 |
| 7 | Galactose | 10 | 50 | 24.8 | 21.2 | 21.7 | 43 | 1.02 |
| 8 | Galactose | 10 | 100 | 49.7 | 46.3 | 48.7 | 93 | 1.05 |
| 9 | Galactose | 10 | 250 | 124.1 | 93.8 | 116.9 | 189 | 1.25 |
| 10 | Lactose | 10 | 50 | 32.9 | 29.6 | 35.1 | 45 | 1.18 |
| 11 | Lactose | 10 | 100 | 65.9 | 73.2 | 93.5 | 111 | 1.28 |
| 12 | Lactose | 10 | 250 | 164.7 | 119.6 | 193.7 | 182 | 1.62 |
| 13 | Gal (50), | 10, 7 | 100 | 39.4** | 47.5 | 75.9 | 120** | 1.60 |
| | OH (50) | | | | | | | |
| 14 | Gal (50), | 10, 7 | 250 | 83.3** | 62.0 | 85.1 | 186** | 1.37 |
| | OH (200) | | | | | | | |

^{*}DP calculated as Mₙ/MWₘₒₙₒₘₑᵣ
^{**}MWₘₒₙₒₘₑᵣ calculated as weighted average based on target DP and grafting density
Polymers in this table: Entry 4 = Gal-CPC, DP = 50, linker = 7; Entry 7 = Gal-CPC, DP = 50, linker = 10; Entry 8 = Gal-CPC, DP = 100, linker = 10; Entry 9 = Gal-CPC, DP = 250, linker = 10; Entry 10 = Lac-CPC, DP = 50, linker = 10; Entry 13 = Co-CPC-50%; Entry 14 = Co-CPC-20%.

TABLE 4
SEC-MALS data for polymers used in biological experiments.

| | | | | Theo. | | | | |
|---|---|---|---|---|---|---|---|---|
| | | Linker | Target | Mₙ | Mₙ | M_{w} | | |
| Entry | Glycan | length | DP | (kDa) | (kDa) | (kDa) | DP^{*} | Ð |
| 18 | Galactose | 7 | 100 | 45.4 | 49.3 | 52.1 | 109 | 1.06 |
| 19 | Lactose | 7 | 100 | 61.6 | 60.8 | 63.2 | 99 | 1.04 |
| 20 | Gal (50), | 7, 7 | 100 | 37.3 | 48.7 | 50.8 | 130 | 1.04 |
| | OH (50) | | | | | | | |
| 21 | Galactose, | 7 | 100 | 45.4 | 35.8 | 43.5 | 78 | 1.22 |
| | mono Cy5 | | | | | | | |
| 22 | Glucose | 4 | 50 | 19.9 | 24.9 | 25.8 | 62 | 1.07 |

^{*}DP calculated as Mₙ/MWₘₒₙₒₘₑᵣ
Polymers in this table: Entry 18 = Gal-CPC, DP = 100, linker = 7; Entry 19 = Lac-CPC, DP = 100, linker = 7; Entry 20 = Co-CPC-50%.

For these analyses, the theoretical DP of mono-Gal was held at 50 and the mol ratio of mono-OH₇ was varied to achieve the desired glycan composition. This yielded two copolymers: one copolymer with 50% galactose grafting density at DP=100 (Co-CPC-50%) and one copolymer with 20% galactose grafting density at DP=250 (Co-CPC-20%).

Strikingly, the ability of the copolymers to bind Gal-3 was significantly abrogated as compared to that of Gal-CPC (FIG. 5B, Table 5). Reducing the overall grafting density by 50% (Co-CPC-50%) reduced the binding avidity by ca. 350% relative to Gal-CPC at DP=50, when glycan epitope number is matched. When the overall degree of polymerization is kept constant, this discrepancy becomes even more pronounced. Gal-CPCs at DP=100 show an approximate 7-fold higher binding avidity to Gal-3 over Co-CPC-50%, and Gal-CPCs at DP=250 show an approximate 8-fold enhancement over Co-CPC-20%. This is counter to what has been observed in other glycomacromolecule systems (Kruger et al., 2021, ACS Cent. Sci., 7, 624; Matsuoka et al., 2021, Bioorg. Med. Chem. Lett., 52, 128389; Richards et al., 2012, Angew. Chem. Int. Ed., 51, 7812; Schuster et al., 1997, J. Mol. Catal. A: Chem., 116, 209; Wilkins et al., 2018, ACS Macro Lett., 7, 1498). As such, the solution behavior of these materials was investigated using computational models.

TABLE 5
Dissociation constants (K_{d}) for Gal-CPCs and copolymers.

| Polymer | Linker Length | DP | k_{d} (μM)* |
|---|---|---|---|
| Gal-CPC | 10 | 50 | 88.4 ± 1.3 |
| | 10 | 100 | 45.6 ± 4.2 |
| | 10 | 250 | 55.8 ± 9.3 |
| Co-CPC-50% | 10, 7** | 100 | 289.3 ± 17.5 |
| Co-CPC-20% | 10, 7** | 250 | 362.0 ± 14.7 |

*Error calculated as ±1 standard deviation (SD)
**mono-Gal; mono-OH

Reducing the overall grafting density by 50% (Co-CPC-50%) reduced the binding avidity by ca. 350% relative to Gal-CPC at DP=50, when glycan epitope number is matched. When the overall degree of polymerization is kept constant, this discrepancy becomes even more pronounced. Gal-CPCs at DP=100 show an approximate 7-fold higher binding avidity to Gal-3 over Co-CPC-50%, and Gal-CPCs at DP=250 show an approximate 8-fold enhancement over Co-CPC-20%. This is counter to what has been observed in other glycomacromolecule systems, as well as the general understanding that the dense orientation of glycan ligands limits their accessibility by lectins (Naka et al., 2023, Polymer Journal, 55, 1379). As such, the solution behavior of these materials was investigated using computational models.

### Coarse-Grain Modeling Analyses.

To investigate CPC conformations in solution, coarse-grained (CG) molecular dynamics (MD) simulations were employed, which enabled studying large molecular systems like bottlebrush polymers (BBPs) for several microseconds (Joshi, 2021, Molecular Simulation, 57, 786; Mohammadi et al., 2021, Comp. Mater. Sci., 199, 110720). To probe the effect of grafting density on CPC conformation, CG MD simulations were performed by simulating three types of CPCs with similar grafting densities to those used in present experiments. Here, transferable CG models of peptides (Conway et al., 2020, Mol. Syst. Des. Eng., 5, 675; Mohammadi et al., 2023, Biomacromolecules, 2, 24, 4078), hydrocarbons (An et al., 2018, J. Phys. Chem. B, 122, 7143; An et al., J. Phys. Chem. B., 123, 909), and carbohydrates were utilized to model and simulate CPCs in explicit water. CG MD simulations were carried out for three initial configurations for each system, on the Nanoscale Molecular Dynamics (NAMD) simulation package using the NPT (T=298 K) for 500 ns (Phillips et al., 2005, J. Comp. Chem., 26, 1781). Additional details regarding the mapping schemes, CG models, and simulation details are found in Table 6, Table 7, and Table 8.

TABLE 6
Comparison of bulk properties obtained for β-galactose
and β-glucose using AA and CG simulations.

| | β-galactose | | β-glucose | |
|---|---|---|---|---|
| Property | AA | CG | AA | CG |
| Density (g/cm³) | 1.44 ± 0.00 | 1.49 ± 0.00 | 1.44 ± 0.00 | 1.48 ± 0.00 |
| Heat of vaporization | 29.65 ± 0.19 | 30.49 ± 0.06 | 30.34 ± 0.08 | 30.33 ± 0.03 |
| (kcal/mol) | | | | |
| Surface Tension | 114.05 ± 35.20 | 135.35 ± 9.69 | 124.11 ± 24.92 | 127.75 ± 41.34 |
| (mN/m) | | | | |
| Gibb's solvation-free | 16.78 ± 3.16 | 14.57 ± 0.24 | 16.78 ± 2.65 | 14.65 ± 0.35 |
| energy (kcal/mol) | | | | |

For obtaining bulk properties, systems containing 800 monosaccharide molecules in a cubic box of length 54 Å were made using Packmol (Martinez et al., 2009, J. Comp. Chem., 30, 2157) and simulated under periodic boundary conditions (Allen, Computer Simulation of Liquids, 2017) at 300 K and 1 atm in an NPT ensemble. Single monosaccharides in vacuum cubic boxes were generated and simulated under the same conditions, using an NVT ensemble. For both NPT and NVT ensembles, MD simulations were performed for 20 ns for AA and 50 ns for CG systems. All the AA and CG MD simulations for carbohydrates were performed by using the NAMD 2.14 program (Phillips et al., 2005, J. Comp. Chem., 27, 1781). The last 10 ns (for AA) and 20 ns (for CG) were utilized to calculate the density and heat of vaporization. Density was obtained by dividing the mass of the system by the final equilibrated volume. The heat of vaporization was calculated using the following equation:

$$Δ {H}_{v} ( T ) = \frac{1}{{N}_{m o l}} {E}_{g a s} ( T ) - \frac{1}{{N}_{m o l}} {E}_{l i q} ( T ) + R T$$

Where E_{gas} and E_{liq} are the potential energies of the vacuum and bulk phase, respectively. R is the gas phase constant, T is temperature, and Nₘₒₗ is the number of molecules. To calculate the surface tension, 40 ns (for AA) and 80 ns (for CG) MD simulations were performed, in the NVT ensemble, of monosaccharide slabs by expanding the Z-axis of the cubic cell of equilibrated NPT bulk simulations of 800 molecules to 160 Å (~3 times greater than the X and Y axis). Surface tension was calculated using the following equation:

$$γ = \frac{{L}_{z}}{4} 〈 2 {P}_{zz} - {P}_{x x} - {P}_{y y} 〉$$

where L_{Z} is the box length along the Z-axis, P indicates the diagonal elements of the pressure tensor, and angular brackets denote an ensemble average.

For obtaining Gibbs' solvation-free energies of the monosaccharides, adaptive biasing force (ABF) MD simulations (Mohammadi et al., 2023, Biomacromolecules, 24, 4078; An et al., 2019, J. Phys. Chem. B, 123, 909) were utilized. These simulations were also conducted using the NAMD 2.14 program and the Colvars package. The ABF simulations were performed in the NVT ensemble, where temperature control was achieved in the same manner as for bulk MD simulations. The initial configurations of single monosaccharide solvated in water (TIP3P for CHARMM and GLYCAM, SPCE for OPLS-AA, and CG water beads for CG monosaccharides) were generated using Packmol (Fiorin et al., 2013, Mol. Phys., 111, 3345).

The initial configuration was generated by solvating the monosaccharide in a cubic box of length 40 Å, keeping the monosaccharide in the center of the box. The solvated box contained 2000 AA water molecules and 1000 CG water beads. The air/vacuum-water interface was created by extending the z-axis to 80 Å. The monosaccharide molecule was transported from the center of the solvated box to vacuum by moving it along the z-axis at a 35 Å distance. This 35 Å is referred to as the reaction coordinate, which is a measure of the distance between the center of mass (COM) of the solvated box and that of the molecule in the z-direction. To enhance the efficiency of the ABF simulations for AA systems, the entire reaction coordinate was equally divided into seven consecutive windows, and 20 ns MD simulation was performed at each window. For CG systems, one 100 ns ABF MD simulation was performed. The Gibbs solvation-free energy profile was obtained along the z-axis from the COM of the box to 35 Å in the vacuum.

Force-field files used to perform CG MD simulations along with the NAMD input files are provided as supplementary files and the corresponding force-field (FF) parameters are shown in Table 7.

TABLE 7
List of all FF parameters used for this study.
Bonded interaction parameters

| Bond | K_{b} | b₀ |
|---|---|---|
| TOL4 TOL4 | 36.609 | 2.52 |
| TOL4 C2M | 50.014 | 2.42 |
| C2M COO2 | 40.016 | 2.66 |
| CON2 CCOH | 38.000 | 3.00 |
| C2M CON2 | 37.695 | 3.561 |
| C2M DBC | 37.695 | 3.561 |
| DRO DGC | 160.000 | 1.86 |
| DRO FSB | 160.000 | 1.86 |
| DBC FSB | 90.000 | 2.2250 |
| DGC FSB | 90.000 | 2.2250 |
| DGC DBC | 100.000 | 2.2506 |
| DBC DBC | 100.000 | 2.2506 |
| DBC DBB | 50.000 | 2.8645 |
| DRO DBB | 50.000 | 2.110 |
| DBC FSB | 100.000 | 2.00 |

| Angle | K_{Θ} | Θ₀ |
|---|---|---|
| TOL4 TOL4 TOL4 | 15.000 | 144.226 |
| TOL4 TOL4 C2M | 15.000 | 100.000 |
| TOL4 C2M COO2 | 15.000 | 120.000 |
| C2M COO2 C2M | 15.000 | 162.000 |
| CON2 C2M DBC | 15.000 | 162.000 |
| CON2 C2M COO2 | 15.000 | 162.000 |
| C2M CON2 C2M | 15.000 | 162.000 |
| C2M DGC DRO | 16.555 | 124.000 |
| DGC DGC DRO | 100.000 | 110.000 |
| DBC DBC DGC | 100.000 | 110.000 |
| DBC DBC DBB | 90.000 | 110.000 |
| DBC DBB DRO | 100.000 | 110.000 |
| DGC DRO DBB | 80.000 | 110.000 |
| FSB DRO DBB | 80.000 | 110.000 |
| DBC DGC FSB | 100.000 | 110.000 |
| DGC FSB DRO | 100.000 | 110.000 |
| DBB DBC FSB | 100.000 | 123.000 |
| FSB DBC DBC | 100.000 | 114.000 |
| DRO FSB DBC | 100.000 | 115.000 |
| DGC FSB DBC | 100.000 | 130.000 |

| Dihedral | K_{φ} | n | φ₀ |
|---|---|---|---|
| TOL4 TOL4 TOL4 TOL4 | 1.0 | 2.0 | 0.0 |
| TOL4 TOL4 TOL4 C2M | 1.0 | 2.0 | 60 |
| TOL4 TOL4 C2M COO2 | 1.0 | 2.0 | 0.0 |
| C2M TOL4 TOL4 C2M | 1.0 | 2.0 | 0.0 |
| DRO DBB FSB DRO | 0.5 | 3.0 | 0.0 |
| DGC DBB FSB DRO | 0.5 | 2.0 | −120.0 |
| DBC DGC DBC DBC | 1.0 | 1.0 | 53.5 |
| DBC DBC DBC DGC | 1.0 | 1.0 | 53.5 |
| DBC DBC DGC DBB | 1.0 | 1.0 | −53.5 |
| DGC DBB DRO FSB | 1.0 | 1.0 | −60.0 |
| DBB DRO FSB DGC | 1.0 | 1.0 | 60.0 |
| DGC DBC DGC DSB | 1.0 | 1.0 | 53.5 |
| DRO DGC DBC DBC | 1.0 | 1.0 | 53.5 |

Nonbonded interaction parameters

| Self-interactions | Epsilon | Rₘᵢₙ/2 |
|---|---|---|
| DGC | −0.6200 | 1.9700 |
| DBC | −0.6200 | 1.9700 |
| DBB | −1.1600 | 2.2740 |
| DMB | −0.3710 | 2.4354 |
| DSB | −0.2700 | 1.9955 |
| FSB | −0.2700 | 1.9955 |
| DRO | −0.3650 | 1.9955 |
| CON2 | −1.1332 | 2.4343 |
| COO2 | −0.8714 | 2.3950 |
| TOL4 | −0.5839 | 2.6920 |
| CCOH | −0.7999 | 2.3741 |
| C2M | −0.3420 | 2.4343 |
| W | −1.1425 | 2.1170 |
| Cross interactions | Epsilon | Rₘᵢₙ |
| DBB - W | −0.780000 | 4.250000 |
| DMB - W | −0.716448 | 4.109073 |
| DRO - W | −0.620000 | 4.523235 |
| DGC - W | −0.700000 | 4.250000 |
| DBC - W | −0.670000 | 4.350000 |
| DSB - W | −0.630000 | 4.050000 |
| C2M - W | −0.440000 | 4.235900 |
| CON2 - W | −1.095258 | 4.5513 |
| CCOH - W | −0.798158 | 4.4911 |
| COO2 - W | −1.056721 | 4.512 |

MD simulations were carried out using the NAMVD simulation package for 500 ns with periodic boundary conditions. The NPT ensemble with T=298 K, and P=1 bar was used for the production run. A timestep of 10 fs was used with a cut-off of 12 Å. Visual Molecular Dynamics (VIVD) software was used for visualization and analysis of trajectories (Humphrey et al., 1997, J. Molec. Graphics, 14, 33). Furthermore, to obtain more statistically significant results, each system was simulated thrice using different initial configurations (Table 8). Entire trajectories from all simulations were used to calculate the evolution of R_{g} and solvent accessible surface areas (SASA). However, aspect ratios, distributions and radial distribution functions (RDFs) were analysed for the last quarter of the trajectories (150 ns), when all systems were considered to have been equilibrated (Joshi et al., 2022, Comp. Mater., 8, 45; Joshi et al., 2021, Carbon, 180, 244). Analyses were carried out using the CPPTRAJ package and in-house TCL and Python scripts (Roe, 2013, J. Chem. Theo. Comp., 9, 3084).

TABLE 8
List of all CG MD simulations performed.

| System bundle | Systems studied, replicas | Total run time |
|---|---|---|
| Co-CPC-50% | 3 (different starting | 1.5 μs |
| | configurations; 500 ns each) | |
| Gal-CPC | 3 (different starting | 1.5 μs |
| | configurations; 500 ns each) | |
| Lac-CPC | 3 (different starting | 1.5 μs |
| | configurations; 500 ns each) | |

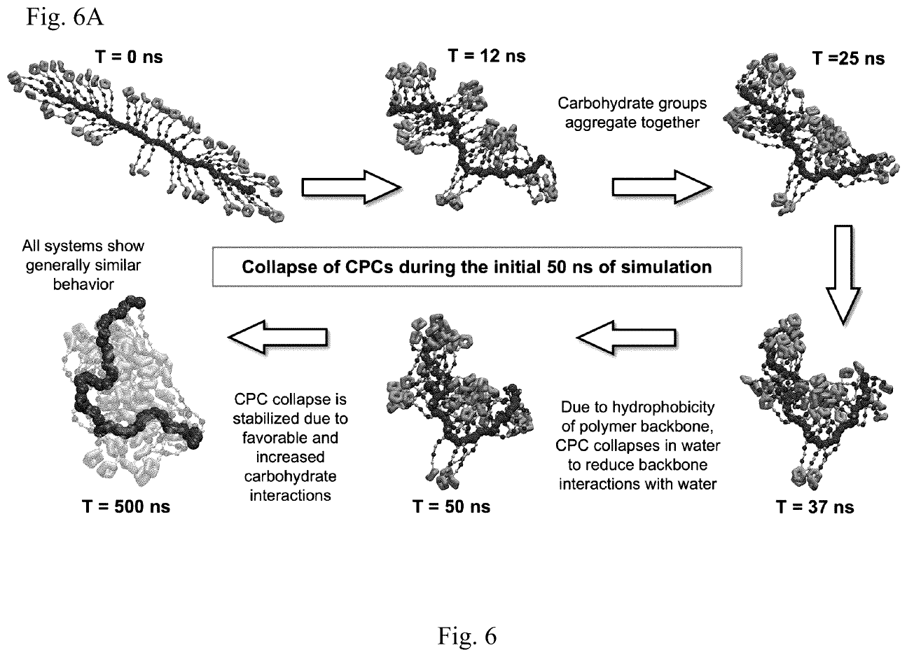

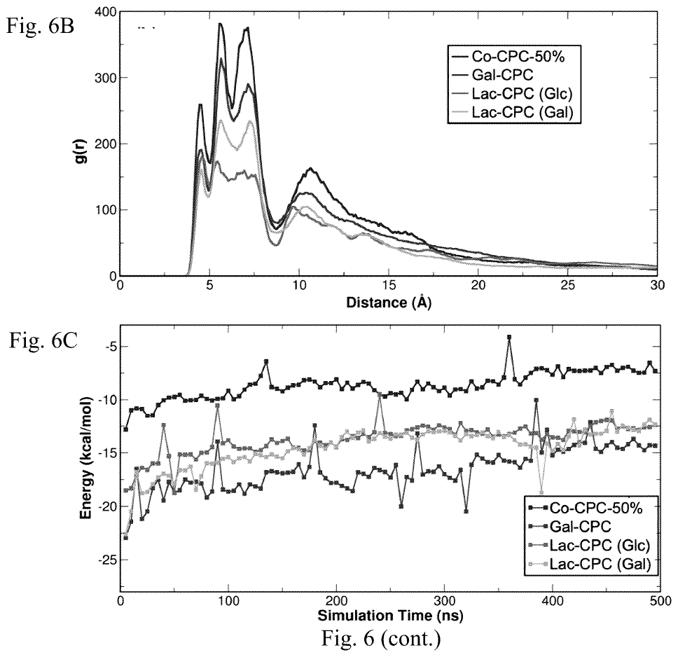

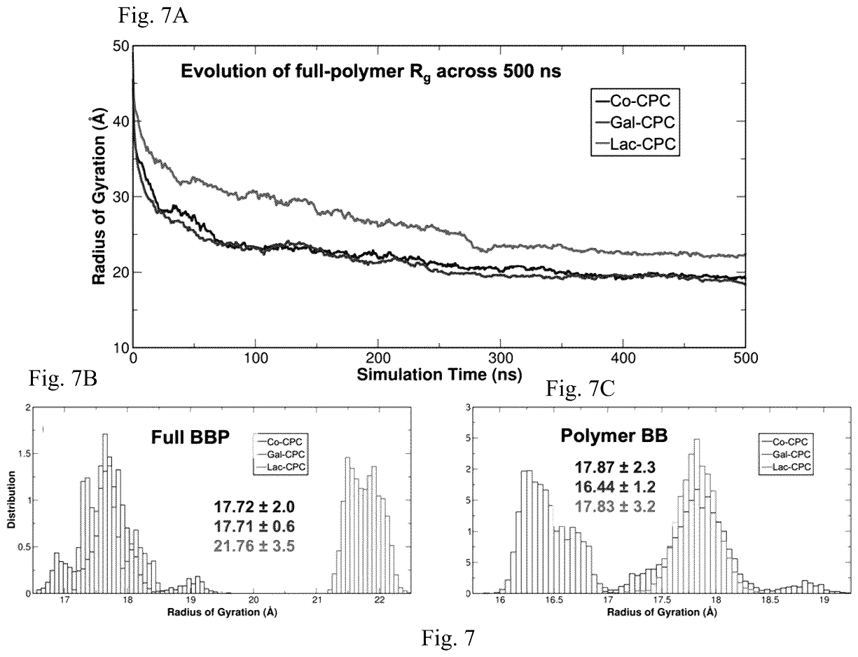

Visual inspection of CG MD simulations trajectories revealed that all CPCs collapsed into globular aggregates within the initial 100 ns (FIG. 6A). It was observed that the monosaccharide functional groups aggregated together initially, followed by the collapse of the hydrophobic CPC backbones to reduce backbone interactions with water. This collapse of the CPC backbone was simultaneously stabilized through the increased carbohydrate aggregation, which resulted in equilibrated globular structures with aspect ratios of 0.68±0.12, 0.71±0.10, and 0.69±0.10 for Lac-CPC, Gal-CPC, and Co-CPC-50% systems, respectively. Additionally, following equilibration, R_{g} values for Lac-CPC systems fluctuated around a mean value of 2.176±0.35 nm compared to 1.771±0.06 nm and 1.772±0.2 nm of Gal-CPC and Co-CPC-50% systems, which were much lower than the R_{g} values of stretched CPCs (FIG. 7). Based on aspect ratios and R_{g} values for these globule-like structures, the relation R_{g}/Rₕ≈0.78 was utilized to estimate the hydrodynamic radii (Rₕ) of the present CPCs (Hsiao, 2006, Macromolecules, 39, 7125; Nygaard et al., 2017, Biophys. J., 113, 550). These values for the Lac-CPC, Gal-CPC, and Co-CPC-50% systems were 2.789 nm, 2.271 nm, and 2.272 nm, respectively. These data were in good agreement with experimental Rₕ values, measured via dynamic light scattering (DLS, Table 9), for Lac-CPCs (Rₕ=3.35 nm), Gal-CPCs (Rₕ=2.55 nm) and the statistical copolymer (Rₕ=2.45 nm).

TABLE 9
Polymer size characterized via dynamic light scattering.

| Polymer | Size (d · nm)* | Standard Deviation (d · nm)* |
|---|---|---|
| Entry 15 | 5.14 | 0.14 |
| Entry 16 | 6.77 | 0.74 |
| Entry 17 | 4.89 | 0.46 |
| Entry 18** | 9.22 | 0.34 |
| Entry 19** | 8.08 | 0.74 |
| Entry 20** | 5.31 | 0.25 |

*Calculated using percent number.
**Measurement was run in 1X PBS

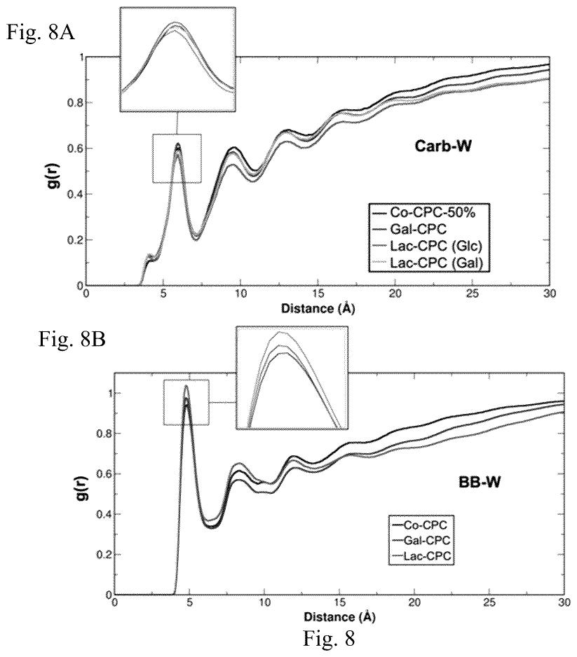

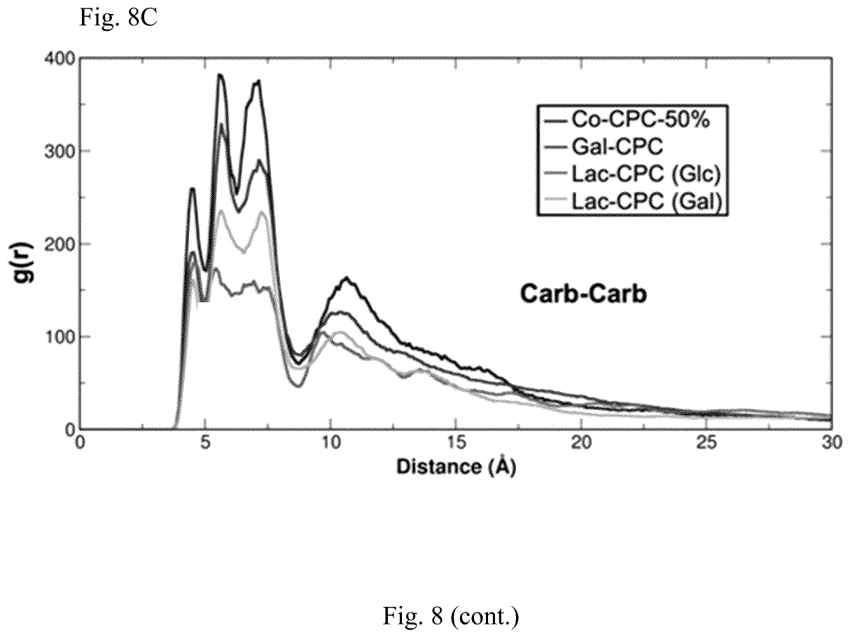

To examine microscopic structural correlations in the system, the radial distribution functions (RDFs) were calculated between different bead types (FIG. 8). RDFs between CPC backbone and water showed greater hydration of the Lac-CPC system, followed by the Gal-CPC and Co-CPC-50% systems. Similarly, RDFs between monosaccharide groups in the CPCs with water showed similar structural correlations with slightly greater hydration for the monosaccharide groups in the Lac-CPC system. RDFs between monosaccharide groups that are known to be important for binding to Gal-3 showed that the structural correlation between monosaccharide groups was highest for Co-CPC-50% systems compared to homopolymers Lac-CPC and Gal-CPC (FIG. 6B). These data indicate that the homopolymer systems show more exposure of monosaccharide groups to the solvent, whereas for the Co-CPC-50% system, monosaccharide groups are more strongly aggregated.

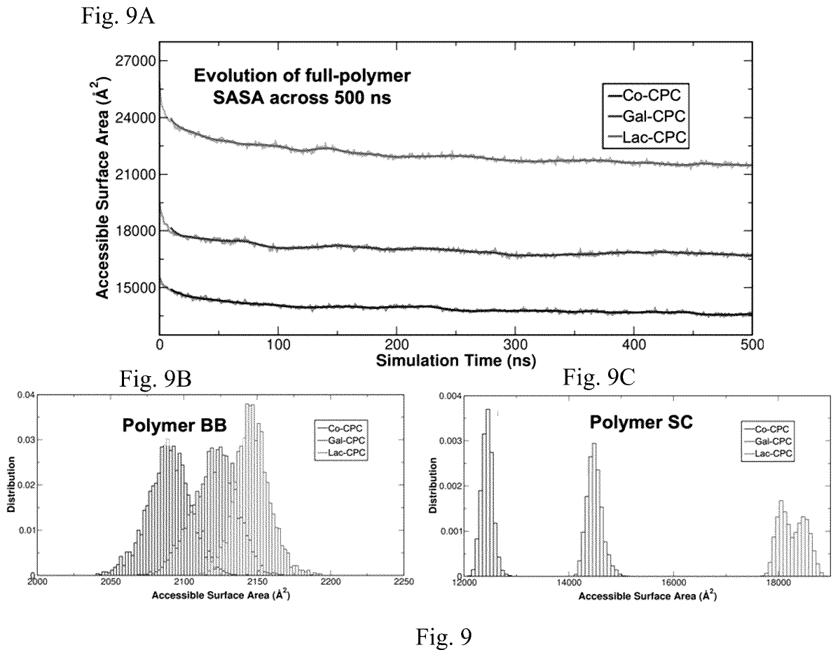

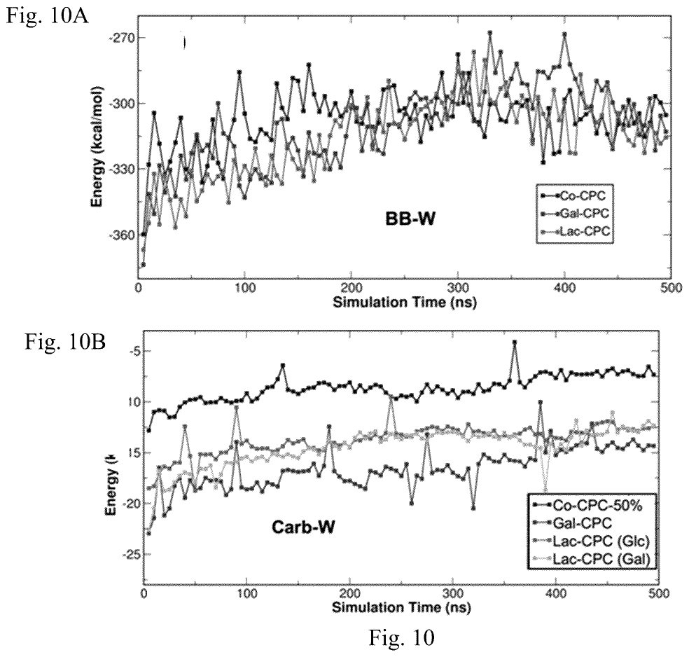

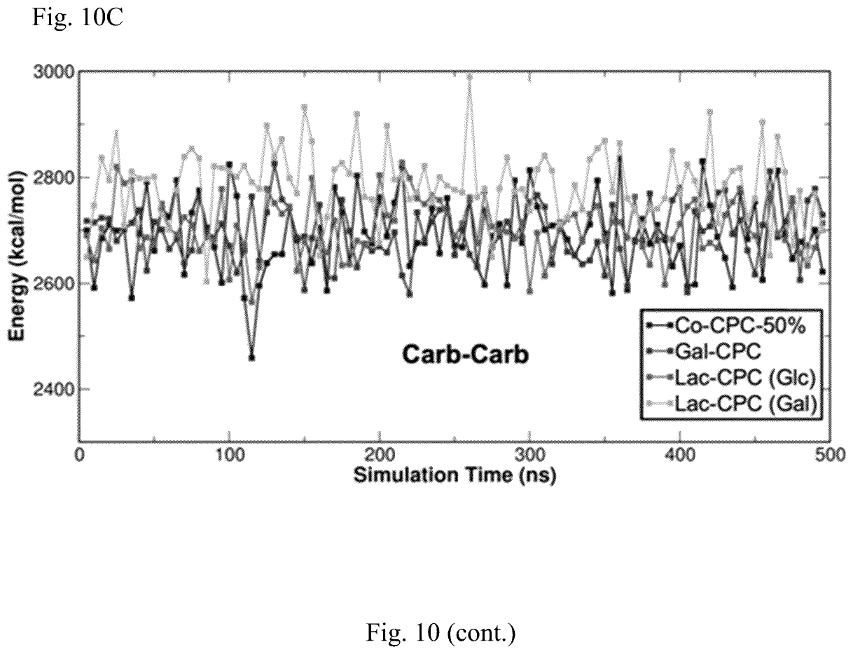

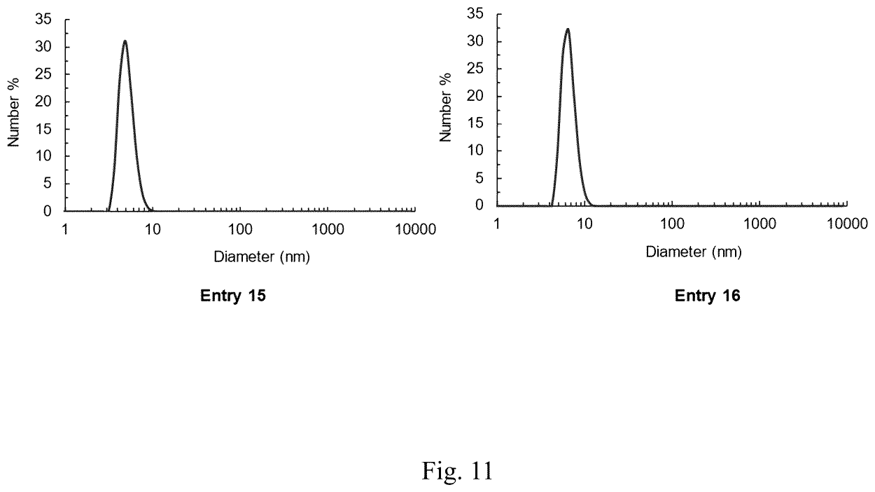

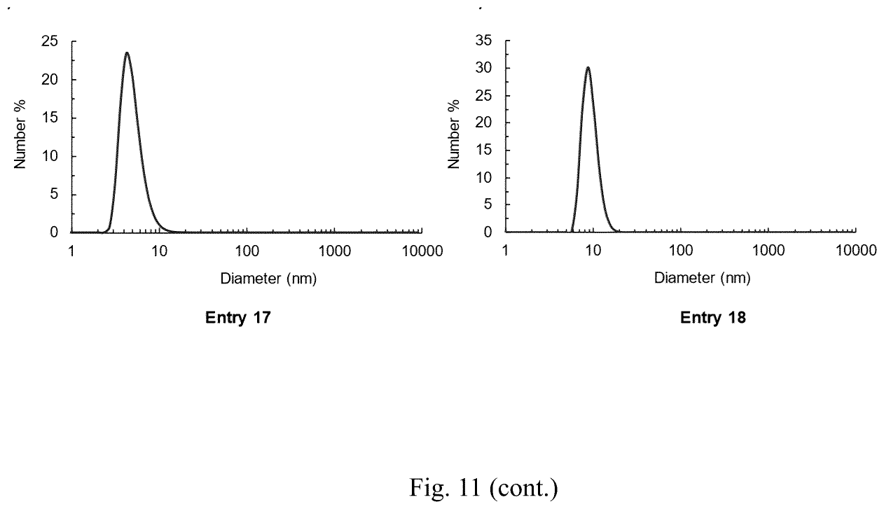

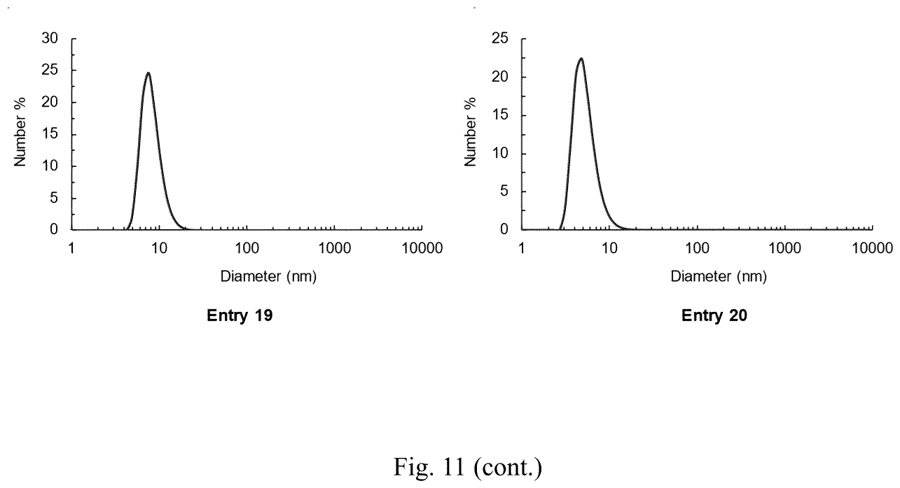

Structural correlations between water and monosaccharide groups follows the order: Lac-CPC>Gal-CPC>Co-CPC-50%. Similarly, the solvent accessible surface areas (SASA) values for all systems were calculated, showing the highest values for the Lac-CPC system followed by Gal-CPC and Co-CPC-50% systems (FIG. 9). This could be the result of stronger aggregation between monosaccharide groups in Co-CPC-50% compared to the rest, thus further validating the RDF data. Additionally, the nonbonded energies between different groups in the systems were isolated and obtained for the entire trajectory (FIG. 10). While the nonbonded energies between backbone-water and between monosaccharide groups showed similar values in all CPC systems, a clear difference was observed for energies between monosaccharide groups and water (FIG. 6C). The interaction energy with water was found to be lowest with monosaccharide groups in the Gal-CPC system, indicating the most favorable interactions. This was followed by the monosaccharide groups in the Lac-CPC system, whereas the Co-CPC-50% system exhibited the most unfavorable interactions with water. These data complement prior observations indicating that monosaccharide groups in Gal-CPC and Lac-CPC systems show more favorable interactions with water, thus making them available for binding to Gal-3. Moreover, these data are supported experimentally via dynamic light scattering (DLS, FIG. 11), with Lac-CPCs having the largest hydrodynamic diameter (6.7 nm), followed by Gal-CPCs (5.1 nm) and finally the statistical copolymer (4.9 nm).

### Cellular Uptake and Cytotoxicity of CPCs.

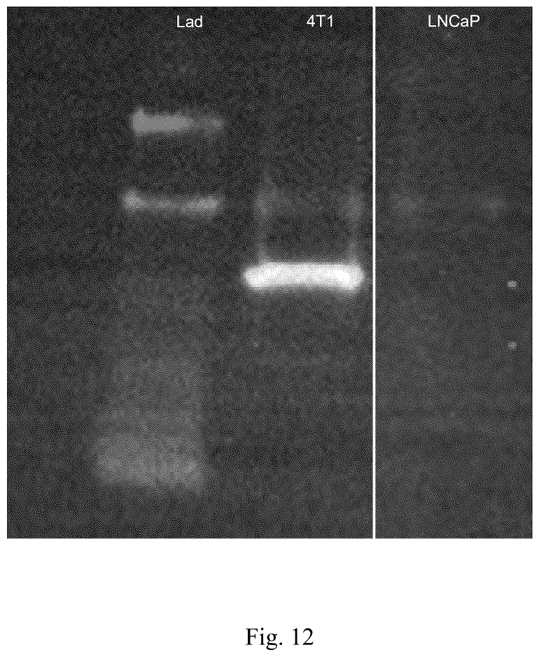

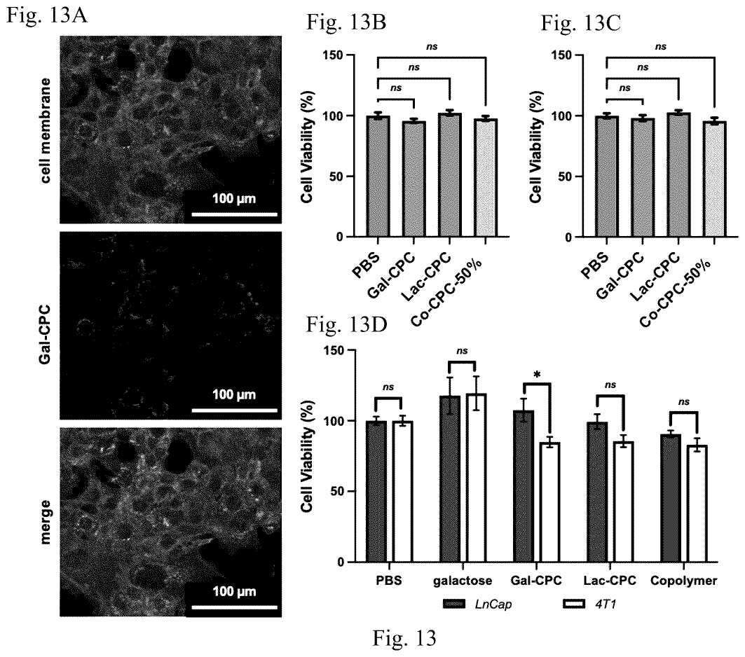

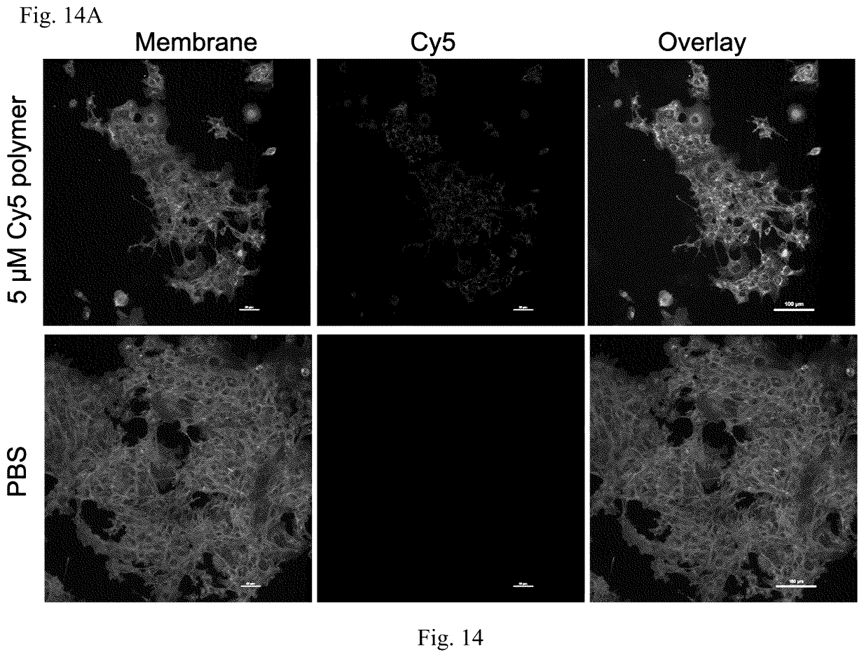

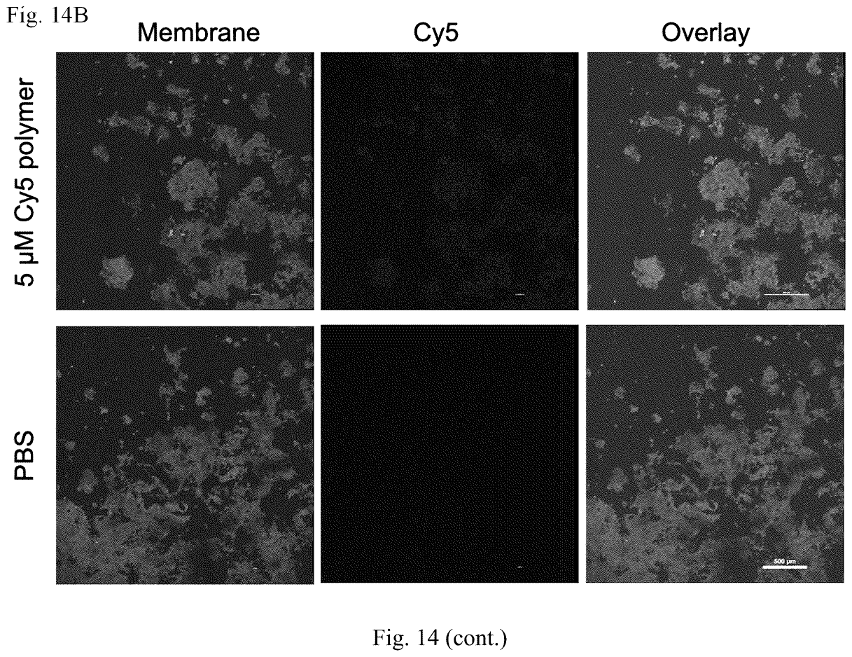

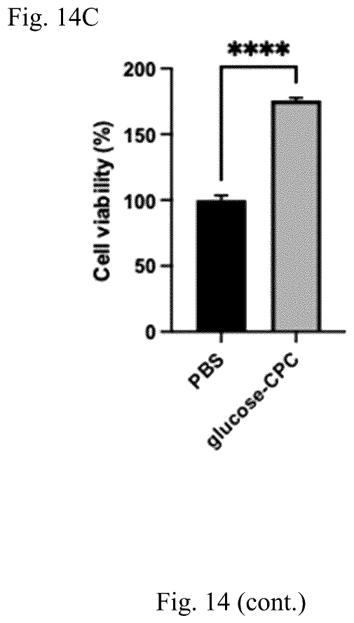

To evaluate whether differences in binding avidity and solution behavior also translates to differences in biological function, the ability of CPCs to be taken up by cells was first evaluated. As a model system, Gal-CPCs containing a near infrared (NIR) fluorophore (Proetto et al., 2018, ACS Cent. Sci., 4, 1477) were synthesized (FIG. 12) and their uptake by 4T1 triple negative breast cancer cells was evaluated. Gal-CPCs were efficiently taken up by cells (FIG. 13A) and appear to localize around the exterior of the nucleus. It was then confirmed that all CPCs were not acutely cytotoxic in both a Gal-3-negative cell line (LNCaP) and a Gal-3-positive cell line (4T1), as confirmed by Western Blot (FIG. 14). No system showed evidence of cytotoxicity after 4 h incubation at polymer concentrations of 10 μM (FIG. 13B and FIG. 13C).

### In Vitro Anticancer Activity as a Function of Glycan Display.

Among its multiple tumorigenic activities, intracellular Gal-3 is known to promote both cancer cell proliferation and migration. As such, it was important to understand whether CPCs could inhibit cell proliferation and migration, and whether the differences in in vitro binding avidity translates to appreciable differences in biological activity.

To assess the effect of structure on Gal-3-promoted cell proliferation, both 4T1 cells and LNCaP cells were incubated with Gal-CPCs (DP=100), Lac-CPCs (DP=100), or Co-CPC-50% at 10 μM with respect to polymer. As a control, cells were incubated with galactose at 100 μM. The effect of treatment on cell proliferation was evaluated by comparing the cell viability of 4T1 cells to those of LNCaP cells after 24 h incubation. As expected, no effect on LNCaP cell proliferation was observed in any system. However, Gal-CPCs significantly inhibited proliferation of 4T1 cells compared to LNCaP cells (FIG. 13D) by approximately 20%, suppressing the growth of cancer cells in a galectin-3-dependent manner. Conversely, Co-CPC-50% was unable to significantly suppress proliferation, indicating that full grafting density is necessary for optimal biological function.

It was noted that Lac-CPCs trend towards being antiproliferative, but Gal-CPCs outperformed these materials. This discrepancy was attributed to the fact that lactose is comprised of galactose bound to glucose by a β-1,4-glycosidic linkage. As such, incubation of Lac-CPCs with cancer cells may inadvertently liberate glucose when in the biological milieu, which has been seen to promote cell growth in glucose-based CPCs (FIG. 14). This underscores the importance of considering both glycan composition, as well as presentation, when designing carbohydrate-based therapeutics.

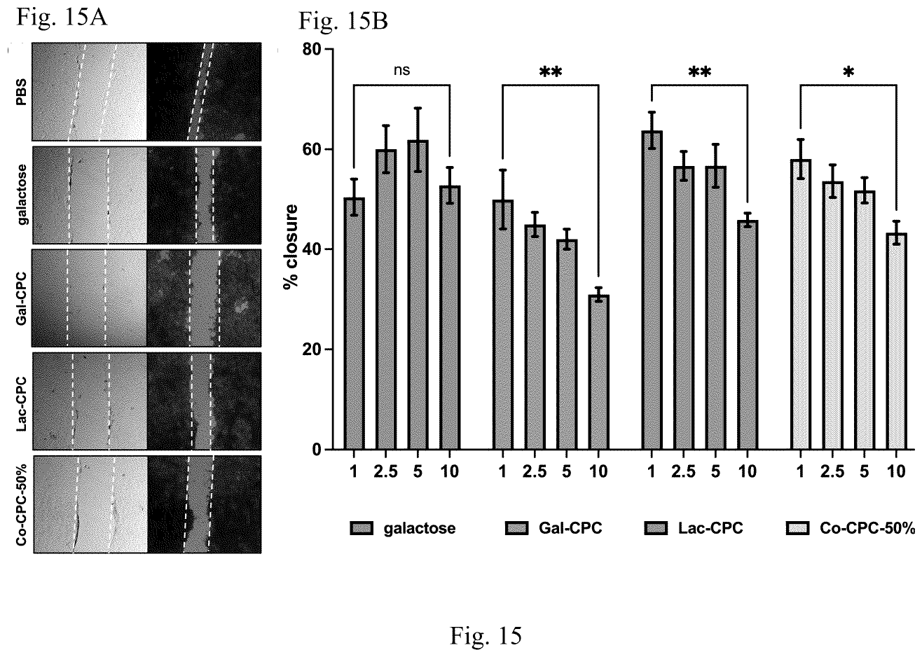

As a final analysis of the effect of glycan macromolecular structure on biological function, the ability of the materials to inhibit cancer cell migration was evaluated. The MAPK/ERK signaling pathway plays a crucial role in cancer cell motility, contributing to the invasive metastatic phenotype of cancer cells. Given the involvement of Gal-3 in the MAPK/ERK signaling pathway and influence on cancer cell motility (Vrbata et al., 2022, J. Med. Chem., 65, 3866), the ability of these materials to inhibit the migration of 4T1 cells was evaluated. A wound-healing (scratch) assay (Liang et al., 2007, Nat. Protocols, 2, 329; Martinotti, 2020, Epidermal Cells; Methods and Protocols, pp 225-229) was conducted on confluent 4T1 cells incubated with Gal-CPCs (DP=100), Lac-CPCs (DP=100), or Co-CPC-50% at 10, 5, 2.5, or 1 μM with respect to polymer. As controls, an additional set of cells was incubated with either PBS or galactose at 100 μM. After 24 h incubation, cell migration was assessed by measuring the extent to which the scratch closed (FIG. 15A). All polymer systems showed a concentration-dependent ability to inhibit 4T1 cell migration (FIG. 15B). However, as was observed in the antiproliferation experiments, Gal-CPCs significantly outperformed all other treatment groups and was able to reduce cancer cell migration by 60%, relative to PBS controls (FIG. 15C) at the highest concentration tested. In addition, Gal-CPCs were the only system able to significantly suppress cancer cell migration at 5 μM (FIG. 15D) and 2.5 μM (FIG. 15E).

### Example 2: Effect of Polymer Backbone Stereochemistry on CPC Hierarchical Assembly and Biological Function

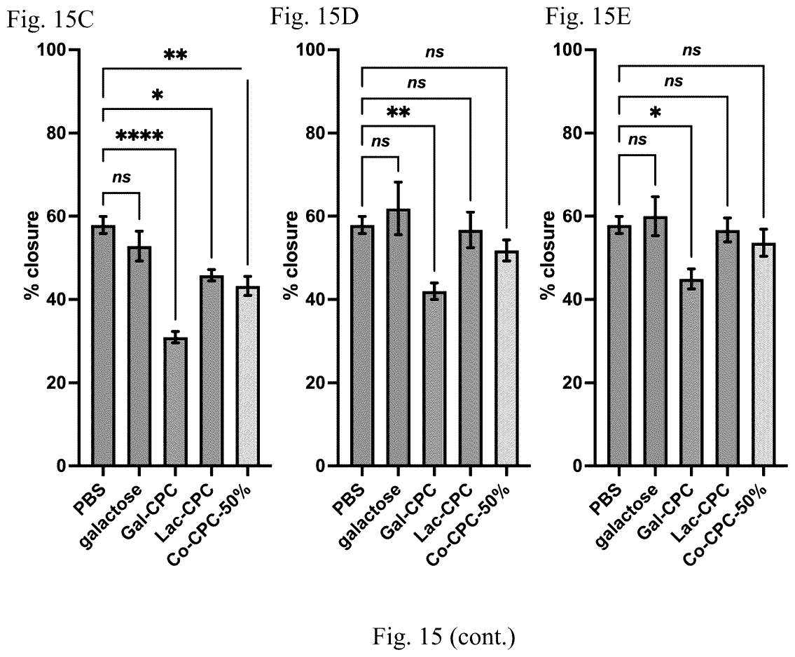

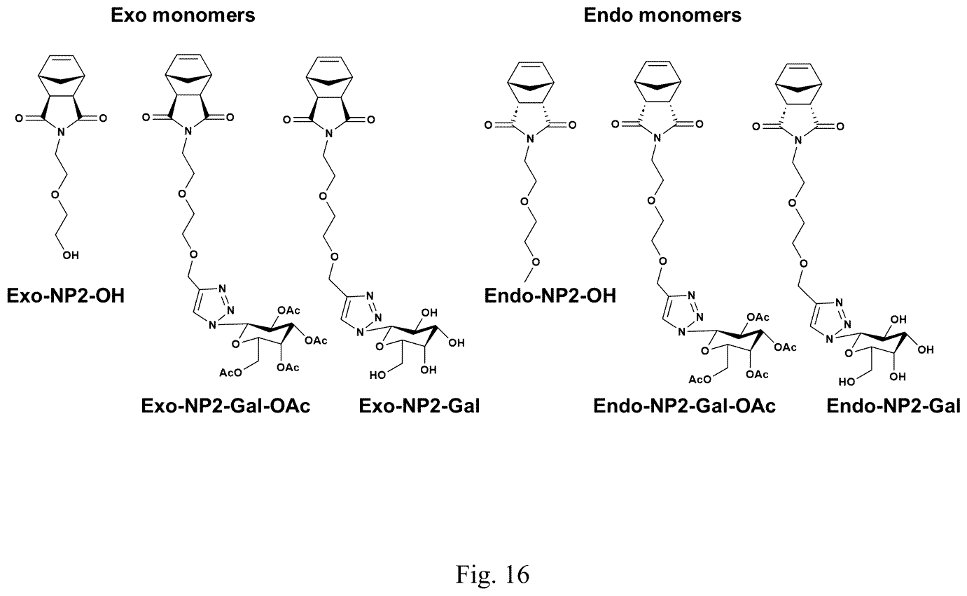

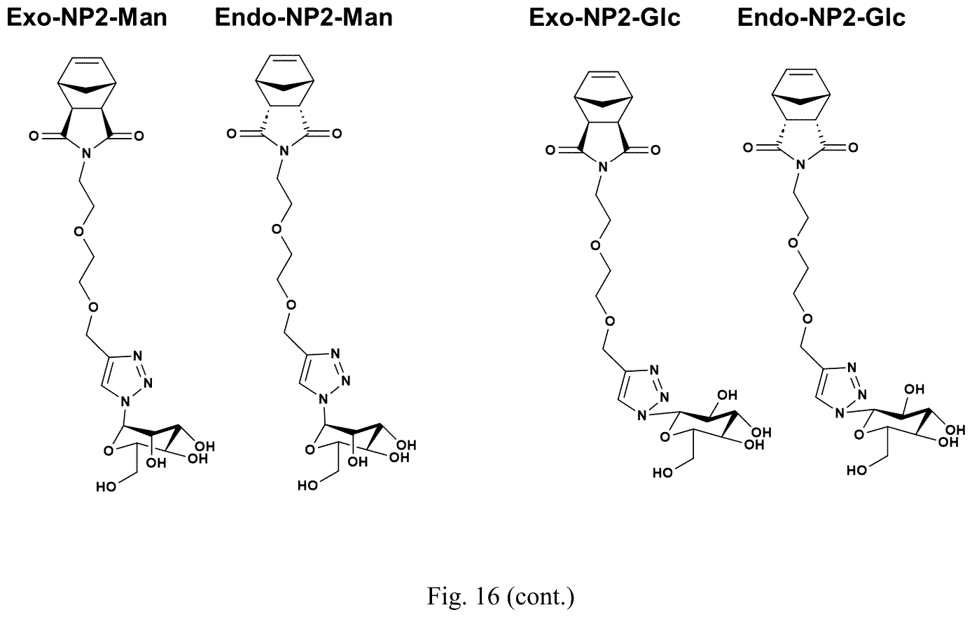

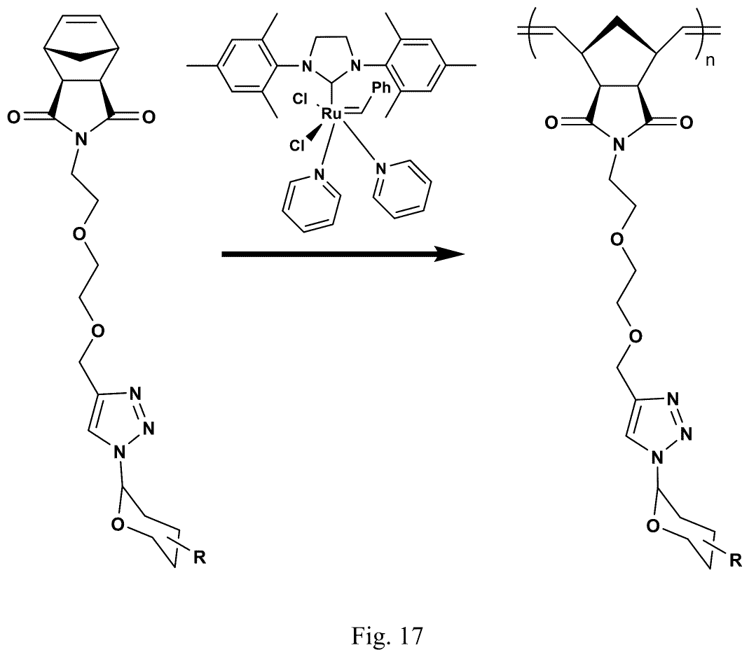

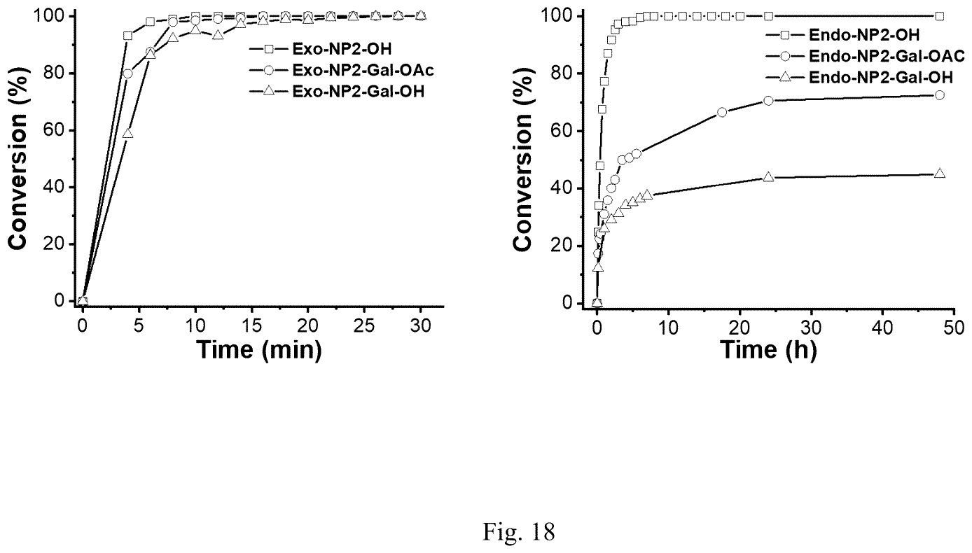

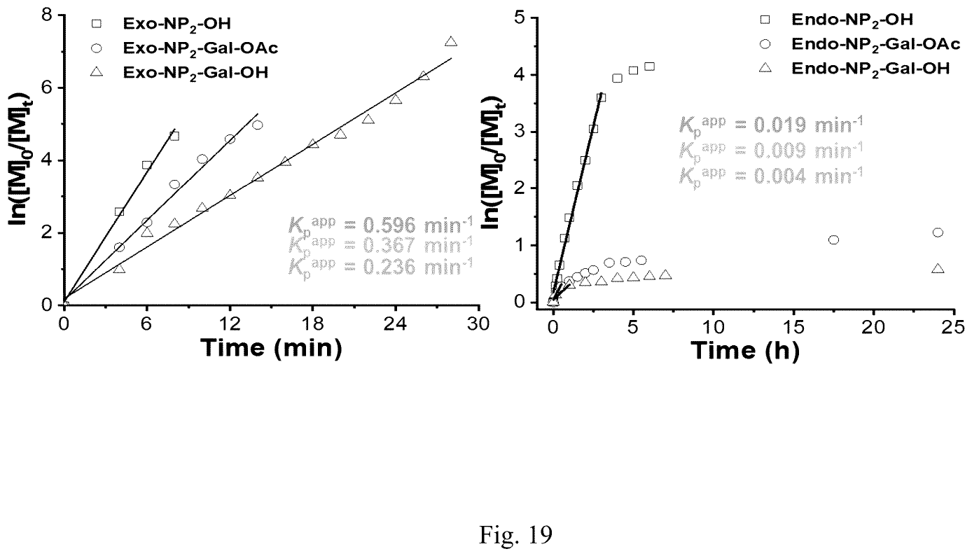

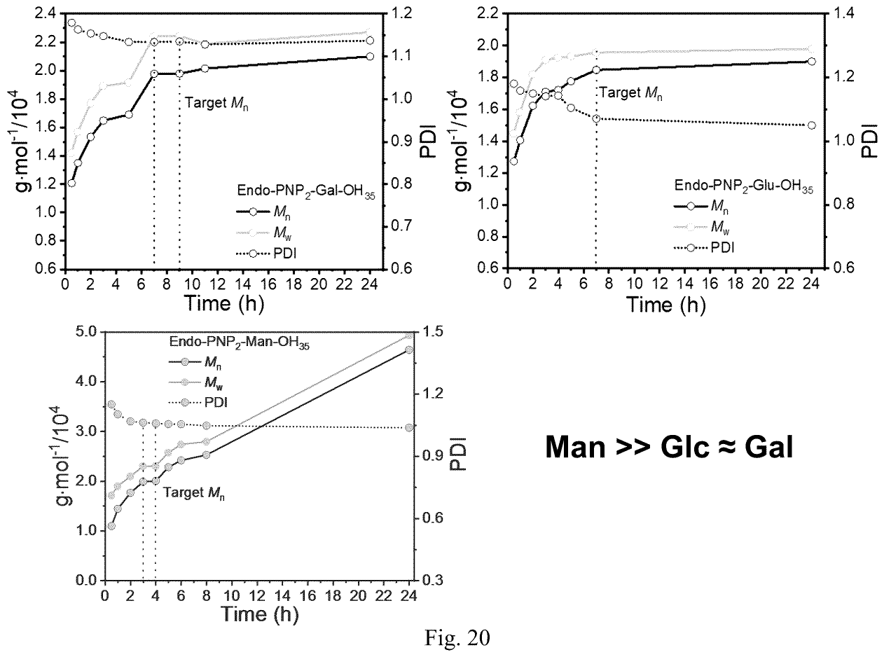

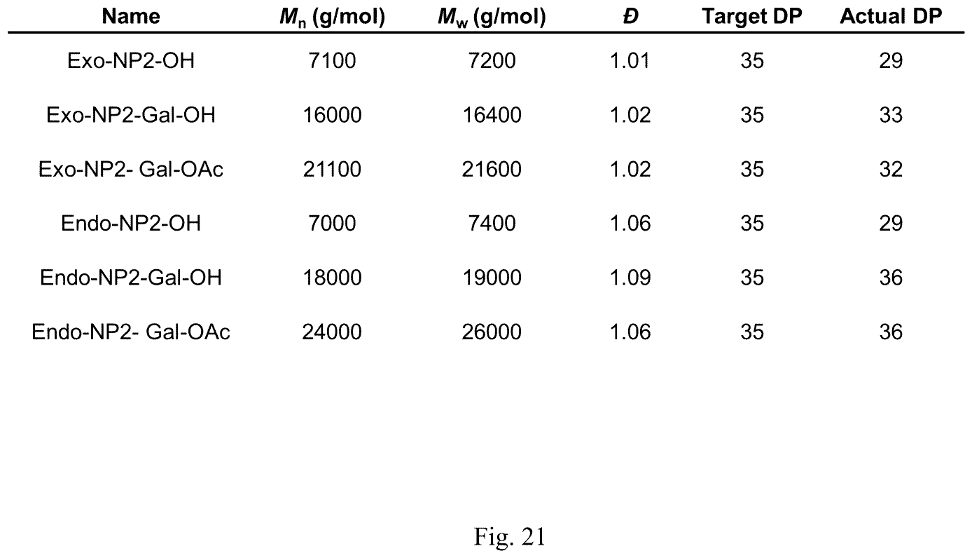

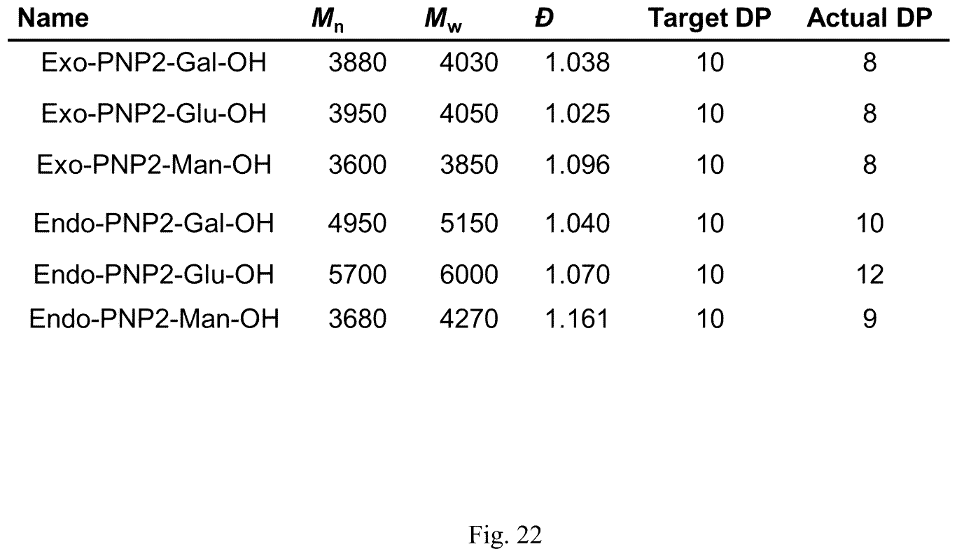

It was found at robust, low dispersity ROMP polymers can be made from all three sugars (mannose, galactose, and glucose) using both exo- and endo-norbornene (FIG. 16, FIG. 17). Polymerization kinetic studies showed exo monomers rapidly and completely polymerize, while endo monomers convert slowly and only the —OH derivative reaches full conversion (FIG. 18, FIG. 19). Glucose (Glc) and galactose (Gal) polymerize at approximately the same rate, and mannose polymerizes significantly faster than Glc and Gal (FIG. 20, FIG. 21, FIG. 22).

Endo-norbornene polymers behave vastly differently from exo-norbornene polymers which is unintuitive. Exo polymers bind proteins more tightly and more efficiently than endo polymers, whereas endo polymers enter cells much more efficiently than exo polymers, and exo polymers are able to capture Nile Red more efficiently, indicating they have more exposed hydrophobic pockets.

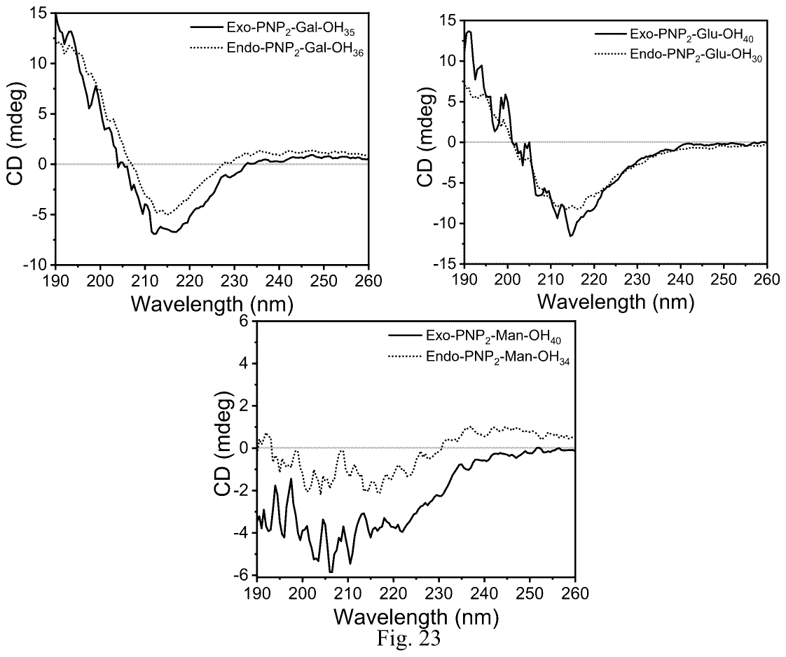

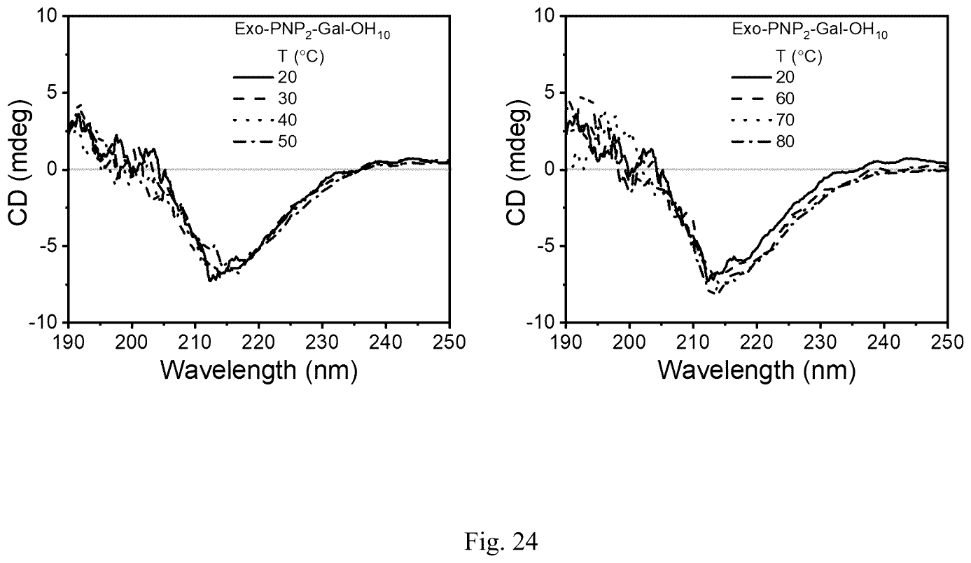

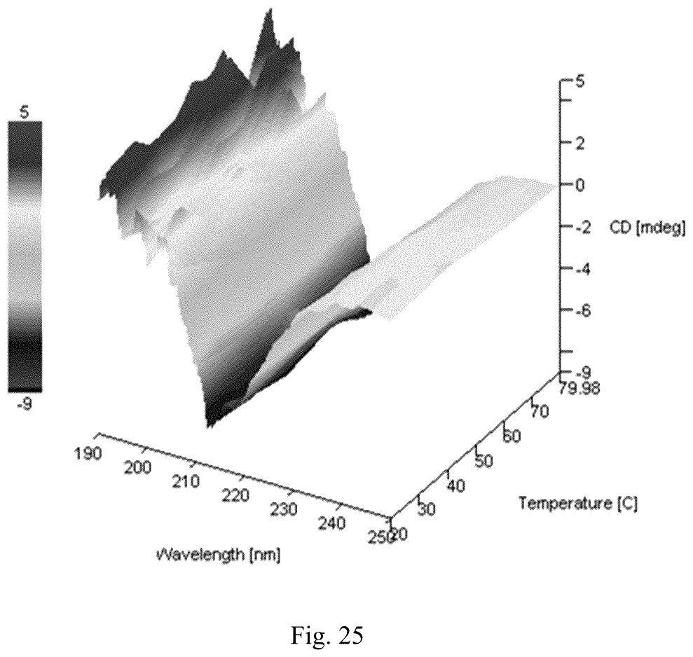

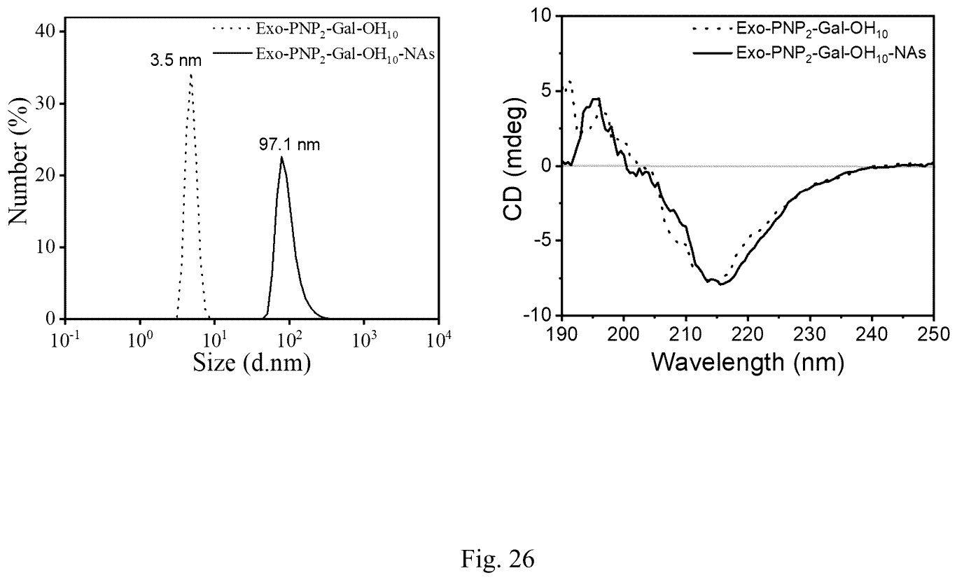

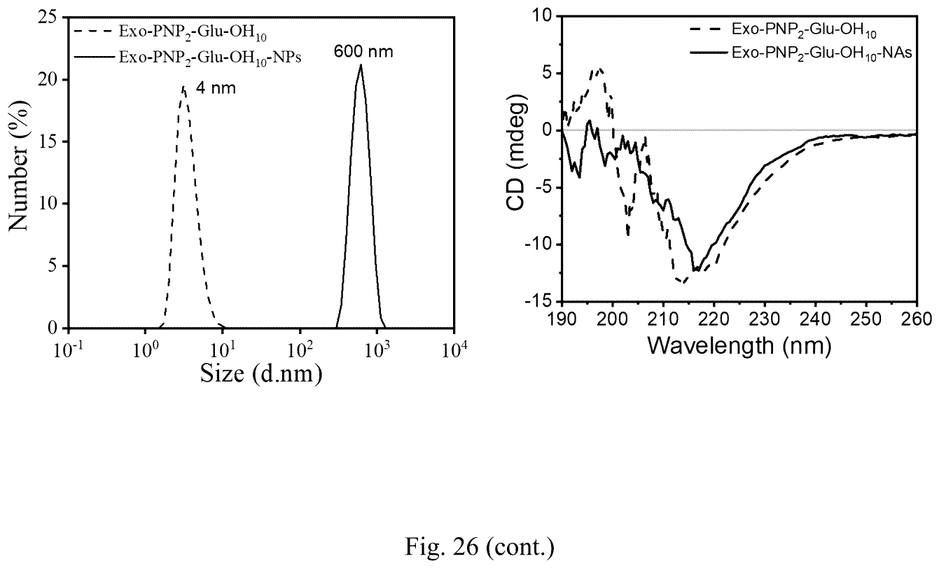

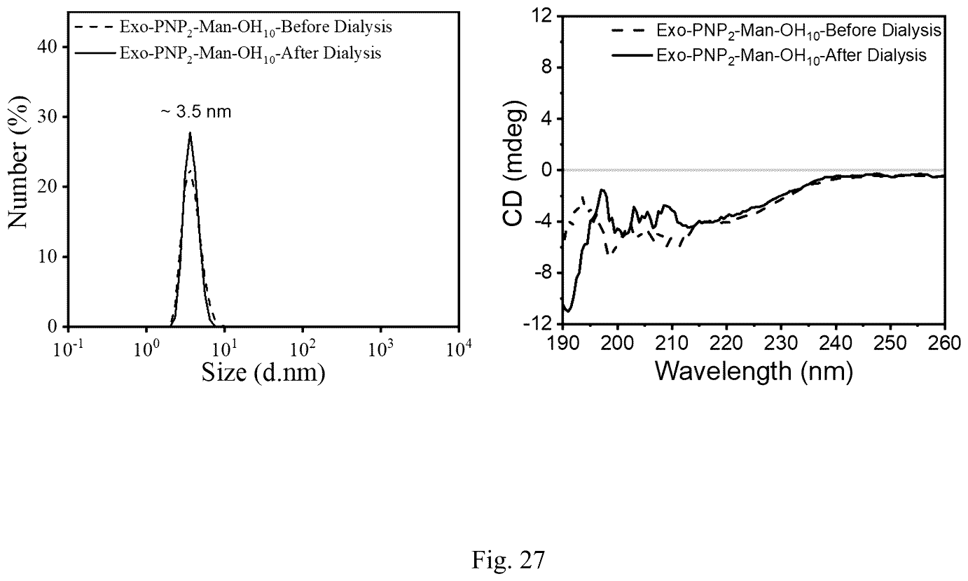

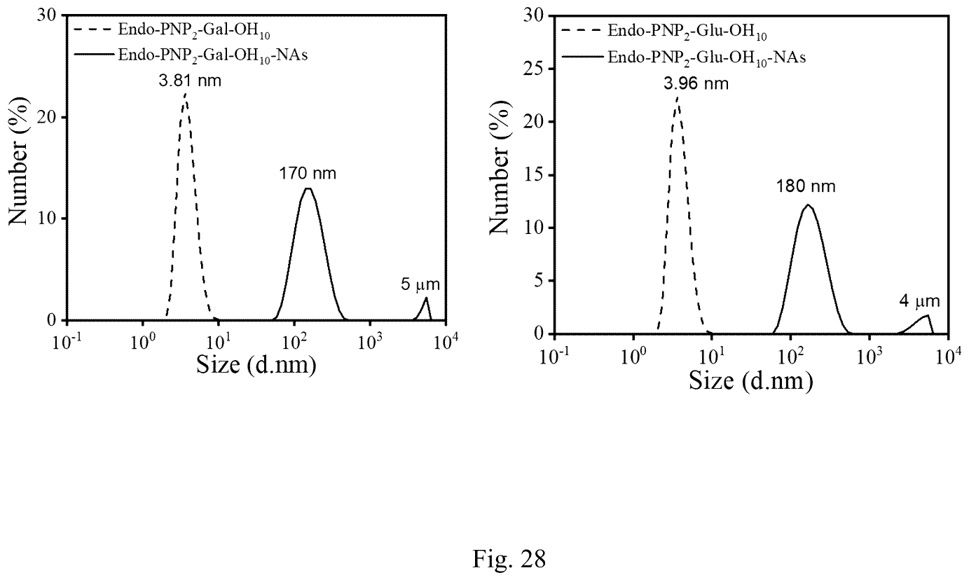

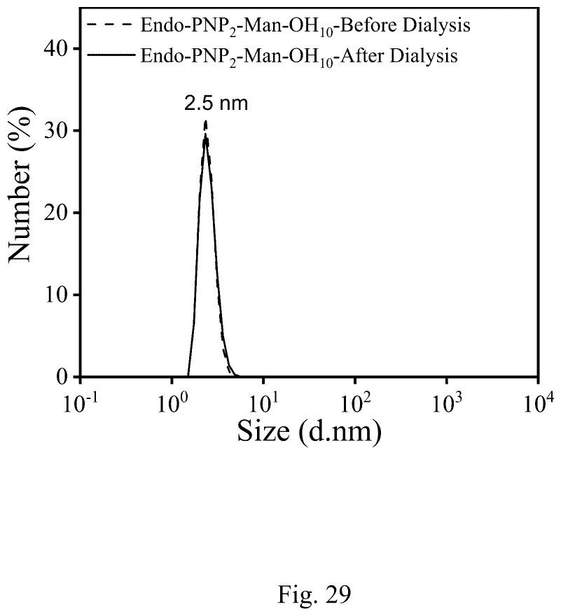

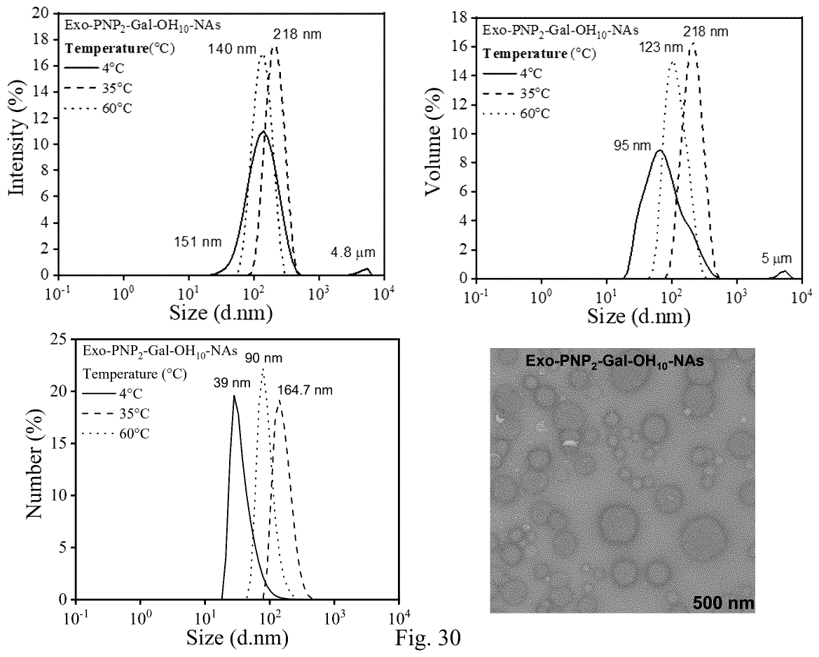

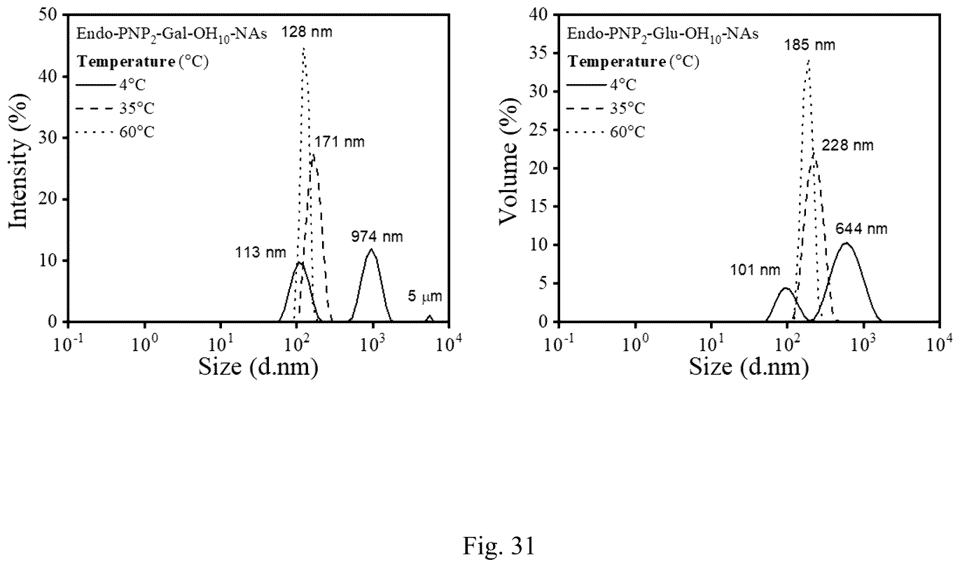

It was found that there was also a dependence on sugar, which is unintuitive, wherein galactose and glucose-based polymers showed secondary structures by circular dichroism (CD), but mannose-based polymers do not (FIG. 23). CD spectra also showed evidence of secondary structures that were temperature dependent (FIG. 24, FIG. 25). Galactose and glucose-based polymers form higher order assemblies following dialysis (FIG. 26). When dissolved in water, polymers remain unimeric in solution, but when dialysed from DMSO into water, polymers form higher order assemblies (“nanoassemblies”) with slightly altered CD structures. Mannose polymers do not form higher order assemblies, even with dialysis (FIG. 27). Endo-Gal and Endo-Glc demonstrate similar behavior (FIG. 28), in contrast with Endo-Man (FIG. 29). Exo-PNP-Gal nanoassemblies (NAs) also showed temperature-responsive behavior. As the temperature increased, the size of the assembly first increases, then decreases (FIG. 30) Exo-PNP-Glc NAs also showed temperature-responsive behavior (FIG. 31).

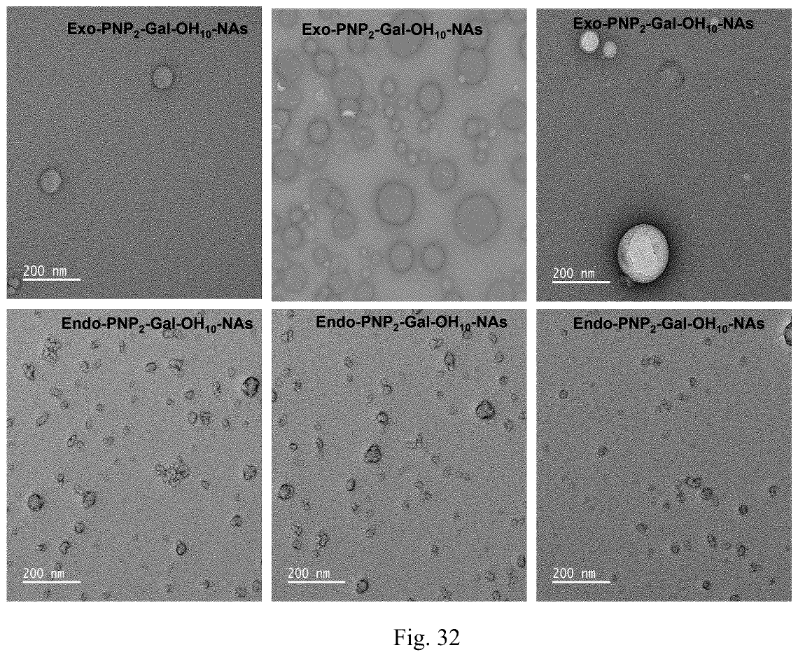

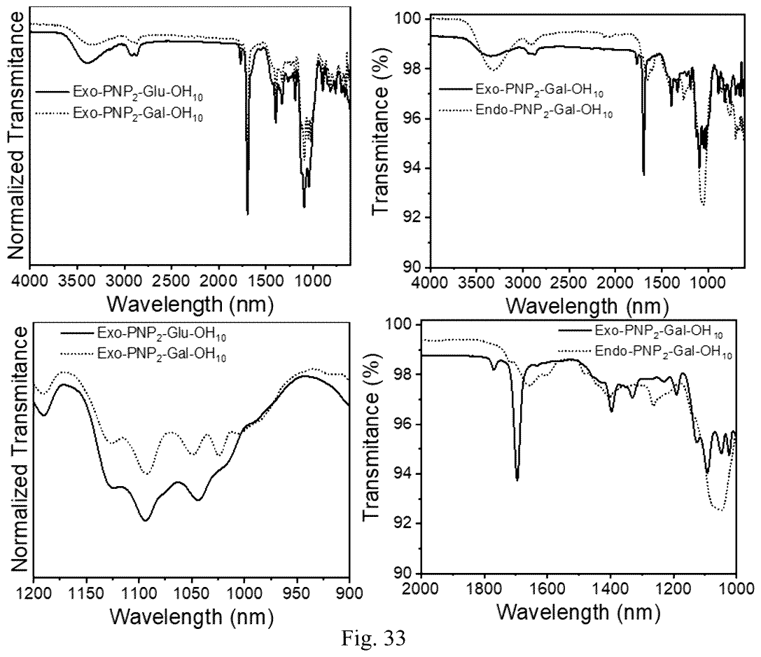

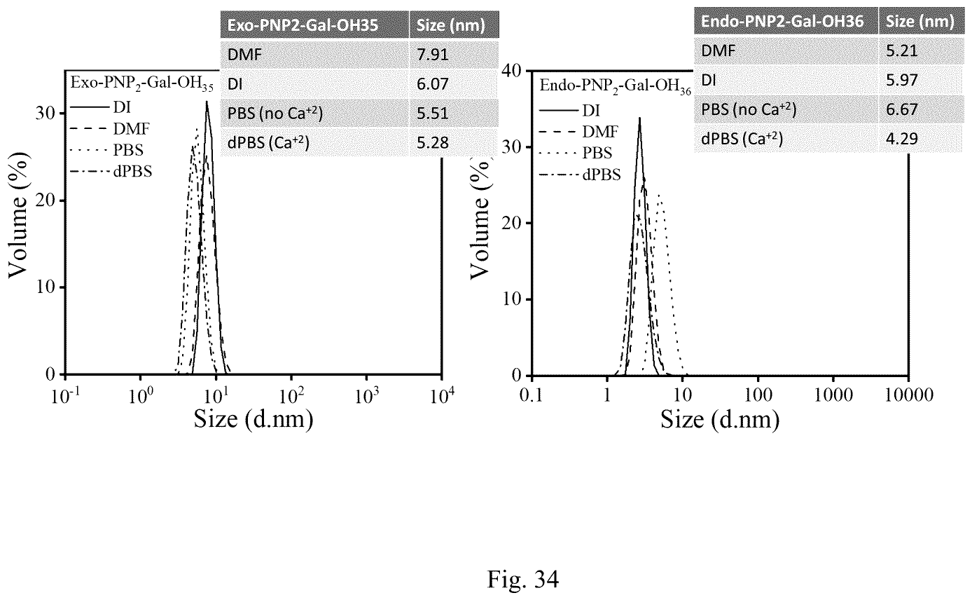

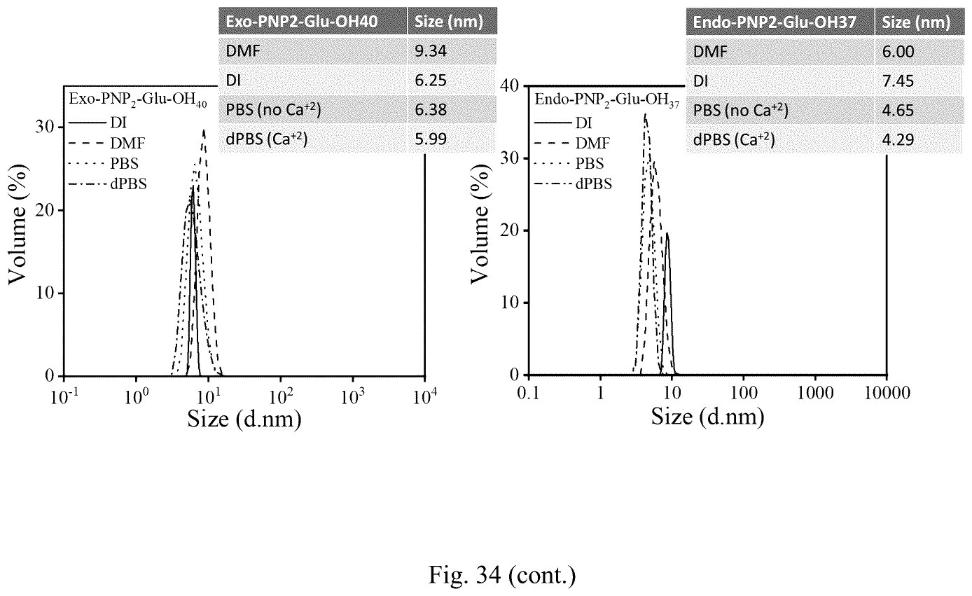

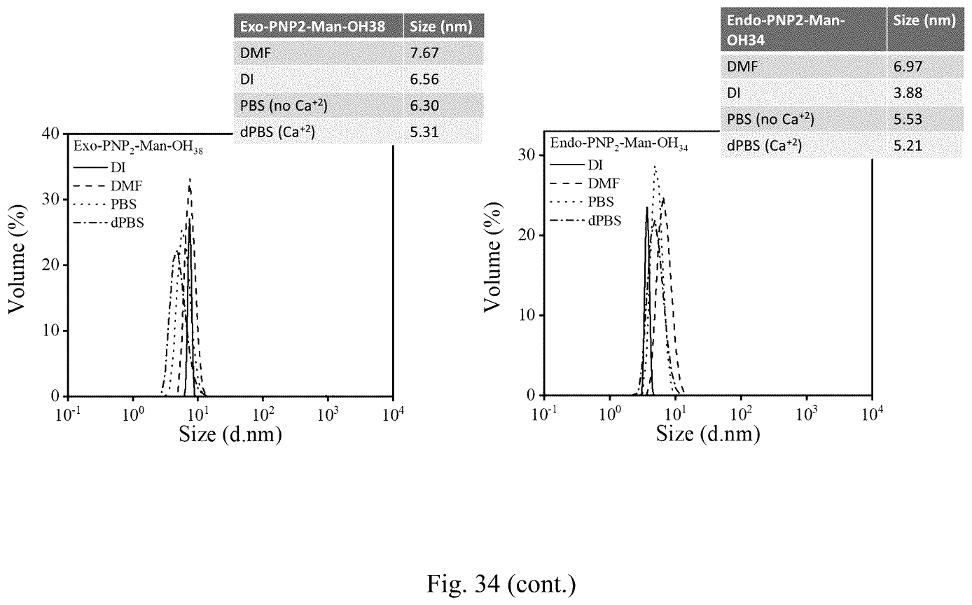

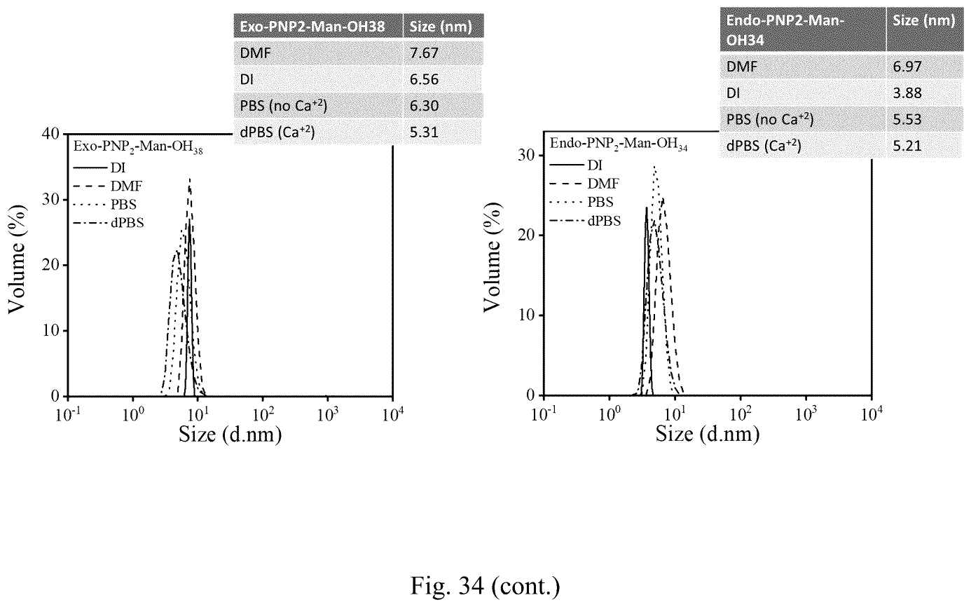

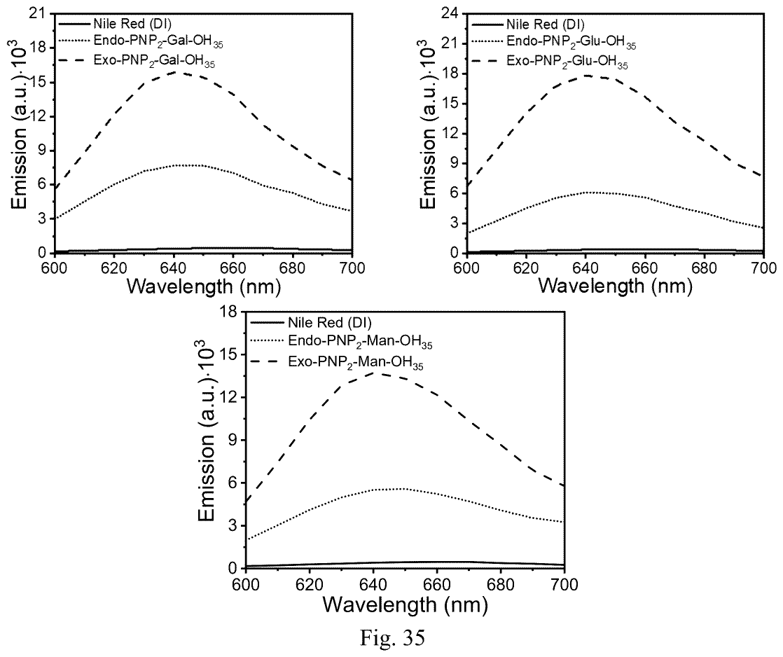

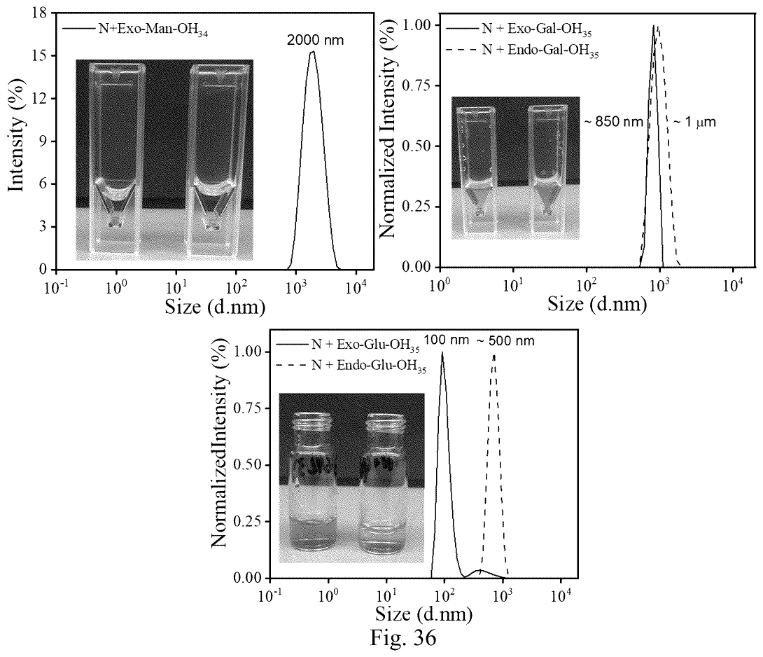

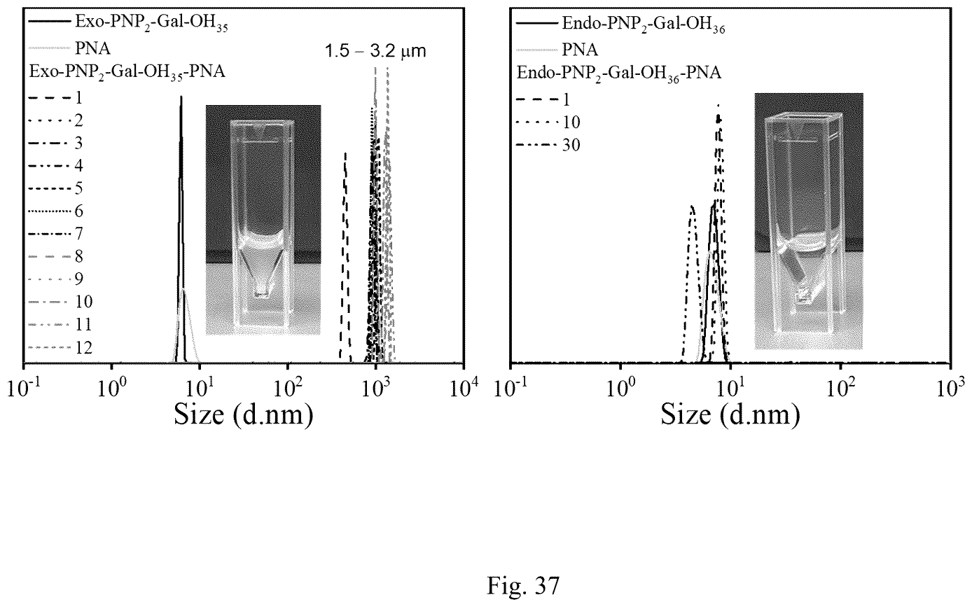

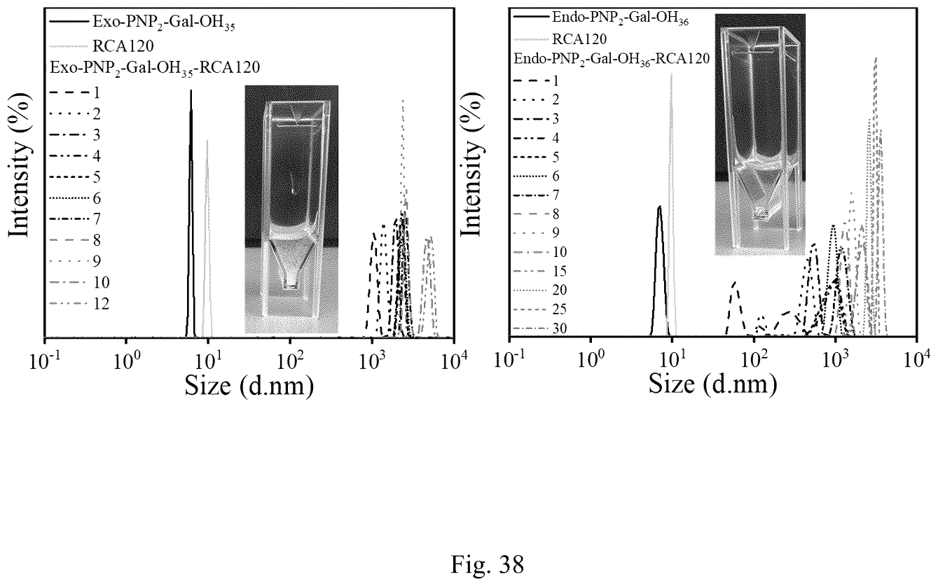

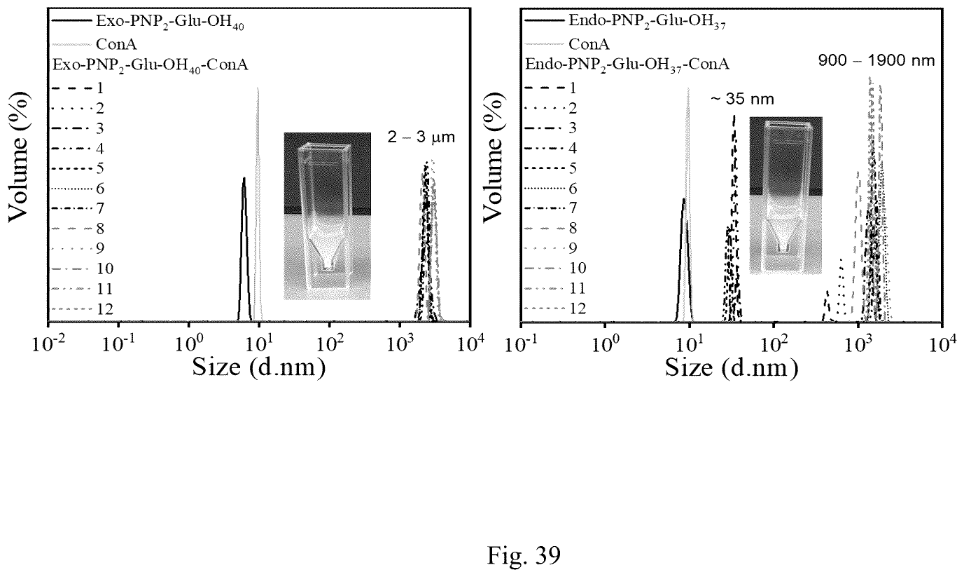

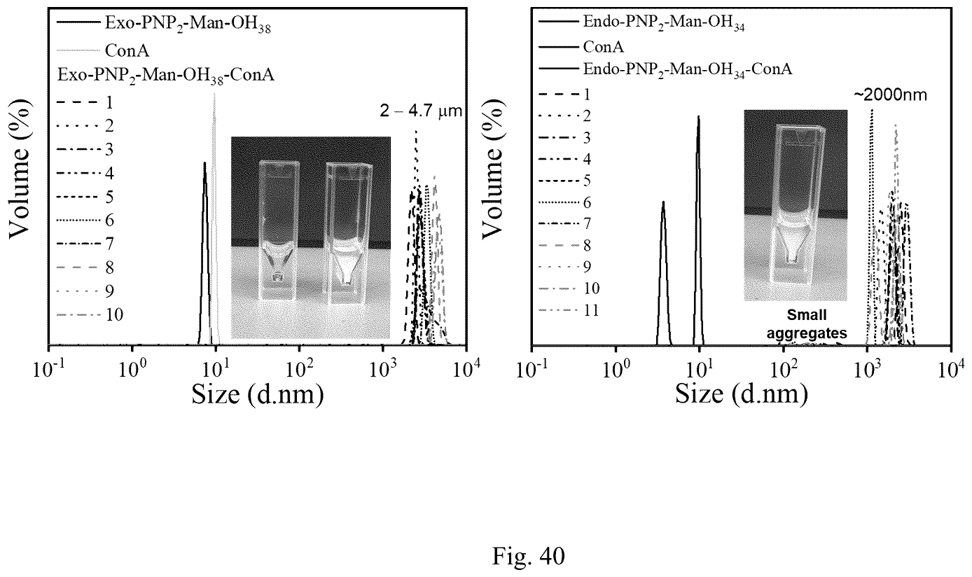

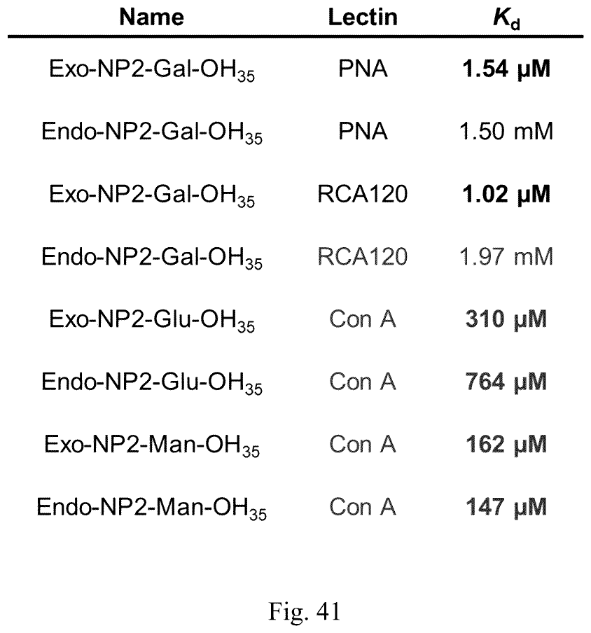

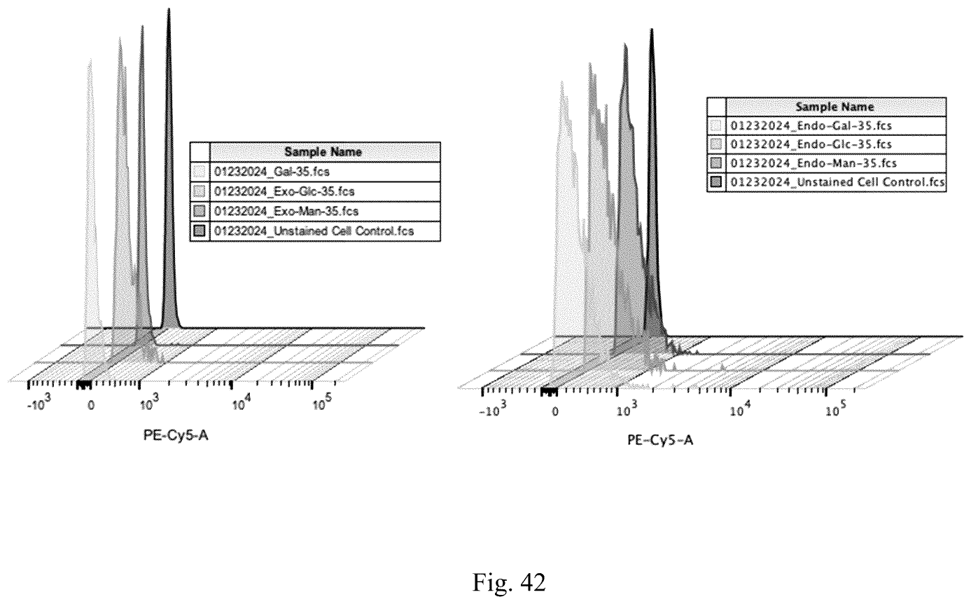

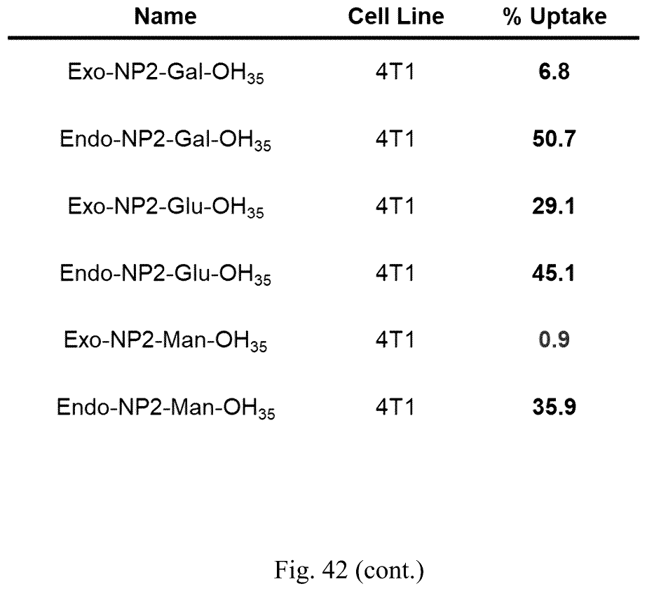

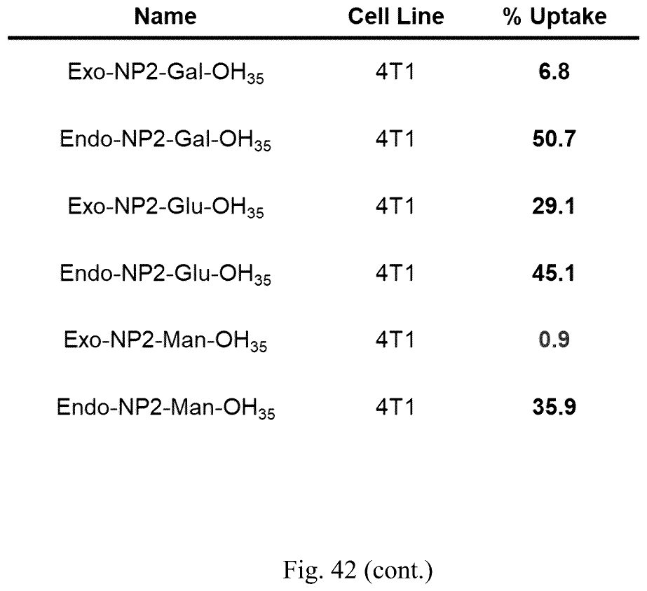

The nanoassemblies were imaged using transmission electron microscopy (TEM) (FIG. 32). Both Exo-Gal and Exo-Glc polymers showed hydrogen bonding interactions under IR (FIG. 33). Dynamic light scattering (DLS) data was used to determine size of the polymers as a function of solvent (FIG. 34). Exo polymers were found to more efficiently capture Nile red (hydrophobic dyes) and form micelles. Endo polymers captured significantly less dye, with less micelle formation (FIG. 35). The Endo and Exo polymers plus nile red and corresponding DLSs showed that exo polymers formed micelles with hydrophobic dyes (FIG. 36). Exo-Gal polymers also rapidly bind PNA, whereas Endo-Gal polymers show no evidence of protein binding (FIG. 37) and Exo-Gal polymers rapidly bind RCA120, whereas Endo-Gal polymers bind RCA120 much slower (FIG. 38). Both exo- and endo-Glc bind ConA, but Endo-glc forms smaller aggregates with ConA at first, before forming larger aggregates like the exo (FIG. 39). Both mannose polymers rapidly bind ConA, with endo polymers first forming smaller aggregates, and then larger (FIG. 40). Polymer-lectin binding was also measured by microscale thermophoresis (FIG. 41) and the cell uptake of polymers as measured by flow cytometry showed that all polymers entered cells, but all endo polymers entered cells to a higher extent than their exo counterparts (FIG. 42).

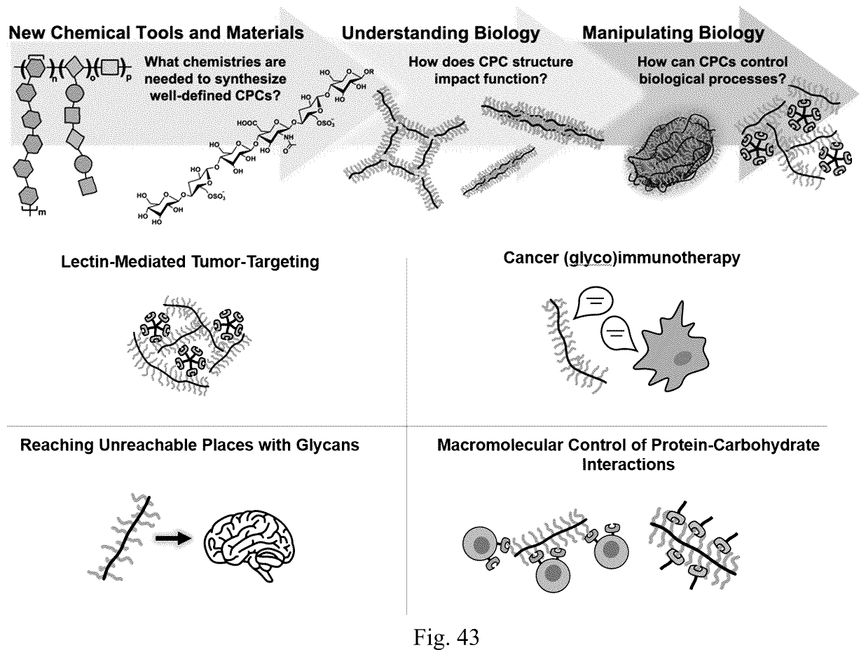

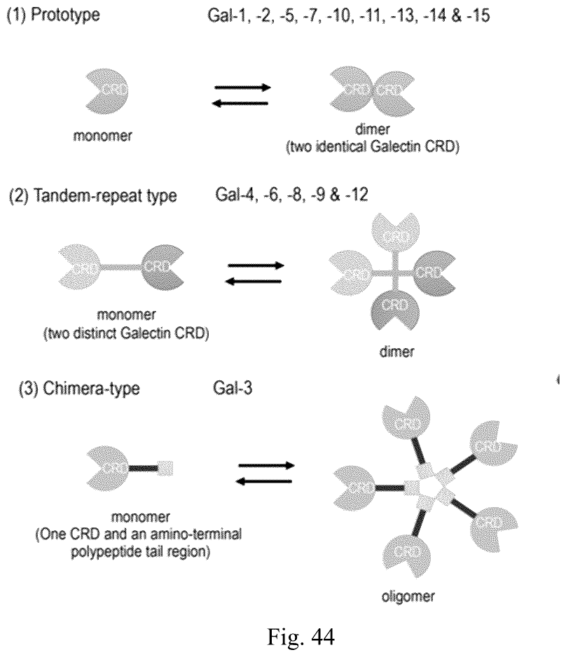

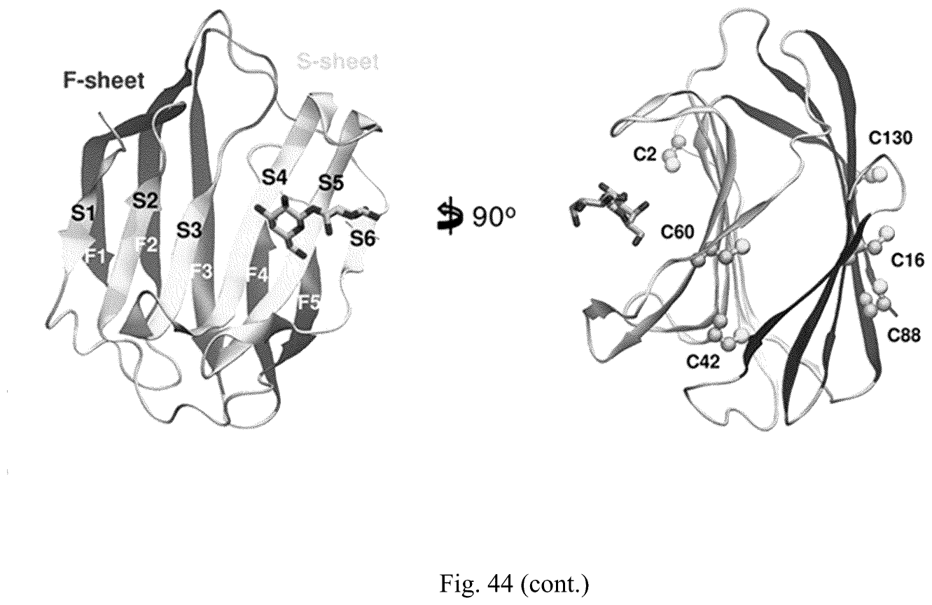

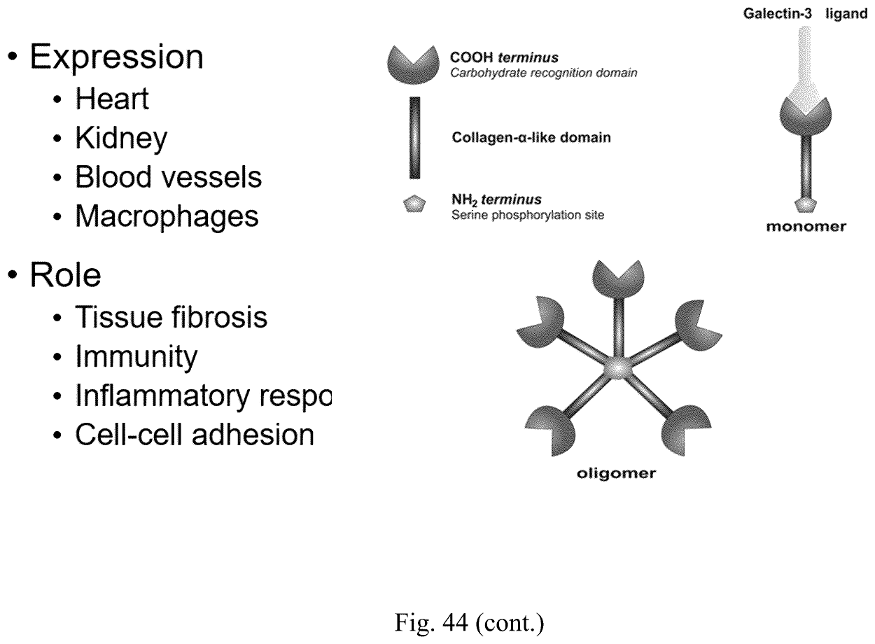

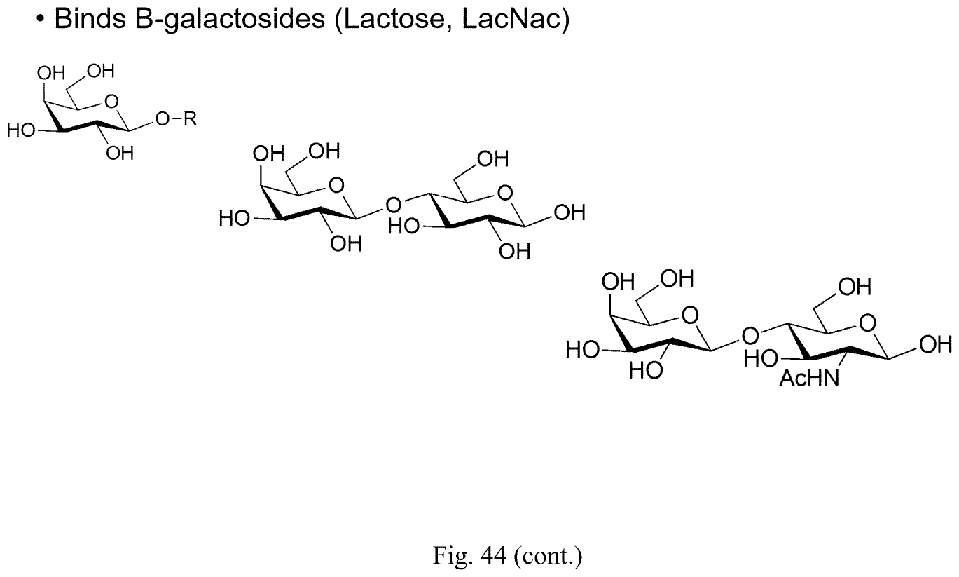

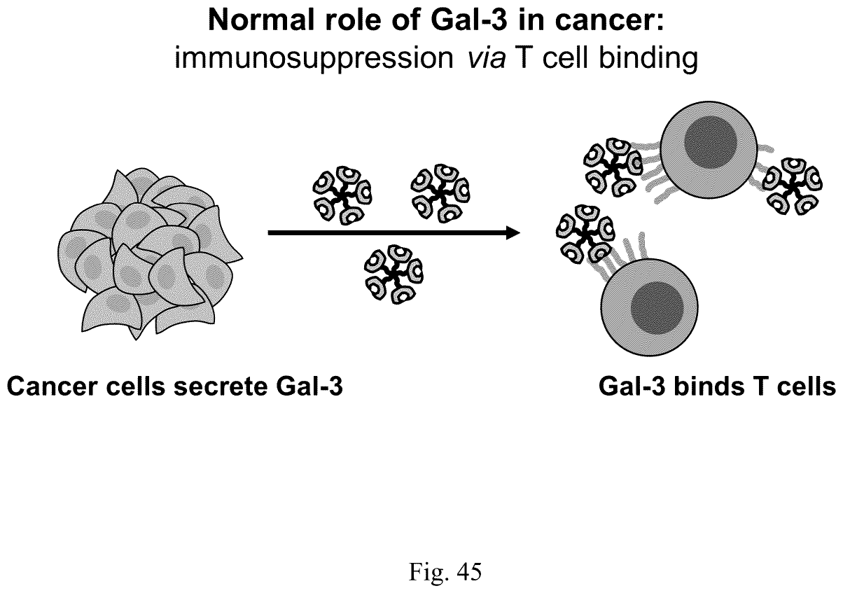

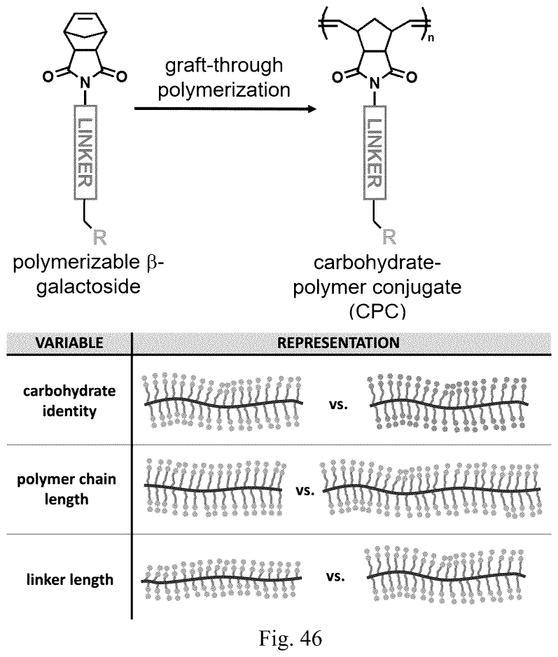

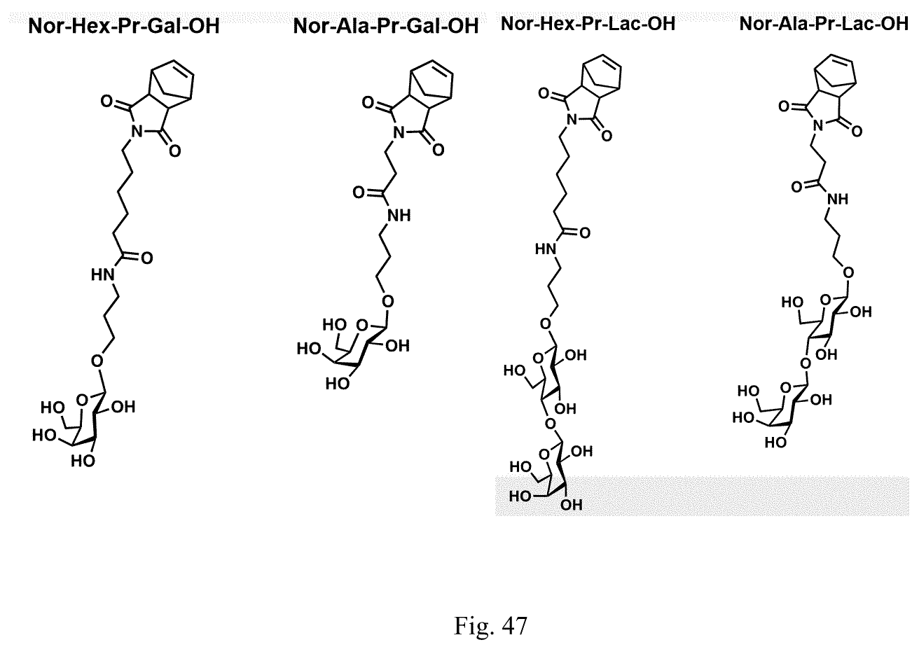

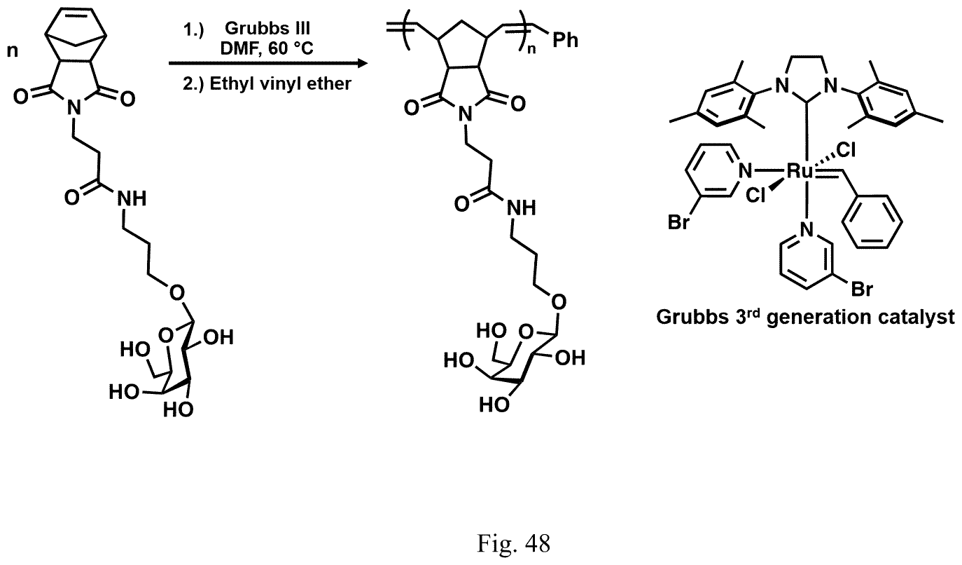

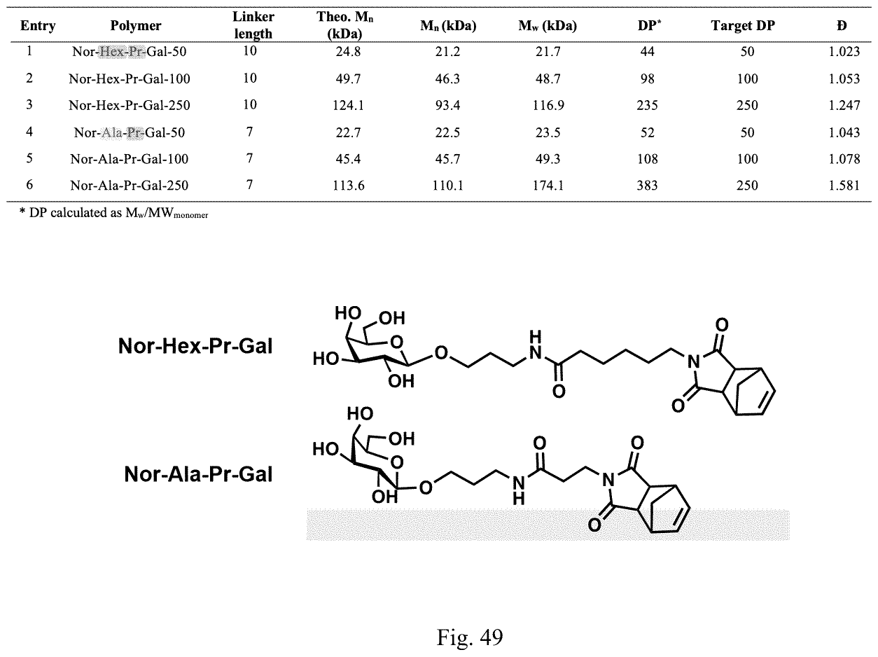

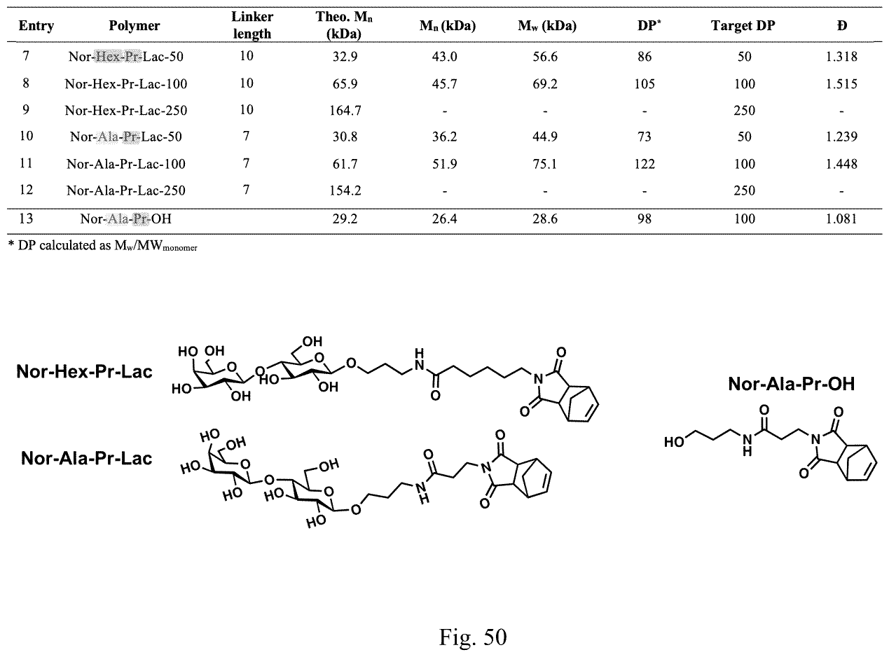

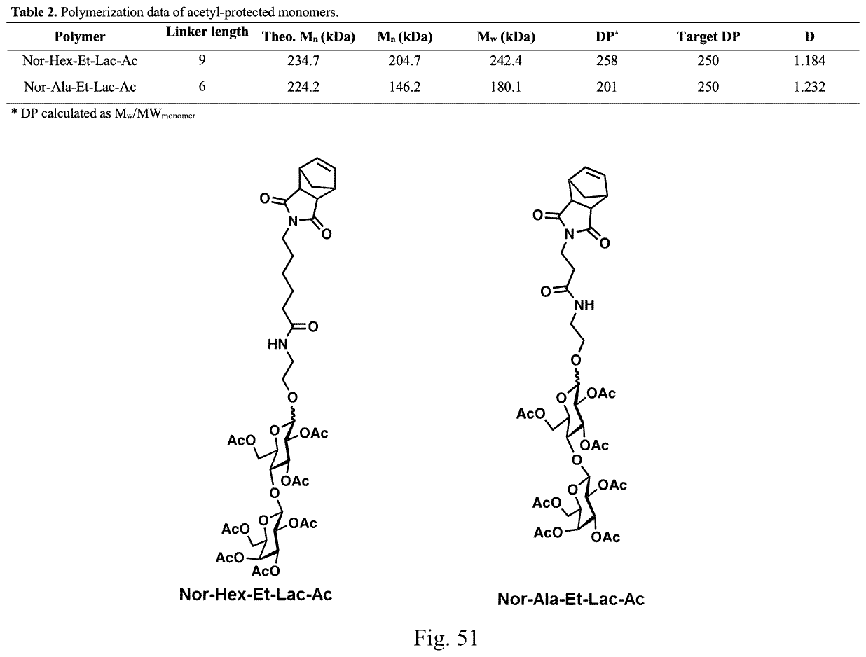

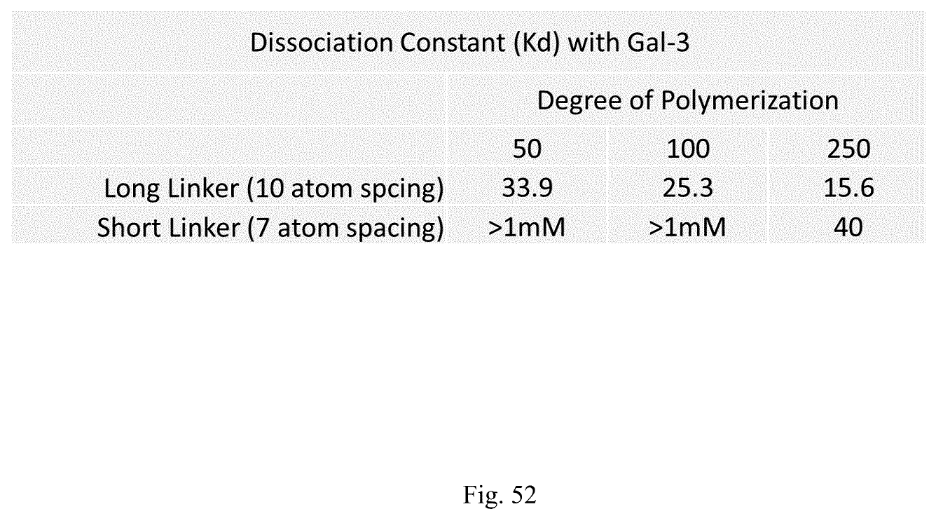

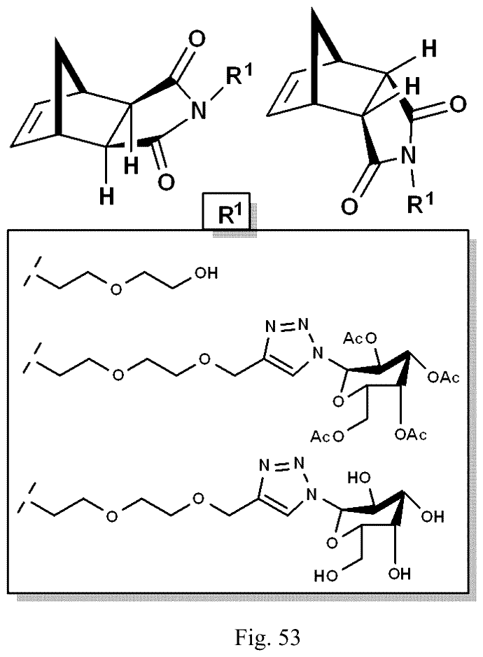

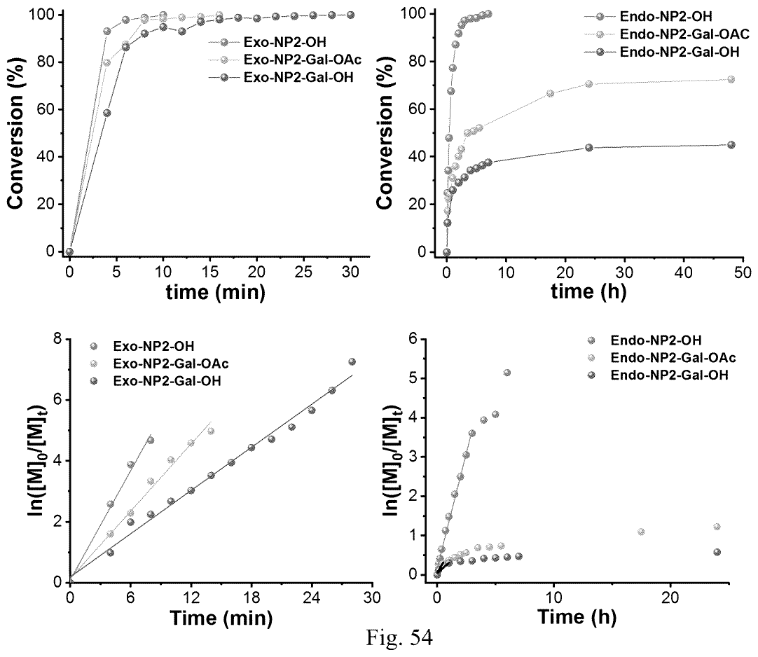

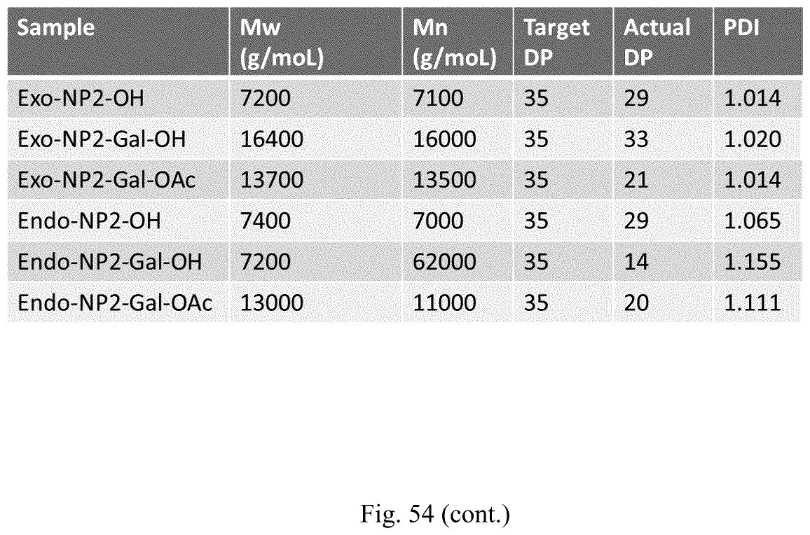

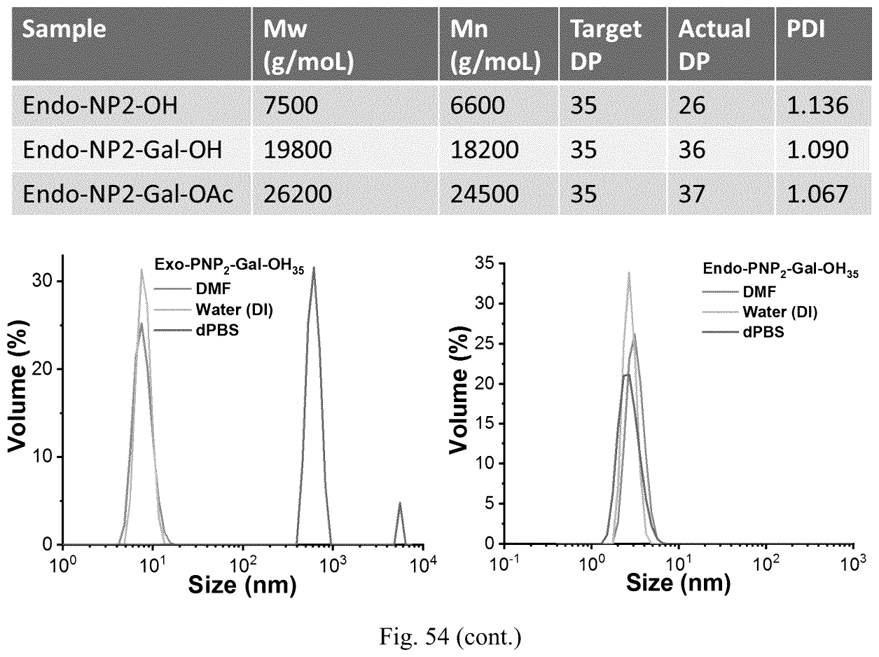

In general, the present invention works to leverage the power of carbohydrates and synthetic polymer chemistry to understand and manipulate biology with soft materials (FIG. 43). Galactin-3 was selected as a target as it is upregulated in cancer and other inflammatory diseases (FIG. 44) and plays a role in anti-cancer immunity (FIG. 45). The ultimate goal of the present invention is to develop CPCs that bind Gal-3 with high avidity and specificity with the hypothesis that the macromolecular structure and presentation of carbohydrates dictates the avidity of galectin-3 binding (FIG. 46). Representative monomers (FIG. 47) are subjected to ROMP (FIG. 48, FIG. 49, FIG. 50, FIG. 51). In these representative studies, Galactose-CPCs were found to bind Gal-3 with micromolar affinity (FIG. 52). Derivatives of monomers were further synthesized (FIG. 53) and analysis of the resultant polymer showed differences in behavior between Endo-CPCs and Exo-CPCs (FIG. 54).

### Example 3: Synthesis of Glycomonomers and Experimental Procedures

All materials and reagents, unless otherwise noted, were purchased from either Sigma Aldrich or Fisher Chemicals. cis-5-Norbornene-exo-2,3-dicarboxylic anhydride was purchased from Oakwood Chemical. (+)-camphor-10-sulfonic acid (CSA) was purchased from TCI chemicals. Benzoyl chloride (BzCl) was purchased from Beantown Chemical. N-iodosuccinimide (NIS), 1,4-dithio-DL-threitol (DTT) were purchased from VWR. Ethyl 1-thio-β-D-glucopyranoside and 4-aminobutan-1-ol were purchased from Ambeed. Dry solvents, dimethylformamide (DMF), dichloromethane (DCM), and methanol (MeOH) were prepared by drying via VAC solvent purification system (SPS), distillation, and sieves respectively.

Nuclear magnetic resonance spectra were recorded at ambient temperature unless stated otherwise with an Agilent MR 400 (400 MHz), Varian MR-400 (400 MHz), Bruker NEO400 (400 MHz), Bruker AVIII 3M 500 (500 MHz), or Bruker Avance III HD fitted with a Prodigy cryoprobe (500 MHz). Solvent resonances were referenced for ¹H and ¹³C NMR chemical shifts and reported in ppm. Multiplicity data reported as s=singlet, d=doublet, t=triplet, q=quartet, p=pentet, and m=multiplet. High resolution ESI-MS was obtained using an Agilent Technologies 6530 Accurate-Mass Q-TOF. Polymer characterization was completed using a Tosoh Bioscience EcoSEC Elite HLC-8420GPC fitted with a TSKgel Alpha-M (0018344) column and a Wyatt DAWN 8 (WD3-03) light scattering detector. The GPC-LS system was run in 10 mM LiBr in DMF at 60° C. Dynamic light scattering (DLS) measurements were taken using a Zetasizer Nano ZS (Dispersant: H2O RI=1.330, Viscosity=0.8872 or 1×PBS RI=1.332, Viscosity=0.9043; Material: RI=1.45, Absorption=0.001; T=25° C.) with three runs per measurement, 10 seconds per run, and at least 3 measurements averaged together.

All cell lines were obtained from ATCC or Sigma Aldrich. Cells were incubated at 37° C. at 5% CO₂ using RPMI-1640 (Fisher Scientific, Cat: 11875093) supplemented with 10% fetal bovine serum (Fisher Scientific, Cat: 35010CV), and 1% penicillin/streptomycin (Fisher Scientific, Cat: SV30010). Cells cultures were maintained by subculturing in flasks every 2-5 days as necessary per cell growth using trypsin-EDTA, 0.05% (Fisher Scientific, Cat: MT25052Cl).

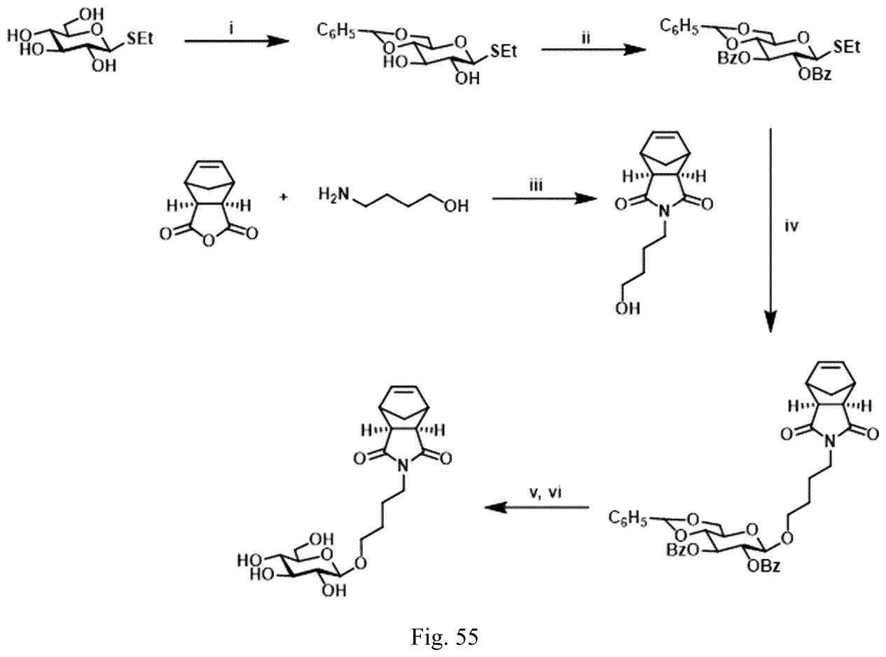

General synthetic pathways are shown in FIG. 55.

### 6-((3aR,7aS)-1,3-dioxo-1,3,3a,4,7,7a-hexahydro-2H-4, 7-methanoisoindol-2-yl)hexanoic acid: Nor-Hex (1)

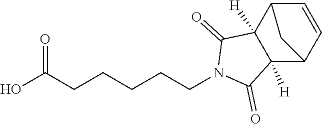 [SMILES: O=C(O)CCCCCN1C(=O)[C@@H]2C3C=CC(C3)[C@@H]2C1=O]

Carboxylic acid Xa was prepared using a previously established method (Patel et al., 2012, Biomacromolecules, 13, 2546).

¹H NMR (400 MHz, CDCl₃) δ: 6.27 (s, 2H), 3.45 (t, J=7.4 Hz, 2H), 3.26 (s, 2H), 2.66 (s, 2H), 2.33 (t, J=7.4 Hz, 2H), 1.73-1.52 (m, 4H), 1.50 (d, J=9.9 Hz, 1H), 1.34 (p, J=8.0 Hz, 2H), 1.20 (d, J=9.8 Hz, 1H).

### 3-((3aR,7aS)-1,3-dioxo-1,3,3a,4,7,7a-hexahydro-2H-4, 7-methanoisoindol-2-yl)propanoic acid: Nor-Ala (2)

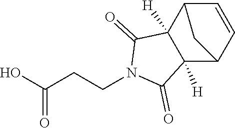 [SMILES: O=C(O)CCN1C(=O)[C@@H]2C3C=CC(C3)[C@@H]2C1=O]

Carboxylic acid Xa was prepared using a previously established method (Zhang et al., 2013, J. Am. Chem. Soc., 135, 15994).

¹H NMR (500 MHz, CDCl₃) δ: 6.29 (s, 2H), 3.79 (t, J=7.3 Hz, 2H), 3.28 (s, 2H), 2.74-2.61 (m, 4H), 1.52 (d, J=9.9 Hz, 1H), 1.25 (d, J=9.9 Hz, 1H).

### 3-((tert-butyldimethylsilyl)oxy)propan-1-amine (3)

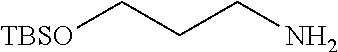 [SMILES: CC(C)(C)[Si](C)(C)OCCCN]

TBS protected alcohol Xa was prepared using a previously established method and used without further purification³.

¹H NMR (500 MHz, CDCl₃) δ: 3.64 (t, J=6.0 Hz, 2H), 2.74 (t, J=6.8 Hz, 2H), 1.72 (s, 2H), 1.60 (p, J=6.4 Hz, 2H), 0.83 (s, 9H), −0.01 (s, 6H).

### N-(3-((tert-butyldimethylsilyl)oxy)propyl)-6-((3aR,7aS)-1,3-dioxo-1,3,3a,4,7,7a-hexahydro-2H-4, 7-methanoisoindol-2-yl)hexanamide: Nor-Hex-Pr-TBS (4)

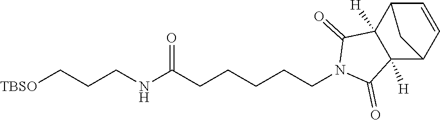 [SMILES: CC(C)(C)[Si](C)(C)OCCCNC(=O)CCCCCN1C(=O)[C@@H]2C3C=CC(C3)[C@@H]2C1=O]

Carboxylic acid 1 (3.6 mmol, 1 eq.), 3-((tert-butyldimethylsilyl)oxy)propan-1-amine (5.4 mmol, 1.5 eq.), and DMAP (1.44 mmol, 0.4 eq.) were dissolved in dry DCM (10 mL). Upon cooling to 0° C., DCC (7.2 mmol, 2 eq.) dissolved in dry DCM (5 mL) was dripped into the reaction mixture. The ice bath was allowed to expire, and the reaction was stirred at room temperature overnight. The reaction was filtered to remove the precipitated dicyclohexyl urea (DCU) and the filter cake washed with DCM. The reaction mixture was subjected to an aqueous workup consisting of a wash with water followed by drying over Na₂SO₄ and concentration under vacuum. Flash column chromatography was conducted (SiO₂: 5% to 50% EtOAc in hexanes) and the title compound 4 was isolated as a yellow oil in 71% yield (1153.17 mg, 2.57 mmol).

¹H NMR (500 MHz, CDCl₃) δ: 6.28 (s, 2H), 6.15 (s, 1H), 3.73 (t, J=5.6 Hz, 2H), 3.44 (t, J=7.5 Hz, 2H), 3.36 (q, J=5.9 Hz, 2H), 3.26 (s, 2H), 2.66 (s, 2H), 2.11 (t, J=7.6 Hz, 2H), 1.70 (p, J=5.9 Hz, 2H), 1.64 (p, J=7.7 Hz, 2H), 1.56 (q, J=7.6 Hz, 2H), 1.50 (d, J=9.9 Hz, 1H), 1.31 (p, J=8.0 Hz, 2H), 1.20 (d, J=9.8 Hz, 1H), 0.90 (s, 9H), 0.06 (s, 6H).

¹³C NMR (126 MHz, CDCl₃) δ: 178.20, 172.56, 137.95, 62.84, 47.92, 45.27, 42.86, 38.61, 38.58, 36.74, 31.57, 27.64, 26.77, 26.05, 25.41, 18.35, −5.30.

HRMS for C₂₄H₄₀N₂O₄Si: [M+Na]⁺ calc.=471.2650; found=471.2658.

### N-(3-((tert-butyldimethylsilyl)oxy)propyl)-3-((3aR,7aS)-1,3-dioxo-1,3,3a,4,7,7a-hexahydro-2H-4,7-methanoisoindol-2-yl)propanamide: Nor-Ala-Pr-TBS (5)

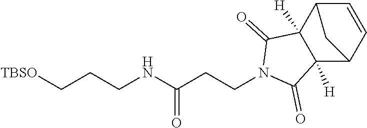 [SMILES: CC(C)(C)[Si](C)(C)OCCCNC(=O)CCN1C(=O)[C@@H]2C3C=CC(C3)[C@@H]2C1=O]

Carboxylic acid 2 (3.6 mmol, 1 eq.), 3-((tert-butyldimethylsilyl)oxy)propan-1-amine (5.4 mmol, 1.5 eq.), and DMAP (1.44 mmol, 0.4 eq.) were dissolved in dry DCM (10 mL). Upon cooling to 0° C., DCC (7.2 mmol, 2 eq.) dissolved in dry DCM (5 mL) was dripped into the reaction mixture. The ice bath was allowed to expire, and the reaction was stirred at room temperature overnight. The reaction was filtered to remove the precipitated dicyclohexyl urea (DCU) and the filter cake washed with DCM. The reaction mixture was subjected to an aqueous workup consisting of a wash with water followed by drying over Na₂SO₄ and concentration under vacuum. Flash column chromatography was conducted (SiO₂: 5% to 50% EtOAc in hexanes) and the title compound 5 was isolated as a yellow oil in 77% yield (1122.39 mg, 2.76 mmol).

¹H NMR (500 MHz, CDCl₃) δ: 6.28 (s, 2H), 6.25 (s, 1H), 3.78 (t, J=7.3 Hz, 2H), 3.72 (t, J=5.6 Hz, 2H), 3.34 (q, J=6.0 Hz, 2H), 3.27 (s, 2H), 2.68 (s, 2H), 2.46 (t, J=7.3 Hz, 2H), 1.69 (p, J=5.9 Hz, 2H), 1.50 (d, J=9.9 Hz, 1H), 1.24 (d, J=9.6 Hz, 1H), 0.89 (s, 9H), 0.06 (s, 6H).

¹³C NMR (126 MHz, CDCl₃) δ: 177.97, 169.32, 137.98, 62.60, 47.95, 45.34, 42.87, 38.55, 35.17, 33.98, 31.49, 26.05, 18.34, −5.30.

HRMS for C₂₁H₃₄N₂O₄Si: [M+Na]⁺ calc.=429.2180; found=429.2191.

### 6-((3aR,7aS)-1,3-dioxo-1,3,3a,4,7,7a-hexahydro-2H-4, 7-methanoisoindol-2-yl)-N-(3-hydroxypropyl)hexanamide: Nor-Hex-Pr-OH (6)

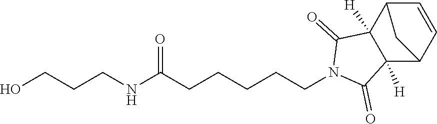 [SMILES: O=C(CCCCCN1C(=O)[C@@H]2C3C=CC(C3)[C@@H]2C1=O)NCCCO]

Silyl-protected alcohol 4 (2.57 mmol, 1 eq.) was dissolved in a minimal amount of anhydrous methanol. Acetyl chloride (0.39 mmol, 0.15 eq.) was added to reaction and stirred at room temperature overnight. The reaction mixture was concentrated under reduced pressure.

Flash column chromatography was conducted (SiO₂: 80% to 100% EtOAc in hexanes) and the title compound 6 was isolated as a yellow oil in 91% yield (777.95 mg, 2.33 mmol).

¹H NMR (400 MHz, MeOD) δ 6.32 (s, 2H), 3.58 (t, J=6.3 Hz, 2H), 3.45 (t, J=7.2 Hz, 2H), 3.25 (t, J=7.0 Hz, 2H), 3.18 (t, J=1.9 Hz, 2H), 2.71 (s, 2H), 2.17 (t, J=7.4 Hz, 2H), 1.76-1.45 (m, 7H), 1.37-1.18 (m, 3H).

¹³C NMR (101 MHz, MeOD) δ 180.09, 176.09, 138.90, 60.41, 46.31, 43.49, 39.35, 37.34, 36.80, 33.25, 28.46, 27.55, 26.48.

HRMS for C₁₈H₂₆N₂O₄: [M+Na]⁺ calc.=357.1785; found=357.1787.

### 3-((3aR,7aS)-1,3-dioxo-1,3,3a,4,7,7a-hexahydro-2H-4, 7-methanoisoindol-2-yl)-N-(3-hydroxypropyl)propanamide: mono-OH (7)

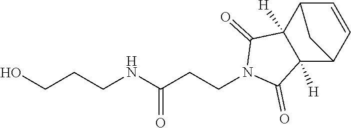 [SMILES: O=C(CCN1C(=O)[C@@H]2C3C=CC(C3)[C@@H]2C1=O)NCCCO]

Silyl-protected alcohol 5 (2.76 mmol, 1 eq.) was dissolved in a minimal amount of anhydrous methanol. Acetyl chloride (0.41 mmol, 0.15 eq.) was added to reaction and stirred at room temperature overnight. The reaction mixture was concentrated under reduced pressure. Flash column chromatography was conducted (SiO₂: 80% to 100% EtOAc in hexanes) and the title compound 7 was isolated as a white solid in 67% yield (539.74 mg, 1.85 mmol).

¹H NMR (400 MHz, MeOD) δ: 6.32 (s, 2H), 3.73 (t, J=7.0 Hz, 2H), 3.57 (t, J=6.3 Hz, 2H), 3.21 (t, J=7.0 Hz, 2H), 3.18 (s, 2H), 2.70 (s, 2H), 2.46 (t, J=7.0 Hz, 2H), 1.68 (p, J=6.6 Hz, 2H), 1.47 (d, J=9.8 Hz, 1H), 1.30 (d, J=9.7 Hz, 1H).

¹³C NMR (101 MHz, MeOD) δ: 179.65, 138.93, 60.40, 49.03, 46.30, 43.59, 37.43, 36.33, 34.69, 33.09.

HRMS for C₁₅H₂₀N₂O₄: [M+Na]⁺ calc.=315.1315; found=315.1321.

### 6-((3aR,7aS)-1,3-dioxo-1,3,3a,4,7,7a-hexahydro-2H-4, 7-methanoisoindol-2-yl)-N-(3-(((2R,3R,4S,5R,6R)-3,4,5-trihydroxy-6-(hydroxymethyl)tetrahydro-2H-pyran-2-yl)oxy)propyl)hexanamide: mono-Gal₁₀ (8)

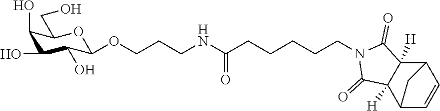

Alcohol 6 (0.51 mmol, 1 eq.) and per-acetylated galactose (0.61 mmol, 1.2 eq.) were combined and placed under high-vac overnight. Under N₂, a minimal volume of dry DCM was added to the reaction vessel. Following dissolution, the reaction was cooled to 0° C. and BF₃·Et₂O (1.53 mmol, 3 eq.) was dripped into the reaction. The reaction was stirred at room temperature for 4 hours followed by another addition of BF₃·Et₂O (1.53 mmol, 3 eq.). The reaction was stirred overnight and then quenched via the addition of saturated NaHCO₃. After the reaction was stirred with saturated NaHCO₃ for ten minutes, the mixture was transferred to a separatory funnel. The mixture was separated, and the aqueous layer extracted with DCM (×3). The organic fractions were combined together and washed with dilute brine, dried over Na₂SO₄, and then concentrated under reduced pressure. Flash column chromatography was conducted (SiO₂: 80% to 100% EtOAc in hexanes) to isolate the per-acetylated and per-acetylated minus 1 acetyl-norbornene-galactose conjugates in an 82% crude yield.

The aforementioned galactose conjugates were then subjected to base promoted acetyl deprotection. The compounds were dissolved in a minimal amount of MeOH followed by the addition of NaOMe (0.1 mmol, 0.2 eq.). The reaction was monitored via TLC and sequential additions of NaOMe (0.1 mmol, 0.2 eq.) were added until reaction completion. Upon completion, the reaction was quenched with Dowex-X8 H⁺ resin until the pH reached 6-7.5. The mixture was rapidly filtered, resin washed with MeOH, and then concentrated under reduced pressure. The crude product was then subjected to flash column chromatography (SiO₂: 100% acetone) and the title compound 8 was isolated as a white solid in 37% yield over two steps (77.7 mg, 0.16 mmol).

¹H NMR (400 MHz, MeOD) δ: 6.32 (s, 2H), 4.23 (d, J=7.2 Hz, 1H), 3.93 (m, 1H), 3.83 (d, J=3.1 Hz, 1H), 3.80-3.68 (m, 2H), 3.62 (m, 1H), 3.55-3.42 (m, 6H), 3.18 (s, 2H), 2.72 (s, 2H), 2.21-2.14 (m, 3H), 1.79 (p, J=6.3 Hz, 2H), 1.68-1.53 (m, 4H), 1.49 (d, J=10.1 Hz, 1H), 1.31 (m, 3H), 1.21 (d, J=9.8 Hz, 1H).

¹³C NMR (101 MHz, MeOD) δ 180.21, 176.04, 138.89, 104.95, 76.68, 75.00, 72.58, 70.34, 68.35, 62.56, 46.31, 43.48, 39.39, 37.61, 36.84, 30.32, 28.45, 27.53, 26.48.

HRMS for C₂₄H₃₆N₂O₉: [M+H]⁺ calc.=497.2494; found=497.2502.

### N-(3-(((2R,3R,4R,5S,6R)-3,4-dihydroxy-6-(hydroxymethyl)-5-(((2S,3R,4S,5R,6R)-3,4,5-trihydroxy-6-(hydroxymethyl)tetrahydro-2H-pyran-2-yl)oxy)tetrahydro-2H-pyran-2-yl)oxy)propyl)-6-((3aR,7aS)-1,3-dioxo-1,3,3a,4,7,7a-hexahydro-2H-4, 7-methanoisoindol-2-yl)hexanamide: mono-Lac₁₀ (9)

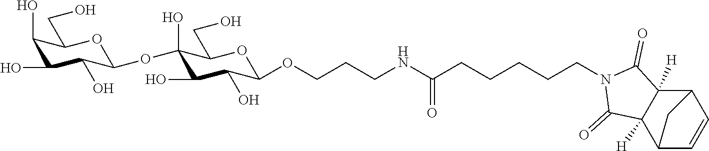

Alcohol 6 (0.29 mmol, 1 eq.) and per-acetylated lactose (0.35 mmol, 1.2 eq.) were combined and placed under high-vac overnight. Under N₂, a minimal volume of dry DCM was added to the reaction vessel. Following dissolution, the reaction was cooled to 0° C. and BF₃·Et₂O (0.87 mmol, 3 eq.) was dripped into the reaction. The reaction was stirred at room temperature for 4 hours followed by another addition of BF₃·Et₂O (0.87 mmol, 3 eq.). The reaction was stirred overnight and then quenched via the addition of saturated NaHCO₃. After the reaction was stirred with saturated NaHCO₃ for ten minutes, the mixture was transferred to a separatory funnel. The mixture was separated, and the aqueous layer extracted with DCM (×3). The organic fractions were combined together and washed with dilute brine, dried over Na₂SO₄, and then concentrated under reduced pressure. Flash column chromatography was conducted (SiO₂: 80% to 100% EtOAc in hexanes) to isolate the per-acetylated and per-acetylated minus 1 acetyl-norbornene-lactose conjugate with a 62% crude yield.

The aforementioned lactose conjugates were then subjected to base promoted acetyl deprotection. The compounds were dissolved in a minimal amount of MeOH followed by the addition of NaOMe (0.058 mmol, 0.2 eq.). The reaction was monitored via TLC and sequential additions of NaOMe (0.058 mmol, 0.2 eq.) were added until reaction completion. Upon completion, the reaction was quenched with Dowex-X8 H⁺ resin until the pH reached 6-7.5. The mixture was rapidly filtered, resin washed with MeOH, and then concentrated under reduced pressure. The crude product was then subjected to flash column chromatography (SiO₂: 0% to 10% MeOH in acetone) and the title compound 9 was isolated as a white solid in 40% yield over two steps (78.2 mg, 0.12 mmol).

¹H NMR (400 MHz, MeOD) δ: 6.33 (s, 2H), 4.36 (d, J=7.5 Hz, 1H), 4.31 (d, J=7.8 Hz, 1H), 3.98-3.66 (m, 6H), 3.66-3.38 (m, 10H), 3.30-3.22 (m, 2H), 3.18 (s, 2H), 2.71 (s, 2H), 2.17 (t, J=7.4 Hz, 2H), 1.79 (p, J=6.4 Hz, 2H), 1.67-1.53 (m, 4H), 1.53-1.45 (m, 1H), 1.31 (p, J=8.2 Hz, 2H), 1.22 (d, J=9.7 Hz, 1H).

¹³C NMR (101 MHz, MeOD) δ: 180.14, 176.00, 138.90, 105.10, 104.16, 80.66, 77.09, 76.46, 74.84, 74.77, 72.56, 70.30, 68.44, 62.49, 61.93, 46.31, 43.49, 39.38, 37.62, 36.83, 30.32, 28.46, 27.53, 26.48.

HRMS for C₃₀H₄₆N₂O₁₄: [M+Na]⁺ calc.=681.2841; found=681.2857.

### 3-((3aR,7aS)-1,3-dioxo-1,3,3a,4,7,7a-hexahydro-2H-4, 7-methanoisoindol-2-yl)-N-(3-(((2R,3R,4S,5R,6R)-3,4,5-trihydroxy-6-(hydroxymethyl)tetrahydro-2H-pyran-2-yl)oxy)propyl)propanamide: mono-Gal₇ (10)

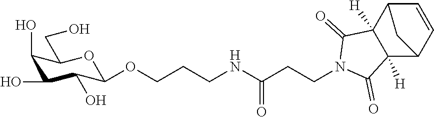

Alcohol 7 (0.51 mmol, 1 eq.) and per-acetylated galactose (0.61 mmol, 1.2 eq.) were combined and placed under high-vac overnight. Under N₂, a minimal volume of dry DCM was added to the reaction vessel. Following dissolution, the reaction was cooled to 0° C. and BF₃·Et₂O (1.53 mmol, 3 eq.) was dripped into the reaction. The reaction was stirred at room temperature for 4 hours followed by another addition of BF₃·Et₂O (1.53 mmol, 3 eq.). The reaction was stirred overnight and then quenched via the addition of saturated NaHCO₃. After the reaction was stirred with saturated NaHCO₃ for ten minutes, the mixture was transferred to a separatory funnel. The mixture was separated, and the aqueous layer extracted with DCM (×3). The organic fractions were combined together and washed with dilute brine, dried over Na₂SO₄, and then concentrated under reduced pressure. Flash column chromatography was conducted (SiO₂: 80% to 100% EtOAc in hexanes) to isolate the per-acetylated and per-acetylated minus 1 acetyl-norbornene-galactose conjugates in 79% crude yield.

The aforementioned galactose conjugates were then subjected to base promoted acetyl deprotection. The compounds were dissolved in a minimal amount of MeOH followed by the addition of NaOMe (0.1 mmol, 0.2 eq.). The reaction was monitored via TLC and sequential additions of NaOMe (0.1 mmol, 0.2 eq.) were added until reaction completion. Upon completion, the reaction was quenched with Dowex-X8 H⁺ resin until the pH reached 6-7.5. The mixture was rapidly filtered, resin washed with MeOH, and then concentrated under reduced pressure. The crude product was then subjected to flash column chromatography (SiO₂: 100% acetone) and the title compound 10 was isolated as a white solid in 32% yield over two steps (74.6 mg, 0.16 mmol).

¹H NMR (400 MHz, MeOD) δ: 6.32 (s, 2H), 4.23 (d, J=7.2 Hz, 1H), 3.92 (m, 1H), 3.82 (d, J=3.1 Hz, 1H), 3.74 (m, 4H), 3.61 (m, 1H), 3.55-3.47 (m, 3H), 3.25 (m, 2H), 3.18 (s, 2H), 2.71 (s, 2H), 2.46 (t, J=7.0 Hz, 2H), 1.76 (p, J=6.3 Hz, 2H), 1.47 (d, J=9.9 Hz, 1H), 1.30 (d, J=9.7 Hz, 1H).

¹³C NMR (101 MHz, MeOD) δ: 179.82, 172.85, 138.93, 104.92, 76.70, 75.00, 72.57, 70.34, 68.35, 62.57, 49.05, 46.31, 43.60, 37.70, 36.41, 34.76, 30.16.

HRMS for C₂₁H₃₀N₂O₉: [M+H]⁺ calc.=455.2024; found=455.2031.

### N-(3-(((2R,3R,4R,5S,6R)-3,4-dihydroxy-6-(hydroxymethyl)-5-(((2S,3R,4S,5R,6R)-3,4,5-trihydroxy-6-(hydroxymethyl)tetrahydro-2H-pyran-2-yl)oxy)tetrahydro-2H-pyran-2-yl)oxy)propyl)-3-((3aR,7aS)-1,3-dioxo-1,3,3a,4,7,7a-hexahydro-2H-4, 7-methanoisoindol-2-yl)propanamide: mono-Lac₇ (11)

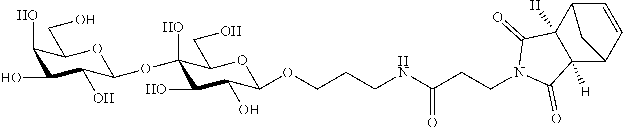

Alcohol 7 (0.29 mmol, 1 eq.) and per-acetylated lactose (0.35 mmol, 1.2 eq.) were combined and placed under high-vac overnight. Under N₂, a minimal volume of dry DCM was added to the reaction vessel. Following dissolution, the reaction was cooled to 0° C. and BF₃·Et₂O (0.87 mmol, 3 eq.) was dripped into the reaction. The reaction was stirred at room temperature for 4 hours followed by another addition of BF₃·Et₂O (0.87 mmol, 3 eq.). The reaction was stirred overnight and then quenched via the addition of saturated NaHCO₃. After the reaction was stirred with saturated NaHCO₃ for ten minutes, the mixture was transferred to a separatory funnel. The mixture was separated, and the aqueous layer extracted with DCM (×3). The organic fractions were combined together and washed with dilute brine, dried over Na₂SO₄, and then concentrated under reduced pressure. Flash column chromatography was conducted (SiO₂: 80% to 100% EtOAc in hexanes) to isolate the per-acetylated and per-acetylated minus 1 acetyl-norbornene-lactose conjugates in 58% crude yield.

The aforementioned lactose conjugates were then subjected to base promoted acetyl deprotection. The compounds were dissolved in a minimal amount of MeOH followed by the addition of NaOMe (0.058 mmol, 0.2 eq.). The reaction was monitored via TLC and sequential additions of NaOMe (0.058 mmol, 0.2 eq.) were added until reaction completion. Upon completion, the reaction was quenched with Dowex-X8 H⁺ resin until the pH reached 6-7.5. The mixture was rapidly filtered, resin washed with MeOH, and then concentrated under reduced pressure. The crude product was then subjected to flash column chromatography (SiO₂: 0% to 10% MeOH in acetone) and the title compound 11 was isolated as a white solid in 37% yield over two steps (67.3 mg, 0.11 mmol).

¹H NMR (400 MHz, MeOD) δ: 6.32 (s, 2H), 4.36 (d, J=7.5 Hz, 1H), 4.31 (d, J=7.8 Hz, 1H), 3.96-3.39 (m, 16H), 3.25 (t, J=8.5 Hz, 3H), 3.18 (s, 2H), 2.71 (s, 2H), 2.46 (t, J=7.0 Hz, 2H), 1.77 (p, J=6.3 Hz, 2H), 1.47 (d, J=9.9 Hz, 1H), 1.30 (d, J=9.8 Hz, 1H).

¹³C NMR (101 MHz, MeOD) δ: 179.74, 172.85, 138.94, 105.09, 104.15, 80.64, 77.09, 76.48, 74.84, 74.77, 72.56, 70.31, 68.45, 62.50, 61.93, 46.31, 43.62, 37.70, 36.39, 34.77, 30.19.

HRMS for C₂₇H₄₀N₂O₁₄: [M+Na]⁺ calc.=639.2372; found=639.2377.

### (4aR,6S,7R,8R,8aS)-6-(ethylthio)-2-phenylhexahydropyrano[3,2-d][1,3]dioxine-7,8-diol: (12)

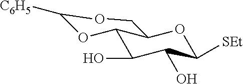

Benzaldehyde dimethyl acetal (10.0 mmol, 2 eq.) and ethyl 1-thio-β-D-glucopyranoside (5.0 mmol, 1 eq.) were dissolved in dry ACN (10 mL). CSA (0.5 mmol, 0.1 eq.) was added into the reaction mixture. The reaction was stirred at room temperature overnight. The reaction was quenched using TEA and subjected to an aqueous workup consisting of a wash with water followed by drying over Na₂SO₄ and concentration under vacuum. Flash column chromatography was conducted (SiO₂: 5% to 50% EtOAc in hexanes) and the title compound 12 was isolated as a white foam in 88% yield (1375.92 mg, 4.41 mmol).

¹H NMR (500 MHz, CDCl₃) δ: 7.52-7.45 (m, 2H), 7.41-7.33 (m, 3H), 5.55 (s, 1H), 4.47 (d, J=9.8 Hz, 1H), 4.36 (dd, J=10.5, 4.9 Hz, 1H), 3.84 (m, 1H), 3.77 (t, J=10.2 Hz, 1H), 3.58 (t, J=9.3 Hz, 1H), 3.55-3.47 (m, 2H), 2.81-2.73 (m, 3H), 2.60 (d, J=2.1 Hz, 1H), 1.33 (t, J=7.5 Hz, 3H).

¹³C NMR (126 MHz, CDCl₃) δ: 136.88, 129.32, 128.36, 126.27, 101.95, 86.66, 80.37, 74.58, 73.21, 70.58, 68.60, 24.80, 15.31.

HRMS for C₁₅H₂₀O₅S: [M+Na]⁺ calc.=335.9024; found=335.9032.

### (4aR,6S,7R,8S,8aR)-6-(ethylthio)-2-phenylhexahydropyrano[3,2-d][1,3]dioxine-7,8-diyl dibenzoate: (13)

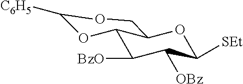

Benzoyl chloride (16.0 mmol, 4 eq.) and compound 12 (4.0 mmol, 1 eq.) were dissolved in dry pyridine (10 mL). The reaction was stirred at room temperature overnight. The reaction mixture was concentrated under vacuum followed by extraction with DCM/water. The organic layer was dried over Na₂SO₄ and concentrated under vacuum. Flash column chromatography was conducted (SiO₂: 5% to 25% EtOAc in hexanes) and the title compound 13 was isolated as a white foam in 91% yield (1892.80 mg, 3.64 mmol).

¹H NMR (500 MHz, CDCl₃) δ: 7.95-7.28 (m, 15H), 5.81 (t, J=9.4 Hz, 1H), 5.55 (s, 1H), 5.51 (t, J=9.6 Hz, 1H), 4.82 (d, J=9.9 Hz, 1H), 4.45 (dd, J=10.5, 4.8 Hz, 1H), 3.93 (t, J=9.5 Hz, 1H), 3.87 (t, J=10.3 Hz, 1H), 3.75 (td, J=9.6, 4.8 Hz, 1H), 2.77 (m, 2H), 1.26 (t, J=7.5 Hz, 3H).

¹³C NMR (126 MHz, CDCl₃) δ: 165.59, 165.32, 136.74, 133.30, 133.11, 129.88, 129.79, 129.39, 129.18, 129.05, 128.38, 128.30, 128.20, 126.12, 101.48, 84.51, 78.81, 73.21, 71.03, 71.00, 68.59, 24.43, 14.84.

HRMS for C₁₅H₂₀O₅S: [M+Na]⁺ calc.=543.1448; found=543.1446.

### (3aR,7aS)-2-(4-hydroxybutyl)-3a,4,7,7a-tetrahydro-1H-4, 7-methanoisoindole-1,3(2H)-dione: (14)

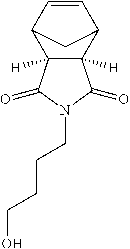 [SMILES: O=C1[C@@H]2C3C=CC(C3)[C@@H]2C(=O)N1CCCCO]

4-aminobutan-1-ol (33.5 mmol, 1.1 eq.) and cis-5-norbornene-exo-2,3-dicarboxylic anhydride (30.5 mmol, 1 eq.) were dissolved in dry toluene (20 mL). After adding TEA (1.5 mmol, 0.05 eq.), the reaction was stirred at 110° C. overnight. The reaction mixture was concentrated under vacuum. Flash column chromatography was conducted (SiO₂: 50% to 100% EtOAc in hexanes) and the title compound 14 was isolated as a white foam in 87% yield (6227.73 mg, 26.5 mmol).

¹H-NMR (500 MHz, CDCl₃): δ=6.28 (s, 2H), 3.65 (q, J=5.9 Hz, 2H), 3.54-3.45 (m, 2H), 3.26 (s, 2H), 2.67 (d, J=4.0 Hz, 2H), 2.05 (d, J=4.7 Hz, 1H), 1.68-1.60 (m, 2H), 1.60-1.53 (m, 2H), 1.51 (dd, J=10.1, 7.4 Hz, 1H), 1.21 (d, J=9.3 Hz, 1H).

¹³C-NMR (126 MHz, CDCl₃): δ=178.19, 137.81, 62.08, 47.80, 45.14, 42.73, 38.39, 29.81, 24.31.

HRMS for C₁₃H₁₇NO₃: [M+Na]⁺ calc.=258.1101; found=258.1111.

### (4aR,6R,7R,8S,8aR)-6-(4-((3aR,7aS)-1,3-dioxo-1,3,3a,4,7,7a-hexahydro-2H-4,7-methanoisoindol-2-yl)butoxy)-2-phenylhexahydropyrano[3,2-d][1,3]dioxine-7,8-diyl dibenzoate: (15)

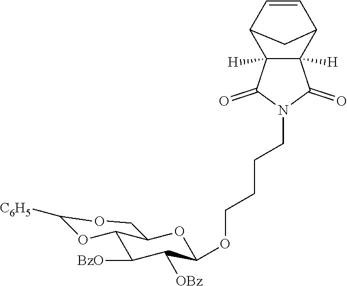

(13) (3.3 mmol, 1 eq.) and (14) (3.9 mmol, 1.2 eq.) were dissolved in dry DCM (10 mL) with 3 Å molecular sieves. After adding NIS (5.0 mmol, 1.5 eq.) followed by TMSOTf (0.2 mmol, 0.05 eq.), the reaction was stirred at −40° C. for 2 hours. The reaction mixture was quenched by adding TEA and was concentrated under vacuum. The crude product was extracted with water/DCM followed by washing with water. Flash column chromatography was conducted (SiO₂: 10% to 60% EtOAc in hexanes) and the title compound 15 was isolated as a white foam in 67% yield (1532.22 mg, 2.21 mmol).

¹H-NMR (500 MHz, CDCl₃): δ=8.02-7.92 (m, 4H), 7.56-7.39 (m, 5H), 7.39-7.26 (m, 6H), 6.26 (t, J=1.9 Hz, 2H), 5.80 (t, J=9.5 Hz, 1H), 5.57 (s, 1H), 5.48 (dd, J=9.4, 7.8 Hz, 1H), 4.82 (d, J=7.8 Hz, 1H), 4.45 (dd, J=10.6, 4.9 Hz, 1H), 4.02-3.84 (m, 3H), 3.72 (td, J=9.7, 4.9 Hz, 1H), 3.58 (dt, J=9.4, 5.8 Hz, 1H), 3.43-3.30 (m, 2H), 3.25-3.16 (m, 2H), 2.60 (s, 2H), 1.54 (m, 4H), 1.48-1.40 (d, J=9.6 Hz, 1H), 1.18-1.11 (d, J=9.6 Hz, 1H).

¹³C-NMR (126 MHz, CDCl₃): δ=177.91, 165.62, 165.14, 137.79, 136.85, 133.17, 133.10, 129.81, 129.78, 129.44, 129.35, 129.02, 128.38, 128.31, 128.20, 126.14, 101.65, 101.45, 78.85, 72.50, 72.15, 69.49, 68.67, 66.60, 47.74, 45.12, 42.71, 38.04, 26.85, 24.28.

HRMS for C₄₀H₃₉NO₁₀: [M+Na]⁺ calc.=716.2466; found=716.2465.

### (3aR,7aS)-2-(4-(((2R,3R,4S,5S,6R)-3,4,5-trihydroxy-6-(hydroxymethyl)tetrahydro-2H-pyran-2-yl)oxy)butyl)-3a,4,7,7a-tetrahydro-1H-4, 7-methanoisoindole-1,3(2H)-dione: (16)

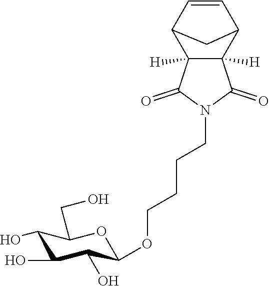

15 (0.4 mmol, 1 eq.) and DTT (0.8 mmol, 2 eq.) were dissolved in dry DCM (10 mL). After adding CSA (0.04 mmol, 0.1 eq.), the reaction was stirred at room temperature for 7 hours. The reaction mixture was quenched by adding TEA and was extracted with ethyl acetate/water. After drying the solvent under vacuum, the crude product was dissolved in dry MeOH (10 mL). After adding NaOME (0.04 mmol, 0.1 eq.), the reaction was stirred at room temperature for 2 hours. The reaction was neutralized with proton exchange resin. Flash column chromatography was conducted (SiO₂: 0% to 10% MeOH in DCM) and the title compound 16 was isolated as a white solid in 62% yield (98.46 mg, 0.25 mmol).

¹H-NMR (500 MHz, MeOD): δ=6.35 (t, J=1.9 Hz, 2H), 4.26 (d, J=7.8 Hz, 1H), 3.98-3.85 (m, 2H), 3.69 (dd, J=11.9, 5.0 Hz, 1H), 3.59 (d, J=9.8 Hz, 1H), 3.52 (t, J=6.9 Hz, 2H), 3.39-3.25 (m, 5H), 3.23-3.15 (m, 3H), 2.74 (d, J=1.4 Hz, 2H), 1.73-1.58 (m, 4H), 1.55-1.48 (m, 1H), 1.26 (d, J=9.7 Hz, 1H).

¹³C-NMR (126 MHz, MeOD): δ=178.79, 137.51, 102.95, 76.68, 76.52, 73.71, 70.25, 68.60, 61.39, 44.91, 42.12, 37.93, 26.74, 24.12.

HRMS for C₁₉H₂₇NO₈: [M+Na]⁺ calc.=420.1629; found=420.1638.

### General Polymerization Methods

Each polymer was made using these standard conditions unless otherwise stated. Monomer was placed under high vacuum overnight followed by procurement of freshly dried DMF via a VAC solvent purification system (SPS) in an oven dried, N₂ backfilled collection flask. The following steps were completed under a nitrogen atmosphere in a PLAS-LABS 810-series glove box. The monomer (or both monomers for Co-CPC generation) was dissolved in DMF followed by addition of the Grubbs M300® catalyst (Sigma: 682330) solution (in DMF) at the target monomer to catalyst ratio to a final catalyst concentration of 1.5 mM. The polymerization was removed from the glove box and sealed using PTFE tape and parafilm. The reaction was heated at 60° C. while shaking at −1200 rpm using a Benchmark Scientific Heating and Cooling Shaker (H5000-HC) for 2 to 4 hours depending on the target polymer length. Polymer termination was accomplished via the addition of ethyl vinyl ether.

### Kinetics

Kinetics measurements were run on at 60° C. in DMF-d₇. The general polymerization method was followed using a J-young air-free NMR tube with a time=0 point taken prior to catalyst addition and the first time point taken as quickly as the sample could be submitted. The NMR took a ¹H NMR spectrum every 120 or 180 seconds at least 13 times. Final catalyst concentration was 1.65 mmol for the APOH monomer however was 0.6 mM and 0.47 mM for the galactose and lactose glycomonomers respectively, due to the large quantity of monomer necessary to reach at least 1.5 mM of catalyst in the required minimum NMR volume.

Experiments with Full Length C-Terminus His-Tag Labeled Galectin-3

The C-terminus his-tag labeled galectin-3 was first diluted to 800 nm and NTA-Atto-488 fluorophore (Sigma Aldrich: 39625) diluted to 80 nm, both in 1×PBS. A 1:1 solution of galectin-3 and fluorophore was made and set aside while the polymers were diluted. Each polymer was dissolved in 1×PBS to 1 mM followed by a 1:1 serial dilution 16 times with 1×PBS. The highest concentration of the polymer was 20 μM or 50 μM depending on the polymer's anticipated strength of binding. The galectin-3: fluorophore mixture was added to each polymer solution to create a 1:1 mixture of polymer to fluorescently labeled protein with final fluorophore concentration at 20 nM. The mixture was allowed to incubate at room temperature for 1.5-2 hours shieled from light. Once equilibrated, the manufacturer's instructions were followed to load and run the thermophoresis experiment. The capillary was run at least three times.

The data was fit using the Nanotemper K_{d} model with data originating from 5 or 10 seconds post-laser ignition. The resulting K_{d} values were then averaged with standard deviation determined.

The copolymers made for the MST experiments utilized mono-Gal₁₀ as the galactose monomer and mono-OH₇ as the diluent. The copolymers used in the remainder of the manuscript experiments utilized mono-Gal₇ as the galactose monomer and mono-OH₇ as the diluent.

### Coarse-Grained Mapping of CPCs

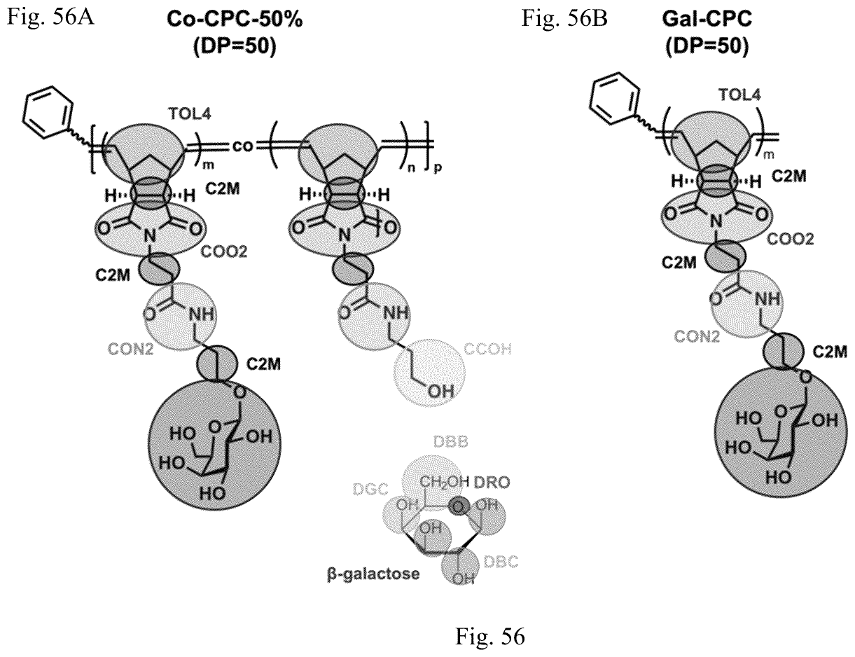

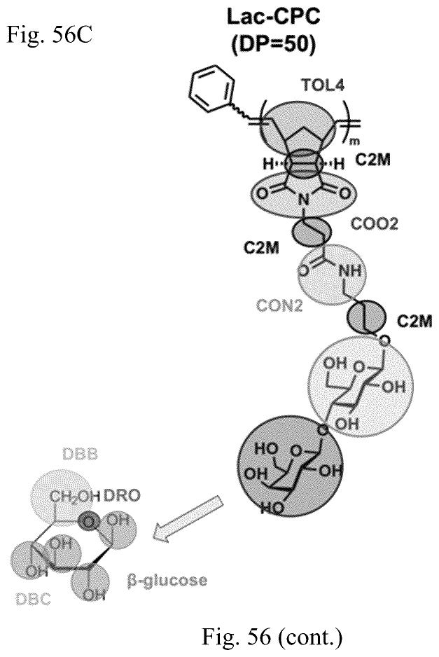

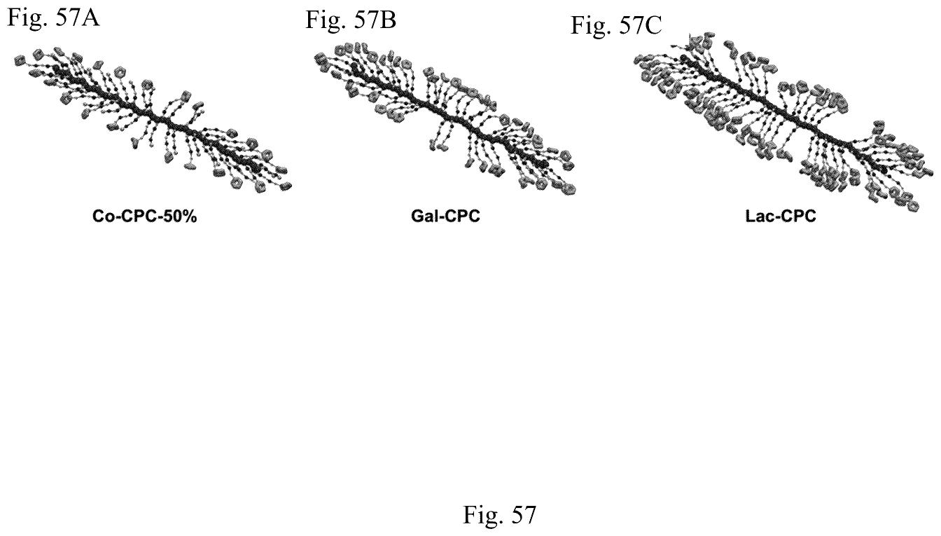

The three CPC structures, modeled as part of this study, are shown in FIG. 56. All structures contained some similarities including—a modified norbornene backbone, consistent side chain groups, and unique functional groups. The backbone and side chain groups of the CPCs were modeled using previously published TOL4, C2M, CCO2, and CON2 beads (An et al., 2019, J. Phys. Chem. B, 123, 909). For the carbohydrate functional groups, however, a novel model with a 2:1 mapping scheme was utilized, to accurately capture the intricate pyranose ring structures of these molecules. The CPC structures were generated using an in-house Python code and their equilibrium bond and angle values were ascertained by mapping CG beads onto energy-minimized atomistic configurations of the side chains obtained from AVOGADRO⁸. The angles between adjacent side chains of the CPCs were defined to reasonably capture Cis and Trans configurations between side chains. An angle of zero between adjacent side chains was defined as the Cis configuration whereas an angle of 180 degrees was defined as the Trans configuration. All systems were randomly generated with a Cis/Trans ratio of ~50% as shown in FIG. 57. The CPCs were solvated in a cubic box of side length 180 Å, with explicit 1-site water beads, each representing 2 water molecules, using the PACKMOL package. CHARMM-type FF equation was used to define bonded and nonbonded interactions between CG beads.

### Carbohydrate CG Model

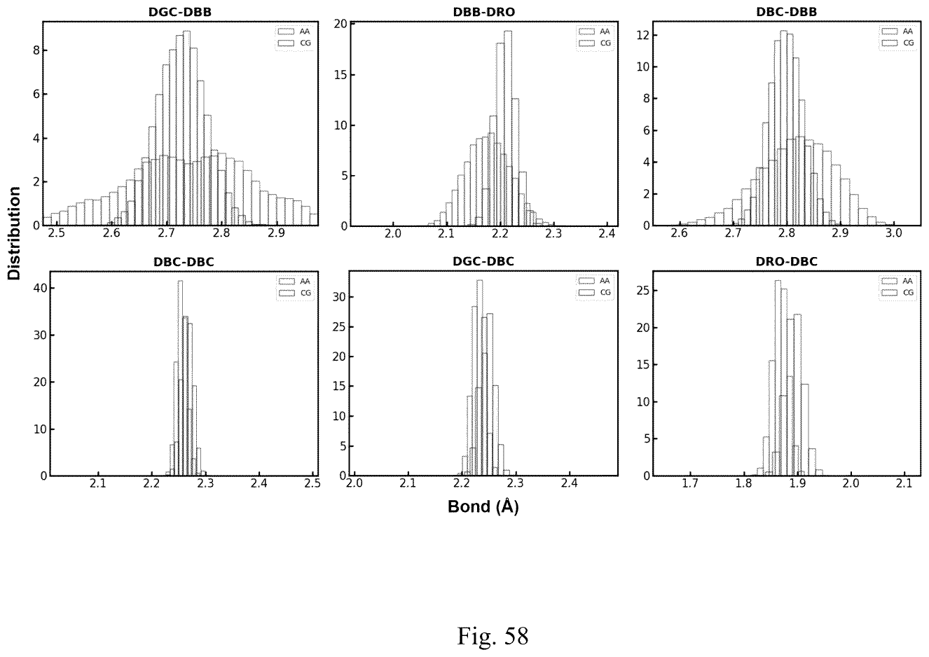

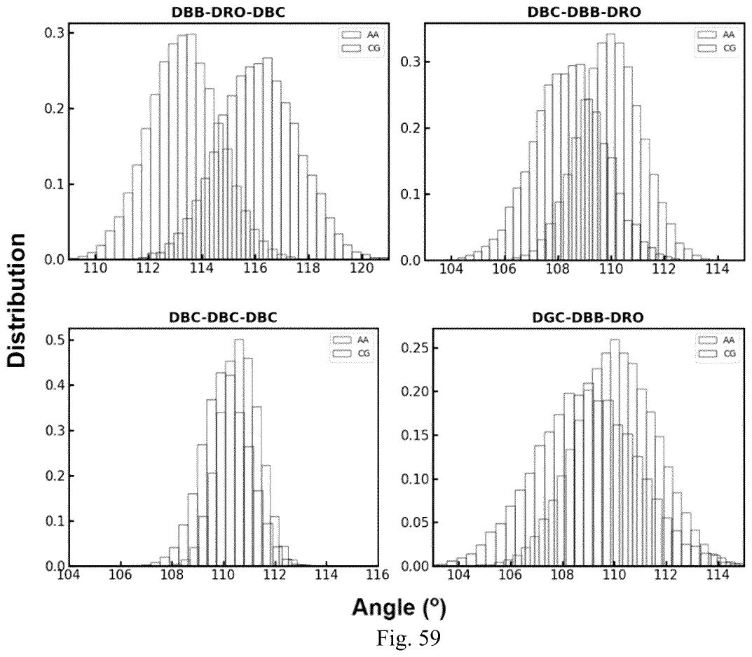

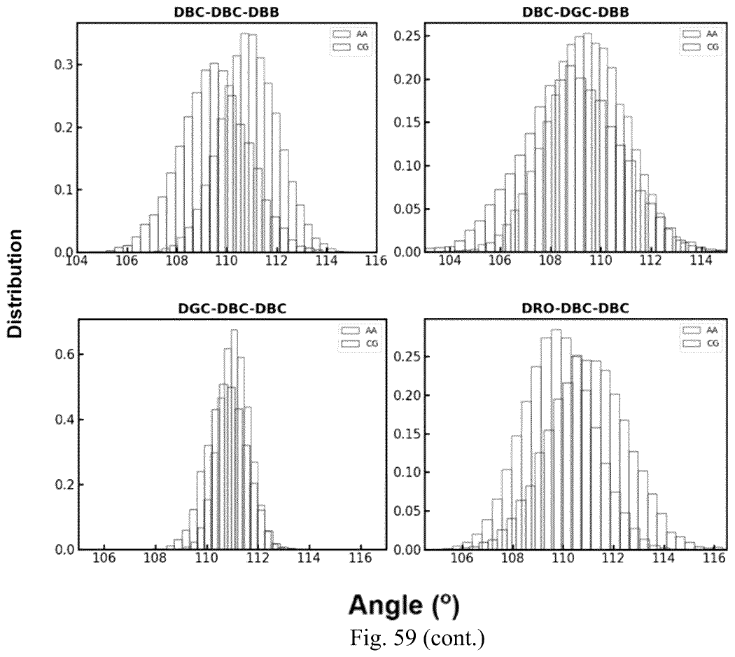

The transferable CG models for amino acids and hydrocarbons developed in previous studies were used to represent the side chains and backbone of CPCs, respectively. β-galactose and β-glucose were mapped according to FIG. 56 and tuned to show good agreement with structural (FIG. 58, FIG. 59) and bulk properties obtained from atomistic (AA) MD simulations using carbohydrate FFs, CHARMM (Guvench et al., 2008, J. Comp. Chem., 29, 2543; Vanommeslaeghe et al., 2010, J. Comp. Chem., 31, 671), GLYCAM06j (Kirschner et al., 2008, J. Comp. Chem., 29, 622), and OPLS-AA (Kony et al., 2002, J. Comp. Chem., 23, 1416).

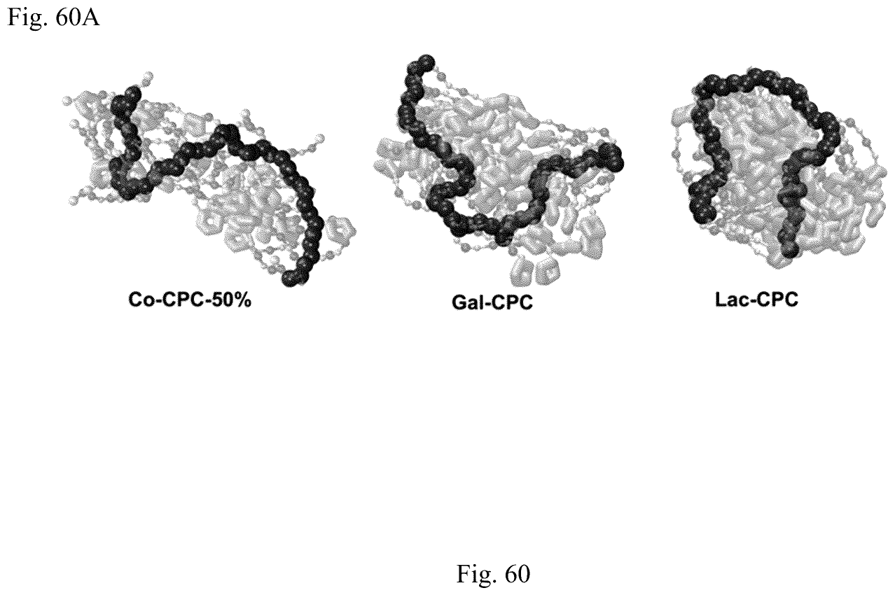

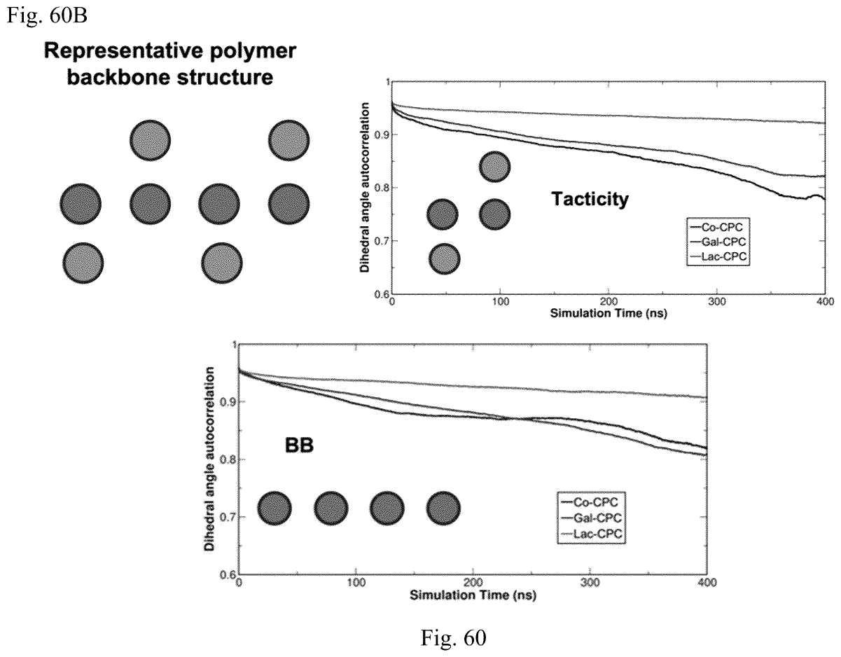

In order to determine whether the modeled CPC systems maintained their structural features throughout the simulations, the dihedral angle autocorrelation function was calculated for all systems (FIG. 60) and the lactose CPC showed lowest decay among the three systems (Hanwell et al., 2012, J. Cheminfor., 4, 17; Martinez et al., 2009, J. Comp. Chem., 30, 2157). Furthermore, all systems showed a high autocorrelation (>0.8) indicating that the backbone orientation and tacticity was maintained throughout the simulations.

To examine the extent of collapse in different CPCs, the Radius of Gyration (R_{g}) was calculated for all the systems. FIG. 7 shows the evolution of R_{g} and equilibrated R_{g} data, respectively, for all three CPCs, averaged over three different starting configurations. It was observed that the extent of the collapse was slightly lower for the lactose homopolymer (Lac-CPC) compared to copolymer (Co-CPC-50%) and galactose homopolymer (Gal-CPC). This can be attributed to the steric effects of the larger disaccharide functional groups attached to the Lac-CPC system. Furthermore, it was found that while the R_{g} distribution for the entire CPCs in the Co-CPC, and Gal-CPC systems was similar, the backbone R_{g} for the galactose homopolymer system was lower compared to the other systems pointing towards a more collapsed backbone.

### General Procedure for Dynamic Light Scattering (DLS)

Each polymer was dissolved in 18.2 MQ water (milliQ water) or 1×PBS and placed on a rocker for at least 8 hours. The sample was then transferred to a disposable plastic cuvette and was analyzed using a Zetasizer Nano ZS (Dispersant: H₂O RI=1.330, Viscosity=0.8872 or 1×PBS RI=1.332, Viscosity=0.9043; Material: RI=1.45, Absorption=0.001; T=25° C.) with three runs per measurement, 10 seconds per run, and at least 3 measurements averaged together.

### Galectin-3 Western Blot

Cells (4T1 or LNCaP) were washed with PBS and lysed using RIPA buffer with Halt phosphatase and protease inhibitor cocktail (Thermo Fisher: 78440). Protein concentration of the cell lysate was determined using the Pierce BCA assay (Thermo Fisher: 23225, 30 minute, 37° C. protocol). 15 g of protein was incubated at 60° C. for 15 minutes with Laemmli loading dye and 100 mM DTT. Samples were separated in 4-20% SDS-PAGE gel and transferred to a low fluorescence PVDF membrane. The membrane was blocked with Licor Intercept blocking buffer then washed with PBST. The membrane was incubated with a 1:500 dilution of anti-galectin-3 (Thermo Fisher: 14-5301-82) and 1:2000 dilution of anti-beta tubulin (Thermo Fisher: MA516308) in Licor Intercept blocking buffer at 4° C. overnight. Following incubation, the membrane was washed with PBST then was incubated with 1:1000 dilution of goat anti-rat 647 (Thermo Fisher: A21247) and 1:1,000 of goat-anti mouse 488 (Thermo Fisher: A28175) in Licor Intercept blocking buffer for 120 minutes at room temperature in the dark. The membrane was washed with PBST followed by PBS then imaged with a ChemiDoc Imaging Station.

### Cytotoxicity

Cells were seeded in a 96-well plate at 2,500 cells per well for both 4T1 and LNCaP lines. Following growth overnight, cells were treated with PBS, galactose (1 mM), glucose control polymer (10 μM), or experimental treatment polymers at final concentrations of 10 μM. At 4 hours post treatment, CCK-8 assay (Fisher Scientific: NC9864731) was performed per the manufacturer's instructions. The plate was analyzed using a BioTek Synergy H1 microplate reader.

### Cellular Viability Proliferation

Cells were seeded in a 96-well plate at 2,500 cells per well for both 4T1 and LNCaP lines. Following growth overnight, cells were treated with PBS, galactose (1 mM), glucose control polymer (10 μM), or experimental treatment polymers at final concentrations of 10 μM. At 24 hours post treatment, CCK-8 assay (Fisher Scientific: NC9864731) was performed per the manufacturer's instructions. The plate was analyzed using a BioTek Synergy H1 microplate reader.

### Live Cell Internalization

Cells were seeded in Nunc™ Lab-Tek™ II Chambered Coverglass (Thermo Fisher: 155409) at 50,000 cells per well. Following growth overnight, cells were treated with Cy5 labeled Gal-CPC, DP=100 polymer at 5 μM for 4 hours. Cells were washed twice with PBS and then stained with CellBrite™ Steady 488 Membrane live cell stain (Biotium: 30106-T) via the media exchange protocol for 30 minutes. Cells were then washed twice with media and imaged using a Nikon W1 spinning disk confocal microscope. Images were taken with a 20× (Plan Apochromat λ 20×; NA—0.75; WD—1.0 mm) or 100× (Plan Apochromat λ 100× (Oil); NA—1.45; WD—0.13 mm) objective.

Fixed Cell Internalization with Galectin-3 Antibody Staining

Cells were seeded in Nunc™ Lab-Tek™ II Chambered Coverglass (Thermo Fisher: 155409) at 50,000 cells per well. Following growth overnight, cells were treated with Cy5 labeled Gal-CPC DP=100 polymer at 5 μM for 4 hours. Cells were washed 4× with PBS and then incubated in 4% PFA for 20 minutes. Cells were permeabilized 3× with PBS followed by incubation for 10 minutes in a 0.1% w/v saponin solution in PBS. Cells were rinsed 3× with PBS for 5 minutes each. Cells were then blocked (PBS with 0.1% saponin and 10% normal goat serum (NGS)) for 1 hour. The cells were incubated in a 1:100 dilution of anti-galectin-3 (Thermo Fisher: 14-5301-82) in PBS with 1% NGS and 0.1% saponin at 4° C. overnight. The cells were rinsed 4× in PBS for 5 minutes each followed by incubation with a 1:1000 dilution of goat anti-rat Alexa fluor-488 (Thermo Fisher: A11006) in PBS with 1% NGS and 0.1% saponin for 1 hour in the dark. The cells were washed 3× with PBS for 5 minutes each followed by counterstaining with DAPI following the manufacturers recommendations (Thermo Fisher: R37606) for 5 minutes. The cells were then washed 3× with PBS and kept hydrated until imaging. Images were taken using a Nikon WI spinning disk confocal microscope with a 20× (Plan Apochromat λ 20×; NA—0.75; WD—1.0 mm) or 100× (Plan Apochromat λ 100× (Oil); NA—1.45; WD—0.13 mm) objective.

### Scratch Assay

Cells were seeded in a 24-well plate and allowed to grow until confluent. Following confluence, a p200 micropipette tip was used to make scratches in each well. The media was changed to remove upended cells and the wells were imaged using a Nikon SMZ800N Stereoscope fitted with a Nikon DS-Ri2 color camera. Wells were then treated with PBS, galactose (1 mM) or experimental treatment polymers at final concentrations of 1, 2.5, 5, or 10 μM. Following treatment for 24 hours, cells were imaged then fixed in 4% PF A and stained with crystal violet. Cells were imaged again.

Analysis of the scratches was completed using ImageJ. the width of each scratch was determined by drawing 10 individual horizontal lines across the scratch, making sure to measure the width along the entirety of the scratched area. All measurements were then averaged together and compared to the average of 10 individual horizontal lines across each scratch at 24 h.

Ultimately, the complex, multifunctional role of carbohydrates in biology relies not solely on their molecular composition, but also on their broader macro- and supramolecular architecture. As such, the emergence of novel methodologies aimed at interrogating and understanding the functions of glycans in biological contexts presents exciting prospects for delving deeper into the intricacies of the natural world. In this vein, CPCs have been developed herein as tools for dissecting the impact of carbohydrate multivalency on β-galactoside recognition by Gal-3, a pivotal player in both health and disease. Present investigations have found that maximum grafting density is required for optimal recognition of the present materials by this lectin, highlighting the dominance of multivalency over individual ligand specificity in fostering high avidity interactions. Furthermore, computational analyses reveal that fully grafted CPCs boast the greatest solvent accessible surface area due to reduced carbohydrate-carbohydrate interactions, offering valuable insights into the spatial organization of glycans in the glycocalyx and their mode of interaction with Gal-3. This knowledge translates into substantial advancements in the biological function of these materials, as evidenced by the ability of Gal-CPCs to effectively suppress both the proliferation and migration of 4T1 triple negative breast cancer cells. Thus, these materials not only advance fundamental comprehension of glycans in biology but also offer a promising route for cancer therapeutics from readily available, simple β-galactosides.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A carbohydrate-polymer conjugate comprising a structure represented by Formula (I): 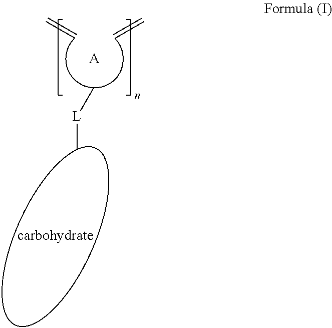
wherein:
A represents a repeat unit selected from the group consisting of C₅-C₇ alkyl, C₅-C₇ heteroalkyl, C₅-C₇ aryl, C₅-C₇ heteroaryl, or C₅-C₇ cycloalkyl;
n represents an integer from 10-500;
L represents a divalent organic linker; and
wherein L forms a direct bond to an anomeric position of the carbohydrate.

2. The carbohydrate-polymer conjugate of claim 1, wherein ring A is represented by one of the following structures: 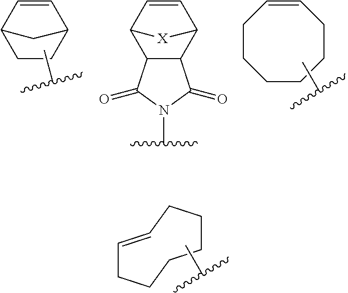
wherein the wave line represents a bond to L;
X represents O, S, NR¹, or CR²R³;
R¹, R², and R³ each independently represent a substituent selected from the group consisting of hydrogen, halide, hydroxy, C₁-C₃₀ alkyl, C₁-C₃₀ alkyl halide, C₁-C₃₀ alkoxy, and any combination thereof, and
each structure is independently optionally further substituted.

3. The carbohydrate-polymer conjugate of claim 1, wherein the polymer backbone is unsaturated.

4. The carbohydrate-polymer conjugate of claim 1, wherein L is represented by one of the following structures: 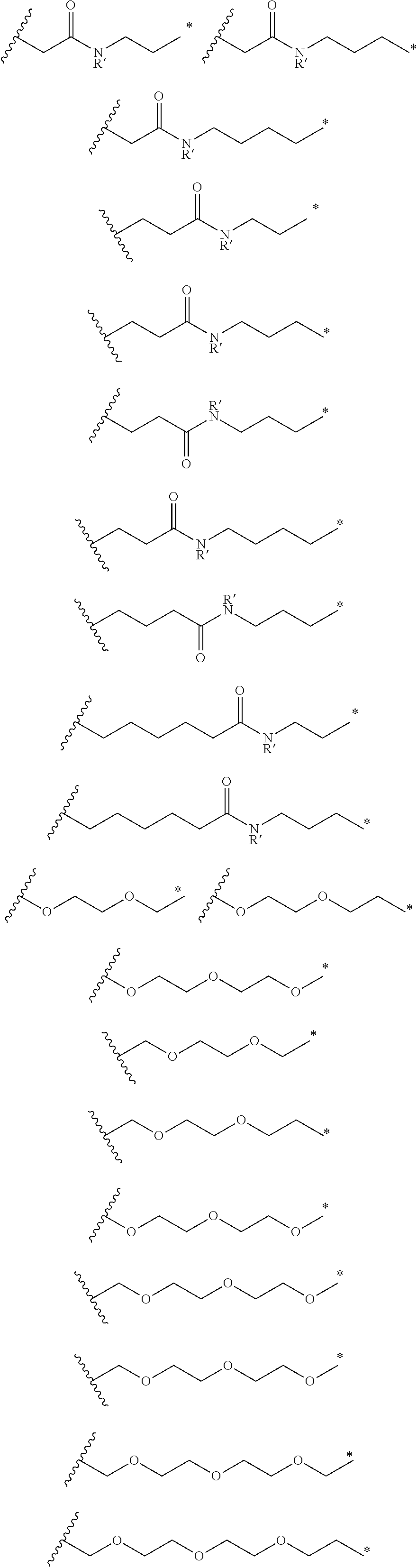
wherein the wavy line represents a bond to ring A;
* represents a bond to the carbohydrate; and
R′ represents a substituent selected from the group consisting of hydrogen, halide, hydroxy, C₁-C₃₀ alkyl, C₁-C₃₀ alkyl halide, C₁-C₃₀ alkoxy, and any combination thereof.

5. The carbohydrate-polymer conjugate of claim 1, wherein L comprises a C₁-C₃₀ alkyl which is optionally further substituted.

6. The carbohydrate-polymer conjugate of claim 1, wherein the carbohydrate is selected from the group consisting of a monosaccharide, a disaccharide, an oligosaccharide, a polysaccharide, any stereochemical isomer thereof, and any combination thereof.

7. The carbohydrate-polymer conjugate of claim 1, wherein the carbohydrate is selected from the group consisting of galactose, glucose, mannose, allose, altrose, gulose, idose, talose, psicose, fructose, sorbose, tagatose, and any stereochemical isomer thereof.

8. The carbohydrate-polymer conjugate of claim 1, wherein the carbohydrate-polymer conjugate is a copolymer comprising a repeat unit of at least two monomers, wherein at least one monomer comprises galactose.

9. The carbohydrate-polymer conjugate of claim 1, wherein the carbohydrate is acetylated.

10. A composition comprising the carbohydrate-polymer conjugate of claim 1.

11. A method of synthesizing a carbohydrate-polymer conjugate using ring-opening metathesis polymerization (ROMP), comprising the steps of:
providing a monomer;
polymerizing the monomer using a ROMP catalyst; and
isolating the carbohydrate-polymer conjugate;
wherein the monomer is represented by Formula (I): 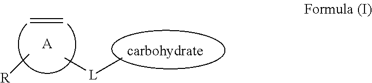
wherein:
R represents a substituent on ring A selected from the group consisting of hydrogen, halide, hydroxy, C₁-C₃₀ alkyl, C₁-C₃₀ alkyl halide, C₁-C₃₀ alkoxy, and any combination thereof, and
L represents a divalent organic linker.

12. The method of claim 11, wherein the degree of polymerization is about 10 to about 500.

13. The method of claim 11, wherein the polymerization is performed at about 60° C.

14. A method comprising the step of administering to the subject a composition comprising a carbohydrate-polymer conjugate comprising a structure represented by Formula (I): 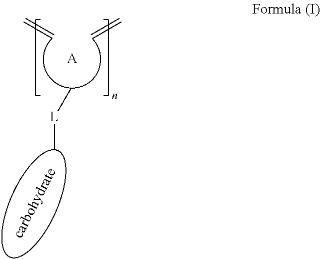
wherein:
A represents a repeat unit selected from the group consisting of C₅-C₇ alkyl, C₅-C₇ heteroalkyl, C₅-C₇ aryl, C₅-C₇ heteroaryl, or C₅-C₇ cycloalkyl;
n represents an integer from 10-500;
L represents a divalent organic linker; and
wherein L forms a direct bond to an anomeric position of the carbohydrate.

15. The method of claim 14, wherein the subject has cancer.

16. The method of claim 15, wherein the composition suppresses proliferation of a cancer cell.

17. The method of claim 15, wherein the composition inhibits tumor growth.

18. The method of claim 15, wherein the composition inhibits at least one cancer cell from metastasizing.

19. The method of claim 14, wherein the composition binds to a lectin.

20. The method of claim 14, wherein the composition binds to a target protein selected from Gal-3, MGL, ASGPR, MBL, DC-SIGN, Langerin, GLUT-1, GLUT-2, GLUT-3, variants thereof, and combinations thereof.
